SEQUENCE LISTING (21 sequences)

SEQ ID NO: 1 - protein, 214 aa, SARS-CoV-2
  REGION 1..214: note = misc_feature - SARS-CoV-2(Delta)RBD; GenBank ON196573.1
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTN

SEQ ID NO: 2 - protein, 222 aa, SARS-CoV
  REGION 1..222: note = misc_feature - SARS-CoV RBD; GenBankYP_009825051.1
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLT

SEQ ID NO: 3 - protein, 212 aa, MERS-CoV
  REGION 1..212: note = misc_feature - MERS-CoV RBD; GenBankAFS88936.1
QAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

SEQ ID NO: 4 - protein, 20 aa, synthetic construct
  REGION 1..20: note = Synthetic: Linker L20
GGGGSGGGGSGGGGSGGGGS

SEQ ID NO: 5 - protein, 688 aa, synthetic construct
  REGION 1..688: note = Synthetic: Full construct AG1 S2S1ML20 - SARS-CoV-2 (Delta) RBD-linker-SARS-CoV RBD-linker-MERS-CoV RBD (FIG. 12) S2S1ML20
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNGGGGSGGGGSGGGGSGGGGSNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTGGGGSGGGGSGGGGSGGGGSQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

SEQ ID NO: 6 - protein, 668 aa, synthetic construct
  REGION 1..668: note = Synthetic: Full construct S2S1ML10
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNGGGGSGGGGSNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTGGGGSGGGGSQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

SEQ ID NO: 7 - protein, 678 aa, synthetic construct
  REGION 1..678: note = Synthetic: Full construct S2S1ML15
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNGGGGSGGGGSGGGGSNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTGGGGSGGGGSGGGGSQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

SEQ ID NO: 8 - protein, 441 aa, synthetic construct
  REGION 1..441: note = Synthetic: Full construct S2ML15
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNGGGGSGGGGSGGGGSQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

SEQ ID NO: 9 - protein, 451 aa, synthetic construct
  REGION 1..451: note = Synthetic: Full construct S2SL15
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNGGGGSGGGGSGGGGSNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLT

SEQ ID NO: 10 - protein, 15 aa, synthetic construct
  REGION 1..15: note = Synthetic: Linker L15
GGGGSGGGGSGGGGS

SEQ ID NO: 11 - protein, 10 aa, synthetic construct
  REGION 1..10: note = Synthetic: Linker L10
GGGGSGGGGS

SEQ ID NO: 12 - protein, 214 aa, SARS-CoV-2
  REGION 1..214: note = misc_feature - SARS-CoV-2(WA1)RBD; GenBank UJH58758.1
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 13 - protein, 214 aa, SARS-CoV-2
  REGION 1..214: note = misc_feature - SARS-CoV-2(BA.2)RBD; GenBank UNE80990.1
RVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTN

SEQ ID NO: 14 - protein, 212 aa, Pangolin coronavirus
  REGION 1..212: note = misc_feature - Pangolin coronavirus RBD; GenBank MT040334.1
RVQPTISIVRFPNITNLCPFGEVFNASKFASVYAWNRKRISNCVADYSVLYNSTSFSTFKCYGVSPTKLNDLCFTNVYADSFVVKGDEVRQIAPGQTGVIADYNYKLPDDFTGCVIAWNSVKQDALTGGNYLYRLFRKSKLKPFERDISTEIYQAGSTPCNGQVGLNCYYPLERYGFHPTTGVNYQPFRVVVLSFELLNGPATVCGPKLSTT

SEQ ID NO: 15 - protein, 213 aa, SARS-CoV
  REGION 1..213: note = misc_feature - SARS-CoV RBD; GenBank YP_009825051.1
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTD

SEQ ID NO: 16 - protein, 213 aa, Bat SARS-like coronavirus
  REGION 1..213: note = misc_feature - WIV1 RBD; GenBank AGZ48828.1
RVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTD

SEQ ID NO: 17 - protein, 213 aa, Bat SARS-like coronavirus; identical to SEQ ID NO: 16
  REGION 1..213: note = misc_feature - Rs3367 RBD; GenBank AGZ48818.1

SEQ ID NO: 18 - protein, 213 aa, SARS coronavirus
  REGION 1..213: note = misc_feature - SHC014 RBD; GenBank QJE50589.1
RVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLSTD

SEQ ID NO: 19 - protein, 212 aa, MERS-CoV; identical to SEQ ID NO: 3
  REGION 1..212: note = misc_feature - MERS-CoV Clade A; GenBank AFS88936.1

SEQ ID NO: 20 - protein, 212 aa, MERS-CoV; identical to SEQ ID NO: 3
  REGION 1..212: note = misc_feature - MERS-CoV Clade B; GenBank AKJ80137.2

SEQ ID NO: 21 - protein, 212 aa, MERS-CoV
  REGION 1..212: note = misc_feature - MERS-CoV Clade C; GenBank AVN89387.1
QAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPFKYSYINKCSRLLSDDRTEVPQLVNANQYSPCVSIVPSTVWEDGDYYRKQLSPLEGGGWLVASGSTVAMTEQLQMGFGITVQYGTDTNSVCPKF

(19) United States
(12) Patent Application Publication
(10) Pub. No.: US 2025/0108106 A1
(43) Pub. Date: Apr. 3, 2025

(54) NANOENCAPSULATED PHARMACEUTICAL COMPOSITION AND USE THEREOF

(71) Applicants: Fulgent Genetics, Inc., El Monte, CA (US); Fudan University, Shanghai (CN)

(72) Inventors: Lu Lu, Newark, DE (US); Ray Yin, Wilmington, DE (US); Shibo Jiang, Shanghai (CN); Zezhong Liu, Shangai (CN); Ming Hsieh, Pasadena, CA (US); Jie Zhou, Shanghai (CN); Xinling Wang, Shanghai (CN); Qian Wang, Shanghai (CN); Wei XU, Shanghai (CN); Jing Pan, Newark, DE (US); Yubei Zhang, Hockessin, DE (US); Kai Qi, Wilmington, DE (US); Qun Sun, Wilmington, DE (US); Lin Wang, Hockessin, DE (US); Zhiying Zou, Newark, DE (US); Chunlin Tao, Carlsbad, CA (US)

(21) Appl. No.: 18/829,855

(22) Filed: Sep. 10, 2024

Related U.S. Application Data
(63) Continuation of application No. PCT/US23/64235, filed on Mar. 13, 2023.
(60) Provisional application No. 63/319,585, filed on Mar. 14, 2022.
(60) Provisional application No. 63/335,408, filed on Apr. 27, 2022.
(60) Provisional application No. 63/382,149, filed on Nov. 3, 2022.

(51) Int. Cl. A61K 39/215; A61K 9/51; A61K 31/496; A61K 31/5377; A61K 39/00; A61K 39/39; A61K 45/06; A61P 31/14; C12N 7/00

(52) U.S. Cl. CPC A61K 39/215; A61K 9/5146; A61K 31/496; A61K 31/5377; A61K 39/39; A61K 45/06; A61P 31/14; C12N 7/00; A61K 2039/55555; A61K 2039/575

(57) ABSTRACT

This disclosure is directed to a pharmaceutical composition for treating or preventing a disease. The pharmaceutical composition can comprise a polymer-drug nanoaggregate having a polymer and at least one bioactive agent that can comprise STING polypeptide, a nucleic acid encoding said STING polypeptide, a STING inhibitor, a STING activator, a STING agonist, a STING antagonist, a STING modulating molecule, or a combination thereof. The pharmaceutical composition can be a vaccine or an adjuvant for a vaccine. This disclosure is also directed to a method for treating or preventing a disease using the pharmaceutical composition. The disease can include infectious diseases caused by viruses or other pathogens, for example, influenza, rabies, or respiratory illnesses such as severe acute respiratory syndrome (SARS) caused by coronaviruses, such as MERS-CoV, SARS-CoV, and Coronavirus Disease 2019 (COVID-19) caused by the virus SARS-CoV-2 and its variants.

### RELATED APPLICATIONS

The application is a continuation of International Application No. PCT/US2023/64235, filed Mar. 13, 2013, which claims the benefit of U.S. Provisional Application No. 63/319,585, filed Mar. 14, 2022, U.S. Provisional Application No. 63/335,408, filed Apr. 27, 2022, and U.S. Provisional Application No. 63/382,149, filed Nov. 3, 2022. All of which are hereby incorporated by reference in their entireties.

### REFERENCE TO ELECTRONIC SEQUENCE LISTING

The instant application contains a Sequence Listing in the specification below and an electronic version thereof is being submitted electronically in .XML format concurrently herewith and is herein incorporated by reference in entirety. Said .XML copy, created 4 Feb. 2023, is named, “010816-16093001.xml,” and is 33,108 bytes in size. The sequence listing contained in the concurrently filed .XML file is part of the specification and hereby is incorporated by reference in entirety.

### FIELD OF THE INVENTION

The present disclosure relates to a pharmaceutical composition and a method for treatment or prevention of a disease in patients in need thereof. The pharmaceutical composition can be a vaccine or an adjuvant.

### BACKGROUND

Synthetic polymers have been shown to have important applications in pharmaceutical formulations as an effective delivery vehicle or as an excipient.

Symmetrically branched polymers (SBP), such as, dendritic polymers including Starburst dendrimers (or Dense Star polymers) and Combburst dendrigrafts (or hyper comb-branched polymers), are some examples. Those polymers often possess: (a) a well-defined core, (b) at least two concentric dendritic layers (generations) with symmetrical (equal length) branches and branch junctures and (c) exterior surface groups, such as, polyamidoamine (PAMAM)-based branched polymers and dendrimers described in U.S. Pat. Nos. 4,435,548; 4,507,466; 4,568,737; 4,587,329; 5,338,532; 5,527,524; and 5,714,166. Other examples include polyethyleneimine (PEI) dendrimers, such as, those disclosed in U.S. Pat. No. 4,631,337; polypropyleneimine (PPI) dendrimers, such as, those disclosed in U.S. Pat. Nos. 5,530,092; 5,610,268; and 5,698,662; Frechet-type polyether and polyester dendrimers, core shell tectodendrimers and others, as described, for example, in, “Dendritic Molecules,” edited by Newkome et al., VCH Weinheim, 1996, “Dendrimers and Other Dendritic Polymers,” edited by Frechet & Tomalia, John Wiley & Sons, Ltd., 2001; and U.S. Pat. No. 7,754,500.

Combburst dendrigrafts are constructed with a core molecule and concentric layers with symmetrical branches through a stepwise synthetic method. In contrast to dendrimers, Combburst dendrigrafts or polymers are generated with monodisperse linear polymeric building blocks (U.S. Pat. Nos. 5,773,527; 5,631,329 and 5,919,442). Moreover, the branch pattern is different from that of dendrimers. For example, Combburst dendrigrafts form branch junctures along the polymeric backbones (chain branches), while Starburst dendrimers often branch at the termini (terminal branches). Due to the living polymerization techniques used, the molecular weight distributions (M_{w}/Mₙ) of those polymers (core and branches) often are narrow. Thus, Combburst dendrigrafts produced through a graft-on-graft process are well defined with M_{w}/Mₙ ratios often approaching 1.

SBPs, such as, dendrimers, are produced predominantly by repetitive protecting and deprotecting procedures through either a divergent or a convergent synthetic approach. Since dendrimers utilize small molecules as building blocks for the cores and the branches, the molecular weight distribution of the dendrimers often is defined. In the case of lower generations, a single molecular weight dendrimer often is obtained. While dendrimers often utilize small molecule monomers as building blocks, dendrigrafts use linear polymers as building blocks.

In addition to dendrimers and dendrigrafts, other SBPs can include symmetrical star-shaped or comb-shaped polymers, such as, symmetrical star-shaped or comb-shaped polyethyleneoxide (PEO), polyethyleneglycol (PEG), polyethyleneimine (PEI), polypropyleneimine (PPI), polyoxazoline (POX), polymethyloxazoline (PMOX), polyethyloxazoline (PEOX), polypropyloxazoline (PPOX), polymethylmethacrylate (PMMA), polystyrene, or polydimethylsiloxane.

Asymmetrically branched polymers (ABP) can have two different types: regular ABP and random ABP. Asymmetrically branched dendrimers or regular ABPs (reg-ABPs), often possess a core, controlled and well-defined asymmetrical (unequal length) branches and asymmetrical branch junctures as described in U.S. Pat. Nos. 4,289,872; 4,360,646; and 4,410,688. On the other hand, a random ABP (ran-ABP) possesses: a) no core, b) functional groups both at the exterior and in the interior, c) random/variable branch lengths and patterns (i.e., termini and chain branches), and d) unevenly distributed interior void spaces.

The synthesis and mechanisms of ran-ABPs, such as, those made from PEI, were reported by Jones et al., J. Org. Chem. 9, 125 (1944), Jones et al., J. Org. Chem. 30, 1994 (1965) and Dick et al., J. Macromol. Sci. Chem., A4 (6), 1301-1314, (1970)). Ran-ABP, such as those made of POX, poly(2-oxazoline), poly(2-methyloxazoline) (PMOX) and poly(2-ethyloxazoline) (PEOX), was reported by Litt (J. Macromol. Sci. Chem. A9 (5), 703-727 (1975)) and Warakomski (J. Polym. Sci. Polym. Chem. 28, 3551 (1990)). The synthesis of ran-ABPs often can involve a one-pot divergent or a one-pot convergent method.

A polymer can also be a homopolymer or a copolymer. A copolymer is a polymer, or a polymer backbone, polymerized from different monomers or different monomer repeating units. A homopolymer can relate to a polymer or a polymer backbone composed of the same repeat unit, that is, the homopolymer is generated from the same monomer. The monomer can be a simple compound or a complex or an assemblage of compounds where the assemblage or complex is the repeat unit in the homopolymer.

Although branched polymers, including SBPs and ABPs, have been used for drug delivery, those attempts are focused primarily on the chemical attachment of a drug to a polymer, or physical encapsulation of such drugs in the interior through unimolecular encapsulation (such as those described in U.S. Pat. Nos. 5,773,527; 5,631,329; 5,919,442; and 6,716,450). For example, dendrimers and dendrigrafts are believed to physically entrap bioactive molecules using unimolecular encapsulation approaches, as described in U.S. Pat. Nos. 5,338,532; 5,527,524; and 5,714,166 for dense star polymers, and U.S. Pat. No. 5,919,442 for hyper comb-branched polymers. Similarly, the unimolecular encapsulation of various drugs using SBPs to form a “dendrimer box” was reported in Tomalia et al., Angew. Chem. Int. Ed. Engl., 1990, 29, 138, and in “Dendrimers and Other Dendritic Polymers”, edited by Frechet & Tomalia, John Wiley & Sons, Ltd., 2001, pp. 387-424.

Branched core shell polymers with a hydrophobic core and a hydrophilic shell may be used to entrap a poorly water soluble drug through molecular encapsulation. Randomly branched and hyperbranched core shell structures with a hydrophilic core and a hydrophobic shell have also been used to carry a drug through unimolecular encapsulation and pre-formed nanomicelles (U.S. Pat. No. 6,716,450 and Liu et al., Biomaterials 2010, 10, 1334-1341). However, those unimolecular and pre-formed micelle structures are generated in the absence of a drug.

Block copolymers, such as, miktoarm polymers (i.e., Y shaped/AB₂-type star polymers) and linear (A)-dendritic (B) block copolymers, were observed to form stereocomplexes with paclitaxel (Nederberg et al., Biomacromolecules 2009, 10, 1460-1468 and Luo et al., Bioconjugate Chem. 2010, 21, 1216). Those block copolymers closely resemble traditional lipid or AB-type linear block copolymers, which are well known surfactants used for the generation of micelles. However, such branched block copolymers are difficult to make and thus, are not suitable for mass production.

Water insoluble or poorly water soluble bioactive agents are difficult to formulate. Typically, multiple surfactants, detergents and other materials or a complex high energy emulsification process can be needed. Large biological molecules, such as albumin, have been used in certain formulations for water insoluble paclitaxel, such as Abraxane® available from Celgene and Bristol-Myers Squibb under respective trademark. However, availability and large-scale production of such biological molecules have presented significant challenges.

Vaccines can help the body recognize and destroy certain targets, such as, microorganisms or other pathogens that cause infection. Adjuvants are typically used to modify, augment, or increase the efficacy or potency of a vaccine to provide better immunity to a particular disease. Aluminum-containing adjuvants have been used in vaccines since 1930s. Small amounts of aluminum are added to help the body build stronger immunity against microorganisms. Monophosphoryl lipid A (MPL) (also known as “AS04”) was used in U.S. vaccine (Cervarix®) and can have immune-boosting effects. An oil-in-water emulsion-based adjuvant, MF59, contains squalene, a naturally occurring oil found in many plant and animal cells, as well as in humans. The MF59 adjuvant has been used in Fluad® (an influenza vaccine licensed for adults aged 65 or older) in Europe since 1997 and in the United States since 2016. Another adjuvant, AS01B, is an adjuvant suspension used with the antigen component of the Shingrix vaccine. AS01B is made of monophosphoryl lipid A (MPL) and QS-21, a natural compound extracted from the Chilean soapbark tree (Quillaja saponaria Molina). AS01B is also a component of vaccines currently being tested in clinical trials, including malaria and HIV vaccines.

There is a continued need for new pharmaceutical formulations that can deliver drugs more effectively or to improve immunogenicity, such as, vaccine efficacy, by stimulating better immunity.

### SUMMARY

In some cases, the present invention is directed to a pharmaceutical composition comprising: a nanoaggregate comprising a polymer and at least one bioactive agent comprising at least one stimulator of interferon genes (STING) polypeptide or a part thereof, a nucleic acid encoding the STING polypeptide or a part thereof, a STING inhibitor, a STING activator, a STING agonist, a STING antagonist, a STING modulating molecule, or a combination thereof; and optionally a pharmaceutical suitable carrier; wherein the pharmaceutical composition is soluble in an aqueous solution to produce at least 1 mg/ml of a bioactive agent in the aqueous solution; wherein the polymer is water soluble; and wherein the polymer comprises: a first polymer comprising at least one first terminal group modified with H or a hydrophobic moiety and a second terminal group modified with a hydrophilic moiety, wherein the first terminal group comprises in a range of from 1% to 100% of H and 0% to 99% of the hydrophobic moiety that comprises a saturated or an unsaturated aliphatic hydrocarbon having 1 to about 22 carbons, an aromatic hydrocarbon, or a combination thereof; and the second terminal group comprises a group modified by an amine, amide, imine, imide, carboxyl, hydroxyl, ester, ether, acetate, phosphate, ketone, aldehyde, sulfonate, or a combination thereof; a second polymer; or a combination thereof.

In some cases, the pharmaceutical composition can be a drug for treating or preventing a disease selected from immune disorders, infectious diseases, and a combination thereof. In some cases, the pharmaceutical composition can be an adjuvant (Nano-Adjuvant) to enhance an immune response, for example, for a vaccine. In some cases, the pharmaceutical composition can be a prophylactic vaccine, a therapeutic vaccine, or a combination thereof, wherein the pharmaceutical composition comprises the adjuvant (Nano-Adjuvant) and further comprises at least one immune agent for stimulating an immune response in a subject in need thereof.

In some cases, the present invention is directed to a method for treating or preventing a disease of a subject in need thereof, the method comprising administering to the subject an effective dose of a pharmaceutical composition disclosed herein.

### BRIEF DESCRIPTION OF THE FIGURES

FIG. 1A-FIG. 1D. Examples of SBPs including (FIG. 1A) a dendrimer, (FIG. 1B) a dendrigraft, (FIG. 1C) a regular comb-branched polymer and (FIG. 1D) a star-branched polymer. All have a core, either globular or linear.

FIG. 2A and FIG. 2B. Examples of chemical structures of symmetrically branched polypropyleneimine (PPI) dendrimers. FIG. 2A: A dendrimer with 4-PPI. FIG. 2B: A dendrimer with additional 8-PPI.

FIG. 3. Examples of chemical modification reactions of symmetrically branched PPI dendrimers. The numbers, 8, 16, 32, 64, 128 and so on, indicate the number of reactive groups at the surface of the dendrimer.

FIG. 4A and FIG. 4B. Schematic examples of random (FIG. 4A) and regular (FIG. 4B) asymmetrically branched polymers (ABPs) with asymmetric branch junctures and patterns.

FIG. 5. An example of a chemical structure of a random asymmetrically branched PEI homopolymer.

FIG. 6A-FIG. 6C. Examples of synthetic schemes. FIG. 6A: Chemical modification reactions of random asymmetrically branched PEI homopolymers. FIG. 6B: Example of a one-pot synthesis of hydrophobically modified, randomly branched poly(2-ethyloxazoline) with a primary amino group at the focal point of the polymer. The initiator/surface group (I) is a brominated hydrocarbon. The reaction opens the oxazoline ring. FIG. 6C: Non-limiting examples of polymers having different first terminal and second terminal groups.

FIG. 7A and FIG. 7B. Schematic examples of illustrations of a drug loaded in or at the surface domain or region of a branched polymer (FIG. 7A) SBPs and (FIG. 7B) ABPs. In this and other figures, R indicates a surface group and a solid circle depicts a bioactive agent, such as, a drug of interest.

FIG. 8. A schematic illustration of an example of nanoparticles containing both drug molecules (solid circle) and branched polymers with surface groups (R).

FIG. 9A and FIG. 9B. Schematic examples of illustrations of a water insoluble or poorly water soluble drug that is loaded at hydrophobic surface groups of branched polymers (FIG. 9A) SBPs and/or (FIG. 9B) ABPs. In this and other figures, a thin wavy line depicts a hydrophobic surface group.

FIG. 10A and FIG. 10B. Schematic examples of various drug-containing nanoparticles (FIG. 10A) comprising an SBP and (FIG. 10B) comprising an ABP also carrying at least one targeting group or moiety, such as, an antibody, depicted herein and in other figures as a “Y”.

FIG. 11A-FIG. 11D. Formula of examples of STING agonists. FIG. 11A: Formula (1)-(6). FIG. 11B: Formula (7)-(12). FIG. 11C: Formula (13)-(18). FIG. 11D: Formula (19)-(24). FIG. 11E: Formula (25)-(29).

FIG. 12. Examples of receptor binding domain (RBD) antigen constructs with 3 RBD sequences. Linkers are not shown in AG1-AG6.

FIG. 13A-FIG. 13F. Representative schematic illustrations of Examples of expression cassettes and corresponding RBD antigen fusion proteins. FIG. 13A: Phylogenetic tree of some relevant viruses, such as, coronaviruses (CoV). FIG. 13B: Examples of RBD antigen fusion proteins. FIG. 13C: An example of an antigen having 3 RBDs with L15 linkers. FIG. 13D: An example of an antigen having 3 RBDs with L20 linkers. FIG. 13E: An example of an antigen having 2 RBDs. FIG. 13F: Another example of an antigen having 2 RBDs.

FIG. 14. An example of a representative sequence alignment for the RBD sequences from various β-coronaviruses.

FIG. 15A-FIG. 15D. Examples of immune responses in mice. FIG. 15A: Cytokine mRNA levels at 6 hours after nasal vaccination. FIG. 15B: Levels of serum IgG specific to SARS-CoV-2 RBD after two doses of vaccination.

FIG. 15C: Levels of serum IgG specific to SARS-CoV RBD after two doses of vaccination. FIG. 15D: Levels of serum IgG specific to Middle East Respiratory Syndrome (MERS)—CoV RBD after two doses of vaccination.

FIG. 16A-FIG. 16D. Examples of immune responses in mice at 63 days after the first immunization (3 doses). FIG. 16A: Levels of serum IgG specific to SARS-CoV-2 RBD after three doses of vaccination. FIG. 16B: Levels of serum IgA specific to SARS-CoV-2 RBD after three doses of vaccination. FIG. 16C: Levels of BAL IgA specific to SARS-CoV-2 RBD after three doses of vaccination. FIG. 16D: Levels of neutralization antibody specific to SARS-CoV-2 (D614G) RBD after three doses of vaccination with different adjuvants.

FIG. 17A-FIG. 17D. Examples of cellular immune response in Lung and spleen after Intranasal (IN) immunization. FIG. 17A: Data on spleen IFN-γ. FIG. 17B: Data on lung IFN-γ. FIG. 17C: Data on spleen IL-4. FIG. 17D: Data on lung IL-4.

FIG. 18A-FIG. 18C. Examples of comparison of dosage level and IgG immune response with intranasal (IN) and intramuscular (IM) immunization. FIG. 18A: Serum IgG specific to SARS-CoV-2 RBD. FIG. 18B: Serum IgG specific to SARS-CoV RBD. FIG. 18C: Serum IgG specific to MERS-CoV RBD.

FIG. 19A-FIG. 19D. Examples of neutralizing antibody with intranasal (IN) immunization. FIG. 19A: Levels of neutralization antibody specific to SARS-CoV-2 (Strain D614G). FIG. 19B: Levels of neutralization antibody specific to SARS-CoV-2 (Strain BA.5). FIG. 19C: Levels of neutralization antibody specific to SARS-CoV. FIG. 19D: Levels of neutralization antibody specific to MERS-CoV.

FIG. 20A-FIG. 20C. Examples of measurement data of IgG immune response in monkey, rhesus macaques (Macaca mulatta). FIG. 20A: IgG levels in serum. FIG. 20B: IgG levels in BAL. FIG. 20C: IgG levels in NAL.

FIG. 21A-FIG. 21C. Examples of measurement data of neutralization antibody in monkey, rhesus macaques. FIG. 21A: Levels of neutralization antibody specific to SARS-CoV-2 (Strain D614G). FIG. 21B: Levels of neutralization antibody specific to SARS-CoV-2 (Strain Delta). FIG. 21C: Levels of neutralization antibody specific to SARS-CoV-2 (Strain BA.5). FIG. 21D: Levels of neutralization antibody specific to SARS-CoV-2 (Strain BA.2.2). FIG. 21E: Levels of neutralization antibody specific to SARS-CoV. FIG. 21F: Levels of neutralization antibody specific to MERS-CoV.

### DETAILED DESCRIPTION

Features and advantages of the present invention will be more readily understood, by those of ordinary skill in the art, from reading the following detailed description. It is to be appreciated that certain features of the invention, which are described above and below in the context of separate embodiments, may also be provided in combination in a single embodiment. Conversely, various features of the invention that are, for brevity, described in the context of a single embodiment, may also be provided separately or in any combination or sub-combination. In addition, references in the singular may also include the plural (for example, “a” and “an” may refer to one, or one or more) unless the context specifically states otherwise.

Use of numerical values in the various ranges specified in this application, unless expressly indicated otherwise, are stated as approximations as though minimum and maximum values within the stated ranges were both proceeded by the word, “about”. In this manner, slight variations above and below the stated ranges can be used to achieve substantially the same results as values within the ranges. Also, disclosure of ranges is intended as a continuous range including every value between the minimum and maximum values and including the minimum and maximum cited values.

The drug solubility in the instant disclosure is defined as, relative to parts of solvent required to solubilize one part of bioactive agent or drug, <30 (soluble), 30-100 (poorly soluble) and >100 (insoluble). Water solubility is defined herein as, relative to parts of water required to solubilize one part of bioactive agent or drug, <30 (water soluble), 30-100 (poorly water soluble) and >100 (water insoluble).

For the purposes of the instant disclosure, a randomly branched PEI, although there are branches of different length and branches occur randomly, is considered as a homopolymer because that branched polymer is composed of a single monomer, the ethyleneimine or aziridine repeat unit. A polymer having a structure of “(AB)-(AB)-(AB) . . . ” can also be considered as a homopolymer because of the (AB) repeating unit, where A and B are differing monomers. The homopolymer may be linear or branched. Also, one or more of the monomer or complex monomer components can be modified, substituted, derivatized and so on, for example, modified to carry a functional group. Such molecules are homopolymers for the purposes of the instant disclosure as the polymer backbone is composed of a single type of simple or complex monomer.

The term “polymer” refers to any polymer suitable for this invention as defined above and hereafter. In examples, a polymer can comprise polyoxazoline or modified polyoxazoline as disclosed herein. In further examples, the polymer can comprise a modified polyoxazoline can comprise one or more second terminal groups, such as an —NH₂, —NH, —NH₃⁺, other basic groups or a combination thereof, with the proviso that in a range of from 0.01% to 100% of the second terminal group is free from primary amine. In some cases, in a range of from 0.01% to 100%, 0.1% to 100%, or 1% to 100%, the second terminal group is free from primary amine. In some cases, in a range of from 1% to 100% of the second terminal group can comprise a hydroxyl group. All percentages are based on the total number of the second terminal groups.

The term “bioactive agent” or “bioactive agents” refers to a molecule, a compound, a complex of one or more compounds or molecules, or a combination thereof that can provide a biological activity in vivo, in vitro, or a combination thereof. A pharmaceutical composition can comprise one or more bioactive agent, such as, pharmaceutically active agents (PAAs) or active pharmaceutical ingredients (APIs), and other bioactive or inert compounds that can include emollients, antioxidants, such as, astaxanthin, bleaching agents, antiperspirants, pharmaceuticals, moisturizers, scents, colorants, pigments, dyes, antioxidants, oils, fatty acids, lipids, inorganic salts, organic molecules, opacifiers, vitamins, pharmaceuticals, keratolytic agents, UV blocking agents, tanning accelerators, depigmenting agents, deodorants, perfumes, insect repellants, or a combination thereof. Some examples of bioactive agents are described in detail in this disclosure. In some cases, the term “bioactive agent” can comprise a STING polypeptide, a nucleic acid encoding said STING polypeptide, a STING inhibitor, a STING activator, a STING agonist, a STING antagonist, a STING modulating molecule, or a combination thereof.

The term “pharmaceutical suitable carrier”, “pharmaceutical suitable carriers”, “pharmaceutically suitable carrier” or “pharmaceutically suitable carriers” refers to one or more inactive ingredients that are in approved drug products. Inactive ingredients listed in the database “Inactive Ingredients in Approved Drug Products” maintained and updated by US Food and Drug Administration (FDA) can be suitable. In some cases, a pharmaceutically suitable carrier can also be referred to as an excipient.

The term “subject” or “subjects” used throughout this disclosure refers to an animal, a human or a human patient. The term “animal” refers to wild animals, captured or zoo-raised animals and domesticated animals including livestock, farm animals, pets, laboratory animals, such as, horse, cattle, pig, donkey, mule, camel, goat, sheep, monkey, rabbit, dog, cat, mouse, rat, and the like. Warm-blooded animals are suitable. The term “human” refers to a human patient having one or more diseases in need of a treatment, a person having one or more medical conditions unrelated to a treatment, or a healthy person. In some cases, a subject can be a human patient or a healthy person.

The term “antibody”, “antibodies” or “fragment of an antibody” can include natural or synthetic antibodies that selectively bind to an antigen. The term includes polyclonal and monoclonal antibodies produced from animals, cells including eukaryotic or prokaryotic cells, cell free systems, or chemical synthesis. In addition to intact immunoglobulin molecules, also included in the term “antibodies” are fragments or polymers of those immunoglobulin molecules, and human or humanized versions of immunoglobulin molecules that selectively bind a target antigen.

The term “aqueous solution” or “aqueous solutions” used throughout this disclosure refers to a solution comprises in a range of from 80% to 100% water, percentage based on the total non-solid weight of the aqueous solution. An aqueous solution can further comprise additional components, such as salt, acid, base, buffer, solvent, organic solvent, particles, emulsion, solids or non-solids, detergents, small molecules, large molecules, other ingredients, or a combination thereof. The term “non-solid weight” refers to the weight of solid content after the aqueous solution is dried out, such as, by removing all the water or other liquids.

The term “infectious disease” or “infectious diseases” refers to illnesses caused by harmful organisms (pathogens), such as, bacteria, viruses, fungi, protozoa, worms, parasites, prions, a part thereof, or a combination thereof. The infectious diseases can be transmitted among people, from contacting with animals, insects, or from contaminated food, water or soil. Some examples of infectious diseases can include Chickenpox (Varicella), Coronaviruses, Dengue, Diphtheria, Ebola, Flu (Influenza), Hepatitis, Hib Disease, HIV/AIDS, HPV (Human Papillomavirus), Japanese Encephalitis, Measles, Meningococcal Disease, Monkeypox, Mumps, Norovirus, Pneumococcal Disease, Polio, Rabies, Respiratory Syncytial Virus (RSV), Rotavirus, Rubella (German Measles), Shingles (Herpes zoster), Tetanus (Lockjaw), Whooping Cough (Pertussis), Zika, and other known diseases, or diseases that yet to emerge or be identified.

The term “vaccine” or “vaccines” refers to a substance or group of substances that are designed to cause the immune system of a subject, such as, humans or animals to respond to microorganisms, such as, bacteria, viruses, fungi, protozoa, worms, parasites, prions, or other harmful organisms (pathogens). A vaccine can help the body recognize and destroy microorganisms or other pathogen cells. In some cases, a vaccine can comprise a protein, nucleic acids encoding the protein, a toxin, nucleic acids, oligo nucleic acids; DNAs; RNAs; mRNAs; siRNAs; single guide RNAs (sgRNAs); or a combination thereof, from the microorganisms or other pathogen cells. In some cases, a vaccine can comprise a modified protein, nucleic acids encoding the modified protein, a toxin, nucleic acids, modified nucleic acids, oligo nucleic acids, or modified oligo nucleic acids; DNAs; RNAs; mRNAs; siRNAs; sgRNAs; or a combination thereof, that are designed to cause the immune system to respond to the microorganisms or other pathogens. Modified or synthetic DNAs, RNAs, mRNAs, siRNAs, sgRNAs, or a combination thereof, can also be suitable.

The term “adjuvant” or “adjuvants” refers to a drug, substance, a bioactive agent, a reagent, or a combination thereof, that is used to modify, augment, or increase the efficacy or potency of a vaccine to provide better immunity to a particular disease. Adjuvants can comprise one or more organic molecules; antigenic molecules that can mimic specific pathogen-associated molecular patterns, which include liposomes, lipopolysaccharide, molecular cages for antigens, components of bacterial cell walls, and endocytosed nucleic acids such as RNA, double-stranded RNA (dsRNA), DNA, single-stranded DNA (ssDNA), methylated or unmethylated CpG dinucleotide-containing DNA; inorganic compounds, such as, potassium alum, aluminum hydroxide, aluminum phosphate, calcium phosphate hydroxide; oils, such as, paraffin oil propolis, peanut oil; bacterial products, such as, killed bacteria; plant products, such as, those from soybean or other plants; cytokines, such as, IL-1, IL-2 or IL-12; or a combination thereof.

The term, “isomer,” or, “isomers,” refers to molecules that share the same chemical formula but have their atoms connected differently or arranged differently in space, including structural isomers having respective atoms bonded together in different order, geometric isomers having atoms bonded in the same order, but differ in the configuration around the bonds, such as, cis-isomers or trans-isomers and enantiomers having the same chemical structure but differ in three-dimensional arrangement of atoms around asymmetric carbons, such that they are mirror images of one another.

The term “admix”, “ad-mix”, “co-admix”, “admixed”, “co-admixed”, “admixing”, “co-admixing” or a grammatical variation thereof refers to mixing two or more different drug products prior to administration, so only one administration, such as an injection, is performed for the two or more drug products. Admixing can usually be done in a secondary container where the two or more drug products can be mixed together, and can typically be kept for a limited time (usually less than a few days, such as less than 1-3 days, less than 24 hours, less than 8 hours, or less than 4 hours) prior to administration.

The term “coformulation”, “coformulate”, “coformulating”, “co-formulating”, “co-formulated”, “co-formulate”, “co-formulation”, or a grammatical variation thereof refers to two or more drug substances that are formulated together in the same primary packaging (a pill for an oral dose, for example, a vial for an injectable). There is no admixing necessary because the two or more drug substances are already mixed. The two or more drug substances coformulated together can be stored for an extended period of time, such as, 6 months, 12 months, 24 months or longer. One or more pharmaceutical acceptable excipients can be used in a co-formulation.

The term “pre-mix”, “pre-mixed”, “premix”, or “premixed” can refer to “admix”, “admixing”, “admixed”, “coformulate”, “coformulated”, “coformulating”, “co-formulate”, “co-formulated”, “co-formulating”, or a combination thereof.

The term “variant”, “variants”, “variant thereof” or “variants thereof” used herein throughout this disclosure means a variant or a non-identical form for example, of a pathogen or a part thereof. In some cases, the term can refer to a variant protein, DNA or RNA of a pathogen. In some cases, the term refers to variants of the coronavirus. In some cases, a variant can comprise a variant of the spike glycoprotein (S protein) or a part thereof, a variant of the spike glycoprotein receptor-binding domain (RBD) polypeptide or a part thereof, a variant of the membrane protein (M protein) or a part thereof, a variant of the envelope protein (E protein) or part thereof, of a variant coronavirus. In some cases, a variant can comprise a variant identified in the U.K. (B.1.1.7) (SARS-CoV-2a with N501Y mutation), a variant identified in South Africa (B.1.351) (SARS-CoV-2β with K417N, E484K and N501Y mutations), a variant identified in Japan/Brazil (P.1) (SARS-CoV-2γ with K417T, E484K and N501Y mutations), a variant identified in India (SARS-CoV-20), a variant identified in the U.S. (California) (B.1.427), a variant identified in the U.S. (California) (B.1.429), B.1.525 variant (first identified in United Kingdom/Nigeria), B.1.526 (first identified in United States (New York), B.1.526.1, B.1.617 (first identified in India with L452R, E484Q, D614G mutations), B.1.617.1 (first identified in India with at least 154K mutation and mutations at other positions), B.1.617.3 (first identified in India with T19R, G142D, L452R, E484Q, D614G, P681R, D950N mutations), P.2 (first identified in Brazil with at least E484K, D614G, V1176F mutations), or other variants of the coronavirus derived from SARS-CoV-2, as published by the US CDC (https://www.cdc.gov/coronavirus/2019-ncov/variants/variant-info.html). In some cases, the variant can comprise mutations, deletions, or a combination thereof in the S protein, the N protein, the E protein, or the M protein. In some cases, the variant can comprise one or more mutations at one or more of the 417, 484, 501, 677 or 681 positions of the S protein, 85 position of the M protein, 377 position of the N protein, or a combination thereof. In some cases, the variant can comprise E501Y, E484K or a combination thereof, of the S protein.

In some cases, this disclosure is directed to a pharmaceutical composition comprising:
a nanoaggregate comprising a polymer and at least one bioactive agent comprising at least one STING polypeptide or a part thereof, a nucleic acid encoding a STING polypeptide or a part thereof, a STING inhibitor, a STING activator, a STING agonist, a STING antagonist, a STING modulating molecule, or a combination thereof; and
optionally a pharmaceutical suitable carrier;
wherein the pharmaceutical composition is soluble in an aqueous solution to produce at least 1 mg/ml of the bioactive agent in the aqueous solution;
wherein the polymer is water soluble; and
wherein the polymer comprises:
a first polymer comprising at least one first terminal group modified with H or a hydrophobic moiety and a second terminal group modified with a hydrophilic moiety, wherein the first terminal group comprises in a range of from 1% to 100% of H and 0% to 99% of the hydrophobic moiety that comprises a saturated or an unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof;
a second polymer; or
a combination thereof.

In some cases, the polymer can comprise a first polymer, as disclosed herein comprising at least one first terminal group modified with H or a hydrophobic moiety and a second terminal group modified with a hydrophilic moiety, and wherein the first terminal group comprises in a range of from 1% to 100% of H and 0% to 99% of the hydrophobic moiety that can comprise a saturated or an unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof.

In some cases, the polymer can consist of a first polymer, as disclosed herein comprising at least one first terminal group modified with H or a hydrophobic moiety and a second terminal group modified with a hydrophilic moiety, and wherein the first terminal group comprises in a range of from 1% to 100% of H and 0% to 99% of the hydrophobic moiety that can comprise a saturated or an unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof. In some cases, the pharmaceutical composition can be free from polymers selected from one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof. In embodiments, the first terminal group comprises a range from 1% to 100% H and from 0% to 99% of hydrophobic moiety.

In some cases, the polymer can comprise a second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or a combination thereof.

In some cases, the polymer can consist of one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof. In some cases, the polymer can comprise only the polymers listed above and be free from the aforementioned polymers comprising the at least one first terminal group modified with H or a hydrophobic moiety and the second terminal group modified with a hydrophilic moiety.

In some cases, the polymer can comprise a first polymer and one or more subsequent polymers (also referred to as the “second polymer”) selected from one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof.

In some cases, the polymer can comprise a polyoxazoline (POX) that comprises a linear portion, a branched portion, or a combination thereof, and wherein the polyoxazoline (POX) can comprises poly(2-methyloxazoline), poly(2-ethyloxazoline), poly(2-propyloxazoline), poly(isopropyloxazoline), or a combination thereof. In some cases, the polyoxazoline can be poly(2-ethyloxazoline) (PEOX).

In some cases, the polyoxazoline can comprise a molar ratio of monomer to initiator in a range of from 50:1 to 80:1.

In some cases, in a range of from 1% to 100% of the second terminal group is free from primary amine. In some cases, the pharmaceutical composition disclosed herein, in a range of from 1% to 100% of the second terminal group can comprise a hydroxyl group. All percentages are based on the total number of the second terminal groups.

In some cases, the first terminal group comprises in a range of from 1% to 100% of H and 0% to 99% hydrophobic moiety, from 1% to 99% of H and 1% to 99% of the hydrophobic moiety, 1% to 90% of H and 10% to 99% of the hydrophobic moiety, 1% to 85% of H and 15% to 99% of the hydrophobic moiety, 1% to 80% of H and 20% to 99% of the hydrophobic moiety, 1% to 75% of H and 25% to 99% of the hydrophobic moiety, 1% to 70% of H and 30% to 99% of the hydrophobic moiety, 1% to 65% of H and 35% to 99% of the hydrophobic moiety, 1% to 60% of H and 40% to 99% of the hydrophobic moiety, 1% to 55% of H and 45% to 99% of the hydrophobic moiety, 1% to 50% of H and 50% to 99% of the hydrophobic moiety, 1% to 45% of H and 55% to 99% of the hydrophobic moiety, 1% to 40% of H and 60% to 99% of the hydrophobic moiety, 1% to 35% of H and 65% to 99% of the hydrophobic moiety, 1% to 30% of H and 70% to 99% of the hydrophobic moiety, 1% to 25% of H and 75% to 99% of the hydrophobic moiety, 1% to 20% of H and 80% to 99% of the hydrophobic moiety, 1% to 15% of H and 85% to 99% of the hydrophobic moiety, 1% to 10% of H and 90% to 99% of the hydrophobic moiety, 1% to 5% of H and 95% to 99% of the hydrophobic moiety, 1% of H and 99% of the hydrophobic moiety, including all percentages within the range, the percentages based on the total number of the first terminal group in the polymer. In some cases, the first terminal group comprises in a range of from 1% to 50% of H and 50% to 99% of the hydrophobic moiety, 1% to 40% of H and 60% to 99% of the hydrophobic moiety, 1% to 30% of H and 70% to 99% of the hydrophobic moiety, 1% to 20% of H and 80% to 99% of the hydrophobic moiety, 1% to 10% of H and 90% to 99% of the hydrophobic moiety, 1% to 5% of H and 95% to 99% of the hydrophobic moiety, or 1% to 2% of H and 98% to 99% of the hydrophobic moiety, including all percentages within the range, the percentages based on the total number of the first terminal groups in the polymer. In some cases, the percentage is based on molar numbers of the first terminal groups in the polymer.

Alternatively, an H to hydrocarbon group (“hydrocarbon”) ratio can be used to describe the polymer, such as H:hydrocarbon=0.01:1 to 100:1. In some cases, the first terminal group comprises a ratio of H:hydrophobic moiety in a range of from 0.01:1 to 100:1, including all ratios within the range. In some cases, the first terminal group comprises a ratio of H:hydrophobic moiety in a range of from 0.01:1 to 100:1, 0.1:1 to 100:1, 0.2:1 to 100:1, 0.5:1 to 100:1, 0.7:1 to 100:1, 1:1 to 100:1, 2.0:1 to 100:1, 5;1 to 100:1, 10:1 to 100:1, 20:1 to 100:1, 30:1 to 100:1, 40:1 to 100:1, 50:1 to 100:1, 60:1 to 100:1, 70:1 to 100:1, 80:1 to 100:1, 90:1 to 100:1, and 95:1 to 100:1, including all ratios within the range. In some cases, the first terminal group comprises a ratio of H:hydrophobic moiety in a range of from 0.01:1 to 10:1, 0.1:1 to 10:1, 0.1:1 to 10:1, 0.2:1 to 10:1, 0.5:1 to 10:1, 0.7:1 to 10:1, 1:1 to 10:1, 2.0:1 to 10:1, 5;1 to 10:1, 10:1, 20:1 to 10:1, 30:1 to 10:1, 40:1 to 10:1, 50:1 to 10:1, 60:1 to 10:1, 70:1 to 10:1, 80:1 to 10:1, 90:1 to 10:1, and 95:1 to 10:1, including all ratios within the range. In some cases, the first terminal group comprises a ratio of H:hydrophobic moiety in a range of from 0.01:1 to 5:1, 0.1:1 to 5:1, 0.1:1 to 5:1, 0.2:1 to 5:1, 0.5:1 to 5:1, 0.7:1 to 5:1, 1:1 to 5:1, 2.0:1 to 5:1, 5;1, 10:1, 20:1 to 5:1, 30:1 to 5:1, 40:1 to 5:1, 50:1 to 5:1, 60:1 to 5:1, 70:1 to 5:1, 80:1 to 5:1, 90:1 to 5:1, and 95:1 to 5:1, including all ratios within the range. In some cases, the first terminal group comprises a ratio of H:hydrophobic moiety can be selected from 0.01:1, 0.1:1, 0.2:1, 0.5:1, 0.7:1, 1:1, 2.0:1, 3.0:1, 4.0:1, 5;1, 6;1, 7:1, 8:1, 9:1, 10:1, 20:1, 30:1, 40:1, 50:1, 60:1, 70:1, 80:1, 90:1, 95:1, and 100:1, including all ratios within the range. The ratio can be based on molar ratio of the H and the hydrocarbon group.

The percentage and ratio can be converted by a conventional method, for example, a ratio of 0.01:1 can be converted to about 1%, 0.2:1 can be converted to about 17%, 0.5:1 can be converted to about 33%, 1:1 can be converted to about 50%, 1.5:1 can be converted to about 60%, 2:1 can be converted to about 67%, 5:1 can be converted to about 83%, 10:1 can be converted to about 90%, 20:1 can be converted to about 95%, and 100:1 can be converted to about 99%.

The percentage or the ratio of the hydrogen modified first terminal group and the hydrocarbon modified first terminal group can be measured with HPLC as known to those skilled in the art.

In the pharmaceutical composition disclosed herein, the first terminal group can comprise H or a hydrophobic moiety that can comprise a saturated or an unsaturated aliphatic hydrocarbon having 1 to about 22 carbons, an aromatic hydrocarbon, or a combination thereof, and the second terminal group can comprise a group modified by an amine, amide, imine, imide, carboxyl, hydroxyl, ester, ether, acetate, phosphate, ketone, aldehyde, sulfonate, or a combination thereof.

The first terminal group can comprise hydrogen (H) in one example, a hydrocarbon having 2 to 22 carbons in one example, 4 to 22 carbons in another example, 6 to 22 carbons in yet another example, 7 to 22 carbons in yet another example, 8 to 22 carbons in yet another example, 10 to 22 carbons in yet another example, 12 to 22 carbons in yet another example, 14 to 22 carbons in yet another example, 16 to 22 carbons in yet another example, and 18 to 22 carbons in a further example. In one particular example, the first terminal group can comprise 18 carbons, such as, a (CH₃(CH₂)₁₇)-group. In some cases, the first terminal group can comprise a hydrocarbon having 7 to 22 carbons. In some cases, the first terminal group can comprise H. In some cases, the first terminal group can comprise in a range of from 1% to 100% of H and 0% to 99% of the hydrophobic moiety that can comprise a saturated or an unsaturated aliphatic hydrocarbon having 1 to about 22 carbons, an aromatic hydrocarbon, or a combination thereof. In embodiments, the first terminal group comprises a range from 1% to 100% H and from 0% to 99% of hydrophobic moiety. The first terminal group can be modified by selecting various initiators. In some cases, p-toluenesulfonic acid, trifluoroacetic acid, methyl tosylate, HCl, HBr, Hl, H—Br, hydrocarbon-Br such as C₁ to C₂₂-Br, or a combination thereof, can be utilized as an initiator. Polymers prepared herein can be mixed together at pre-determined ratios.

The initiator can comprise a hydrophobic electrophilic molecule, including hydrocarbons, aliphatic hydrocarbons, aromatic hydrocarbons or a combination thereof, along with a halide functional group, such as, alkyl halides, aralkyl halides, acyl halides or combinations thereof. Examples of such compounds can include monofunctional initiators, such as, hydrocarbons containing from 1 to about 22 hydrocarbons with either saturated or unsaturated chemical bonds, such as, methyl iodide/bromide/chloride, ethyl iodide/bromide/chloride, 1-iodo/bromo/chloro butane, 1-iodo/bromo/chloro hexane, 1-iodo/bromo/chloro dodecane, 1-iodo/bromo/chloro octadodecane, benzyl iodide/bromide/chloride and so on. Other initiators can include allyl bromides/chlorides. Acyl halides, such as, acyl bromide/chloride, benzoyl bromide/chloride and tosyl group-containing compounds, such as, p-toluenesulfonic acid, methyl tosylate and other tosylate esters can also be used. Any one or more initiators can be used in combination. In some cases, the initiator can also comprise a hydrophilic moiety comprising proton/H containing molecules, such as, p-toluenesulfonic acid, trifluoroacetic acid, methyl tosylate, HCl, HBr, Hl, or a combination thereof.

During polymerization, an initiator can be used to start polymerization. When used, various molar ratios of monomer to initiator can be used to obtain particular polymers. The particular polymers can have differing properties, such as, molecular weight, size of branching and other properties including those unexpectedly discovered by Applicants as disclosed herein. In some cases, suitable monomer to initiator molar ratios can be 20:1 to 100:1 including any and all ratios within the range, such as, 20:1, 25:1, 30:1, 35:1, 40:1, 45:1, 50:1, 55:1, 60:1, 65:1, 70:1, 75:1, 80:1, 85:1, 90:1, 95:1 and 100:1 including 20;1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54;1, 55:1, 56:1, 57:1, 58:1, 59:1, 60:1, 61:1, 62:1, 63:1, 64:1, 65:1, 66:1. 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, and so on, meaning that a molar ratio of monomer to initiator in a range specified above is used to produce a polymer of choice. In some cases, a polyoxazoline disclosed herein can comprise a molar ratio of monomer to initiator in a range of from 50:1 to 80:1, meaning that a molar ratio of monomer to initiator in a range of from 50:1 to 80:1, including any and all ratios within the range, can be used to produce a polymer of choice.

The polymer can be prepared with monomers and an initiator as described herein and in PCT Publication No. WO2014/123791, herein incorporated by reference in entirety.

Hydrogen modified randomly branched PEOX polymer having a certain monomer to initiator molar ratio in a range of from 20:1 to 100:1 can be prepared as described above with an initiator selected from a hydrophilic moiety comprising proton/H containing molecules, such as, p-toluenesulfonic acid, trifluoroacetic acid, methyl tosylate, HCl, HBr, Hl, or a combination thereof.

Hydrocarbon C₁ to (CH₃(CH₂)₂₁)-modified randomly branched PEOX polymer having a monomer to initiator molar ratio in a range of from 20:1 to 100:1 can be prepared as described above with an initiator selected from CH₃—Br, (CH₃(CH₂))—Br, (CH₃(CH₂)₂)—Br, (CH₃(CH₂)₃)—Br, (CH₃(CH₂)₄)—Br, (CH₃(CH₂)₅)—Br, (CH₃(CH₂)₆)—Br, (CH₃(CH₂)₇)—Br, (CH₃(CH₂)₈)—Br, (CH₃(CH₂)₉)—Br, (CH₃(CH₂)₁₀)—Br, (CH₃(CH₂)₁₂)—Br, (CH₃(CH₂)₁₂)—Br (CH₃(CH₂)₁₃)—Br, (CH₃(CH₂)₁₄)—Br, (CH₃(CH₂)₁₅)—Br, (CH₃(CH₂)₁₆)—Br, (CH₃(CH₂)₁₇)—Br, (CH₃(CH₂)₁₈)—Br, (CH₃(CH₂)₁₉)—Br, (CH₃(CH₂)₂₀)—Br, and (CH₃(CH₂)₂₁)—Br. A mixture of the initiators can also be suitable.

In some cases, a polymer comprising a mixture of hydrocarbon, such as, C₁ to (CH₃(CH₂)₂₁)-modified first terminal group and H modified first terminal group can be produced by mixing a hydrogen modified randomly branched PEOX polymer and a hydrocarbon C₁ to (CH₃(CH₂)₂₁)-modified randomly branched PEOX polymer prepared above at a predetermined ratio. In some cases, a polymer can comprise in a range of from 1% to 99% of the hydrocarbon, such as, C₁ to (CH₃(CH₂)₂₁)-modified first terminal group and in a range of from 1% to 99% of H modified first terminal group.

In some cases, the first terminal group can comprise in a ratio of H to hydrophobic moiety having a C₁ to C₂₂ hydrocarbon, such as, (CH₃(CH₂)₁₇)-in a range of from 0.01:1 to 100:1. In some cases, the first terminal group can comprise a ratio of H to hydrophobic moiety having C₁ to C₂₂ hydrocarbon, such as, (CH₃(CH₂)₁₇)—, in a range of from 0.1:1 to 5:1.

Polymers comprising a mixture of hydrocarbon (CH₃(CH₂)₁₇)-modified first terminals and H modified first terminals of a PEOX can be referred to as “H/C₁₈PEOXABP”. Polymers having a specific initiator molar ratio, such as, 60:1, 70:1, 80:1, and so on, can be referred to as “H/C₁₈PEOXABP60”, “H/C₁₈PEOXABP70”, “H/C₁₈PEOXABP80”, and so on, respectively.

The polymer disclosed above and hereafter can be suitable and can comprise a linear polymer, a branched polymer, a symmetrically branched polymer, an asymmetrically branched polymer, a dendrimer, a dendrigraft polymer, a comb-branched polymer, a star-branched polymer, or a combination thereof. The polymer is water soluble. In examples, the polymer can be dissolved in water to produce a 12% weight percent or higher water solution.

The second terminal group can comprise a group modified by an ammonia, a derivative of ammonia, an ethylenediamine (EDA), a derivative of ethylenediamine, a piperazine, a derivative of piperazine, tris(2 aminoethyl)amine, 4-(aminomethyl) piperidine, 1,3-diaminopropane, 2,2′-(ethylenedioxy)bis(ethylamine), diethylenetriamine, 1,4,7,10-tetraazacyclododecane, hexamethylenediamine, triethylenetetramine, 1,8-diaminooctane, or a combination thereof. In yet another example, the second terminal group can comprise a group modified by an ethylenediamine (EDA), a derivative of ethylenediamine, or a combination thereof. Any derivative of ethylenediamine disclosed herein can be suitable. The polymer can have a reaction challenge molar ratio of, for example, polyoxazoline reactive chain end to EDA in a range of from 1:1 to 1:100. The polymer can have a reaction challenge molar ratio of polyoxazoline reactive chain end to EDA in a range of from 1:1 to 1:100 in one example, 1:2 to 1:100 in another example, 1:2 to 1:50 in yet another example, 1:2 to 1:40 in yet another example, 1:2 to 1:30 in a further example, 1:2 to 1:20 in yet another example, 1:2 to 1:15 in yet another example, and 1:5 to 1:15 in a further example. In further examples, a polymer can have a reaction challenge molar ratio of polyoxazoline reactive chain end to EDA at a ratio of about 1:10. The EDA modified polyoxazoline disclosed herein can provide functional groups that can have pH-dependent changes in polymer charge as disclosed herein. In some cases, a pharmaceutical composition disclosed herein can comprise a polymer that can have a molar ratio of polyoxazoline reactive chain end to EDA of about 1:10. In some cases, in a range of from 1% to 99%, 1% to 90%, 1% to 80%, 1% to 70%, 1% to 60%, 1% to 50%, 1% to 40%, 1% to 30%, 1% to 20%, 1% to 10%, 1% to 5%, 1% to 4%, 1% to 3%, 1% to 2%, of the second terminal group can comprise a group modified by EDA. In some cases, in a range of from 1% to 99%, 1% to 90%, 1% to 80%, 1% to 70%, 1% to 60%, 1% to 50%, 1% to 40%, 1% to 30%, 1% to 20%, 1% to 10%, 1% to 5%, 1% to 4%, 1% to 3%, 1% to 2%, of the second terminal group can comprise a primary amine.

In some cases, the second terminal group can comprise a group modified by an amine, amide, imine, imide, carboxyl, hydroxyl, ester, ether, acetate, phosphate, ketone, aldehyde, sulfonate, or a combination thereof, with a proviso that in a range of from 0.01% to 100%, 0.1% to 100%, 1% to 100%, 5% to 100%, 10% to 100%, 15% to 100%, 20% to 100%, 30% to 100%, 40% to 100%, 50% to 100%, 60% to 100%, 70% to 100%, 80% to 100%, 90% to 100%, 95% to 100%, 99% to 100%, the second terminal group can be free from primary amine. In some cases, about 1%, 5%, 10%, 15%, 20%, 25%, 30%, 35%, 40%, 45%, 50%, 55%, 60%, 65%, 70%, 75%, 80%, 85%, 90%, 95% or 100%, of the second terminal group can be free from primary amine. In some cases, about 50% to 100% of the second terminal group can be free from primary amine. In some cases, about 75% to 100% of the second terminal group can be free from primary amine. Yet in some cases, about 90% to 100% of the second terminal group can be free from primary amine.

In some cases, 100% of the second terminal group of the polymer can comprise a group modified with a hydroxyl group. In some cases, as disclosed herein, CH₃(CH₂)₁₇—Br can be utilized as an initiator for 2-ethyloxazoline polymerization through a cationic ring opening process to generate a randomly branched polymer, followed by, for example, dissolving the randomly branched polymer in water to produce a second terminal modified by a hydroxyl group. In some cases, an initiator selected from one comprising a hydrophilic moiety comprises proton/H containing molecules, such as, p-toluenesulfonic acid, trifluoroacetic acid, methyl tosylate, HCl, HBr, Hl, or a combination thereof, can be utilized as an initiator for 2-ethyloxazoline polymerization through a cationic ring opening process to generate a randomly branched polymer, followed by, for example, dissolving the randomly branched polymer in water to produce a second terminal modified by a hydroxyl group. In some cases, about 100% of the second terminal group can comprise a hydroxyl group. In some cases, about 100% of the second terminal group can be free from a primary amine.

The pharmaceutical composition can have a pH value in a range of from about 3.0 to about 10.0. In some cases, the pharmaceutical composition can have a pH value in a range of from about 7.0 to about 9.0. In some cases, the pharmaceutical composition can have a pH value in a range of from about 7.0 to about 8.0. In some cases, the pharmaceutical composition can have a pH value in a range of from about 7.0 to about 7.5. In some cases, the pharmaceutical composition can have a pH value in a range of from about 3.0 to about 6.9, or about 4.0 to about 6.9. In some cases, the pharmaceutical composition can have a pH value in a range of from about 4.0 to about 7.0. In some cases, the pharmaceutical composition can have a pH value in a range of from about 4.0 to about 7.5. In some cases, the pharmaceutical composition can have a pH value in a range of from about 5.6 to about 6.9. The pharmaceutical composition can be adjusted with an acid or a base to arrive at the desired pH range. An acid, such as, HCl or other acids can be suitable. A base, such as, NaOH, or other bases, can be suitable.

In some cases, the pharmaceutical composition can have a pH value in a range of from about 3.0 to about 10.0, and wherein in a range of from 1% to 100% of the second terminal group is free from a primary amine. In some cases, the pharmaceutical composition can have a pH value in a range of from about 5.6 to about 6.9, and wherein about 100% of the second terminal group is free from a primary amine, i.e., 0% of the second terminal group contains a primary amine. In some cases, in a range of from 1% to 100% of the second terminal group can comprise a hydroxyl group, the percentage based on the total number of the second terminal groups.

Polymer H/C₁₈PEOXABP having a hydroxyl group as the second terminal group can be referred to as H/C₁₈PEOXABP-OH. Polymers having a specific initiator molar ratio, such as, 60:1, 70:1, 80:1, and so on, can be referred to as “H/C₁₈PEOXABP60-OH”, “H/C₁₈PEOXABP70-OH”, “H/C₁₈PEOXABP80-OH”, and so on, respectively. Polymer H/C₁₈PEOXABP having an amine group as the second terminal group can be referred to as H/C₁₈PEOXABP-NH₂. Polymers having a specific initiator molar ratio, such as, 60:1, 70:1, 80:1, and so on, can be referred to as “H/C₁₈PEOXABP60-NH₂”, “H/C₁₈PEOXABP70-NH₂”, “H/C₁₈PEOXABP80-NH₂”, and so on, respectively. In some cases, mixtures of the polymers disclosed herein can be suitable.

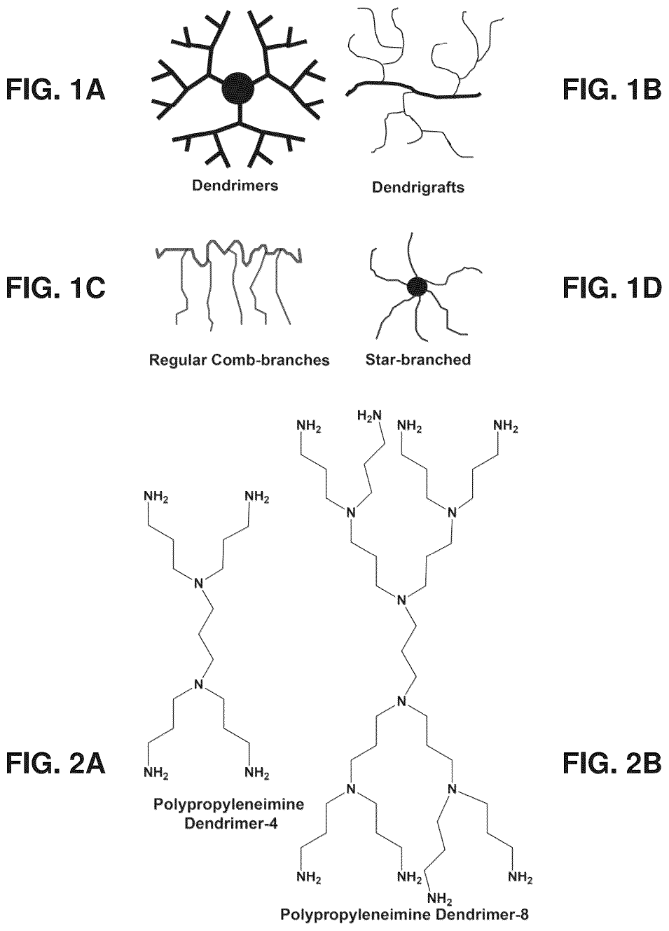

In any of pharmaceutical compositions disclosed above and hereafter, the polymer can comprise a polyoxazoline (POX) that comprises a linear portion, a branched portion, or a combination thereof. The polymer can comprise a plurality of linear portions joined together in one example, one or more linear portions joined with one or more branched portions in another example, one or more branched portions joined together in yet another example, such as, those schematically depicted in FIG. 1A through FIG. 10B. Each of the linear portions can be independently of various lengths, modifications, or a combination thereof. Each of the branched portions can be independently of various lengths, number of branches, modifications, or a combination thereof.

The polyoxazoline (POX) can comprise poly(2-oxazoline), poly(2-substituted oxazoline) that comprises poly(2-methyloxazoline), poly(2-ethyloxazoline), poly(2-propyloxazoline), poly(isopropyloxazoline) (PiPOX), or a combination thereof. The POX can comprise poly(2-methyloxazoline) (PMOX) in one example, poly(2-ethyloxazoline) (PEOX) in another example, poly(2-propyloxazoline) (PPOX) in yet another example, poly(isopropyloxazoline) (PiPOX) in yet another example, or a combination of two or more of the poly(2-substituted oxazoline) s in yet a further example, wherein the two or more of the poly(2-substituted oxazoline) s can be a repeating unit, also referred to as a complex monomer, in the polyoxazoline polymer. The polyoxazoline (POX) is hydrophilic. The polyoxazoline (POX) can be free from monomers, either simple or complex monomers, having hydrophobic side chains, such as, those having 4 or more carbons (C₄ and above).

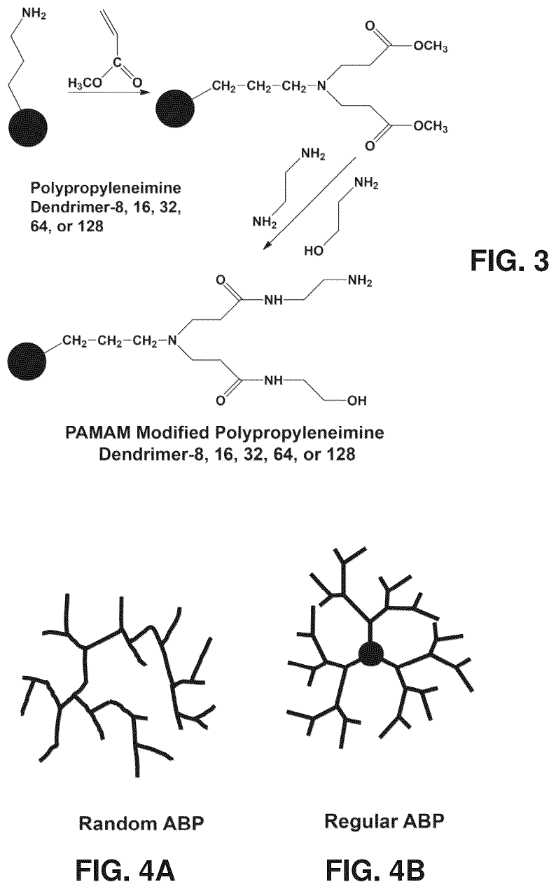

Some of examples of symmetrically branched polymers (SBP) are schematically depicted in FIG. 1A-FIG. 1D and FIG. 2A-FIG. 2B with symmetric branches, wherein all the homopolymers of interest possess a core and exhibit symmetric branch junctures consisting either of terminal or chain branches throughout the homopolymer. The functional groups are present predominantly at the exterior of the polymer.

The modified SBPs can be obtained, for example, through chemically linking functional groups on, for example, symmetrically branched PAMAM or PPI dendrimers, commercially available from Aldrich, polyether dendrimers, polyester dendrimers, comb-branched/star-branched polymers, such as, those containing PEO, PEG, PMOX or PEOX; polystyrene, and comb-branched dendrigrafts, such as, those containing PEOX, PMOX or PEI. The synthetic procedures for making such SBP's/dendrimers are known and as described above and hereafter.

In some cases, the higher branching densities of SBPs can render the polymers molecularly compact with a well-defined interior void space, which makes such molecules suitable as a carrier for water insoluble or poorly water soluble drugs, such as, one or more bioactive agents, entrapped or encased, therein.

The surface modifications can enhance properties and uses of the resulting modified SBPs. For example, with suitable modification, a water insoluble SBP can become water soluble, while an SBP with a high charge density can be modified to carry low or no charge on the polymer or at the polymer surface. On the other hand, a water soluble SBP can be modified with hydrophobic surface groups to enhance ability to solubilize water insoluble or poorly water soluble drugs at the surface or in the interior thereof. Modification can occur at any site of a polymer, for example, at a terminus, a branch, a backbone residue and so on.

In one embodiment of the instant disclosure, the SBP (for example, either a symmetrically branched PEI dendrimer, a PPI dendrimer, a PAMAM dendrimer or a symmetrically branched PEI dendrigraft) can be modified with different kinds of, for example, primary amine groups through, for example, Michael addition or an addition of acrylic esters onto amine groups of the homopolymer. Thus, for example, through a Michael addition reaction, methyl acrylate can be introduced onto primary and/or secondary amino groups of PEI, PPI and polylysine (PLL) homopolymers. The ester groups then can be derivatized further, for example, by an amidation reaction. Thus, for example, such an amidation reaction with, for example, ethylenediamine (EDA), can yield the addition of an amino group at the terminus of the newly formed branch. Other modifications to the homopolymer can be made using known chemistries, for example, as provided in “Poly(amines) and Poly(ammonium salts),” in “Handbook of Polymer Synthesis,” (Part A), Kricheldorf ed., New York, Marcel Dekker, 1994; and “Dendrimers and Other Dendritic Polymers” Frechet & Tomalia, eds., John Wiley & Sons, Ltd., 2001. Derivatives of EDA also can be used and include any molecular entity that comprises a reactive EDA, a substituted EDA or, for example, other members of the polyethylene amine family, such as, diethylenetriamine, triethylenetetramine, tetraethylenepentamine, pentaethylenehexamine, and so on including polyethylene amine, tetramethylethylenediamine and so on. The amidation reaction with, for example, ethylenediamine (EDA), can also modify polymer charge density at the terminus of the newly formed branch. As disclosed herein, polymer having such amidation groups can have pH-dependent change in charge leading to change in pH-dependent polymer charge density.

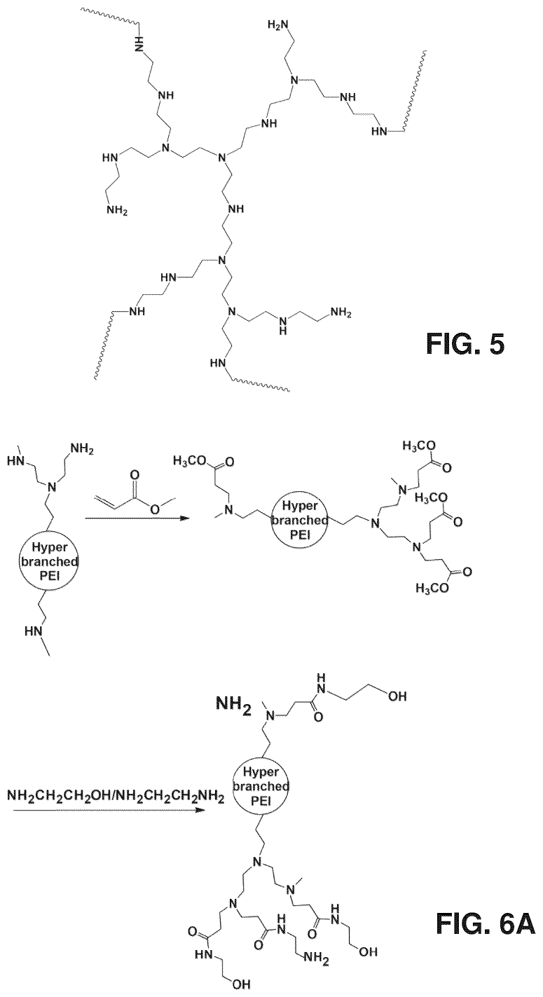

In some embodiments, a modification can comprise a moiety that contributes to or enhances hydrophobicity of a polymer or a portion thereof. For example, hydrophobic functional groups, such as, aliphatic chains including hydrocarbon chains comprising 1 to about 22 carbons that can be saturated or unsaturated, linear, cyclic or branched, aromatic structures (e.g. containing one or more aromatic rings, which may be fused) or combinations thereof, can be used as a modifying agent and added to a polymer as taught herein practicing chemistries as provided herein. On such addition, a modified SBP, such as, a modified PEI, PPI, PAMAM dendrimer or PEI dendrigraft, can be formed. An example of a PAMAM modified PPI dendrimer is shown in FIG. 3. As an extension of the SBP, such as, PPI and PEI, the resulting modified SBP also is symmetrically branched. Depending on the solvent environment (i.e. pH or polarity), the surface functional groups can carry different charge and/or charge density, and/or hydrophobic groups. The molecular shape and surface functional group location (i.e., surface functional group back folding) then can be tuned further, based on those characteristic properties.

In another embodiment of the disclosure, the modified SBPs can be produced using any of a variety of synthetic schemes that, for example, are known to be amenable to reaction with a suitable site on the homopolymer. Moreover, any of a variety of reagents can be used in a synthetic scheme of choice to yield any of a variety of modifications or additions to the homopolymer backbone. Thus, for example, in the case of the Michael addition reaction to an amine described above, the addition of any of a variety of substituents can be used, for example, at the alkylation stage, using, for example, any of a variety of acrylate reagents, such as, an acrylate comprising a hydrocarbon substituent, such as, saturated or unsaturated hydrocarbons comprising 1 to about 22 carbons, which may be substituted, aliphatic, aromatic, ringed, saturated at one or more bonds or a combination thereof. Thus, suitable reactants include, methyl acrylate, ethyl acrylate, propyl acrylate, butyl acrylate, pentyl acrylate, hexyl acrylate, heptyl acrylate, octyl acrylate, nonyl acrylate, decyl acrylate, undecyl acrylate, dodecyl acrylate and so on, and mixtures thereof. Similarly, at the amidation stage in the example exemplified above, any of a variety of amines can be used. For example, EDA, monoethanolamine, tris(hydroxymethyl)aminomethane, alkyl amine, allyl amine or any amino-modified polymer, including those comprising PEG, PEO, perfluoropolymers, polystyrene, polyethylene, polydimethylsiloxane, polyacrylate, polymethylmethacrylate and the like, and mixtures thereof, can be used.

Such a synthetic strategy would allow not only symmetric growth of the molecule, where more branches with different chemical compositions can be introduced, but also addition of multiple functional groups at the exterior of the polymer structure. The precursor homopolymer can be modified, and continuously, using the same or a different synthetic process until the desired SBPs with appropriate molecular weight and functional groups are attained. In addition, the hydrophobic and hydrophilic properties, as well as, charge density of such polymers, can be tailored to fit specific application needs using appropriate monomers for constructing the homopolymer and suitable modification reactions.

In another embodiment of the disclosure, if a divergent synthetic procedure is used, the chain end of a symmetrically star-branched or comb-branched homopolymer, such as, poly(2-oxazoline) or poly(2-substituted oxazoline), including, for example, poly(2-methyloxazoline), poly(2-ethyloxazoline), poly(2-propyloxazoline) and poly(2-butyloxazoline, etc.), PEI, PEO/glycol, polyvinylpyrrolidone (PVP), polyphosphate, polyvinyl alcohol (PVA) or polystyrene, can be modified with another small molecule or polymer to generate various functional groups at the homopolymeric chain ends including a primary, secondary or tertiary amine, carboxylate, hydroxyl, aliphatic (e.g., hydrocarbon chain), aromatic, fluoroalkyl, aryl, PEG, PEO, acetate, amide and/or ester groups. Alternatively, various initiators also can be utilized so that the same type of functional groups can be introduced at the chain end if a convergent synthetic approach is utilized (“Dendritic Molecules,” Newkome et al., eds., VCH, Weinheim, 1996; “Dendrimers and Other Dendritic Polymers,” Frechet & Tomalia, eds., John Wiley & Sons, Ltd., 2001; and J. Macromol. Sci. Chem. A9 (5), pp. 703-727 (1975)).

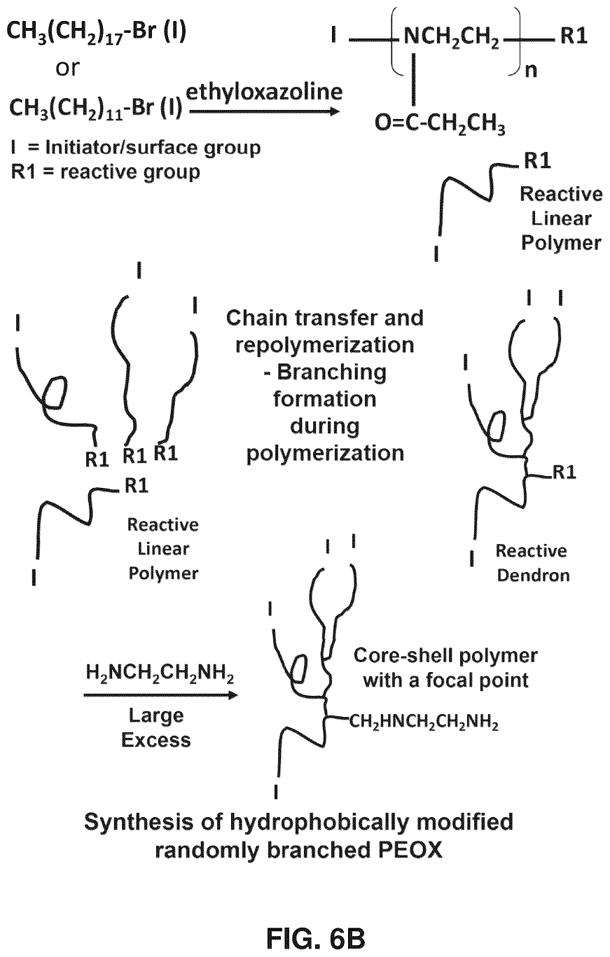

Some examples of asymmetrically branched polymers (ABP) are schematically depicted in FIG. 4A-FIG. 4B with asymmetric branches, wherein some of the polymers of interest possess no core and exhibit asymmetrical branch junctures consisting of both chain and terminal branches throughout the entire homopolymer. The junctional groups often are present both at the exterior and in the interior. However, when a larger functional group (e.g., a large hydrophobic or hydrophilic group) is used, the functional groups often can be attached preferentially and perhaps necessarily at the exterior of the ABP, for example, possibly due to steric effects. Therefore, such surface modified branched polymers (MBP) can be utilized for solubilization of or nanoaggregate formation with a water insoluble or poorly water soluble drug.

The modified ABPs can be obtained, for example, through chemically linking functional groups on regular ABPs, such as, polylysine (e.g., branched PLL), on random ABPs, such as, PEIs (commercially available from Aldrich, Polysciences, or BASF under the trade name, Lupasol®) or polyoxazolines, which can be prepared according to the procedure of Litt (J. Macromol. Sci. Chem. A9 (5), pp. 703-727 (1975)). Other ABPs can include, but are not limited to, polyacrylamides, polyphosphates, PVPs, PVAs etc. The random asymmetrically branched PEls can be produced primarily through cationic ring opening polymerization of ring-strained cyclic imine monomers, such as, aziridines (ethyleneimine) and azetidines (propyleneimine), with Lewis or Bronsted acids as initiators (Dernier et al., “Ethylenediamine and Other Aziridines,” Academic Press, New York, (1969); and Pell, J. Chem. Soc. 71 (1959)). Since many of the methods are essentially one-pot processes, large quantities of random ABPs can be produced.

The synthetic processes for making ABPs often generate various branch junctures within the macromolecule. In other words, a mixture of terminal and chain branch junctures is distributed throughout the molecular structure. The branching densities of the random ABPs can be lower, and the molecular structure can be more open when compared with dendrimers and dendrigrafts. Although the branch pattern is random, the average ratio of primary, secondary and tertiary amine groups can be relatively consistent with a ratio of about 1:2:1, as described by Dick et al., J. Macromol. Sci. Chem., A4 (6), 1301-1314 (1970) and Lukovkin, Eur. Polym. J. 9, 559 (1973). In one example, the polymer disclosed herein can comprise a ratio of primary, secondary and tertiary amine groups of about 1:2:1.

The presence of the branch junctures can make the random ABPs, such as, asymmetrically branched PEIs, form macromolecules with a possible spherical, ovoid or similar configuration. Within the globular structure, there are various sizes of pockets formed from the imperfect branch junctures at the interior of the macromolecule. Unlike dendrimers and dendrigrafts where interior pockets are always located around the center core of the molecule, the pockets of random ABPs are spread unevenly throughout the entire molecule. As a result, random ABPs possess both exterior and unevenly distributed interior functional groups that can be reacted further with a variety of molecules, thus forming new macromolecular architectures, a modified random ABP of interest.

Although having a core, the functional groups of the regular ABP can also be distributed both at the exterior and in the interior, which is very similar to a random ABP. One such homopolymer is PLL, which can be made as described in U.S. Pat. Nos. 4,289,872; 4,360,646; and 4,410,688. Such homopolymers also can be modified in a manner similar as that for random ABPs, as taught herein, and as known in the art.

In an embodiment of the disclosure, the ABP (for example, either a random asymmetrically branched PEI or a regular asymmetrically branched PLL) is modified with different kinds of primary amine and/or secondary amino groups through, for example, Michael addition or addition of acrylic esters onto amines of the polymer, for example, PEI and PLL homopolymers. The ester groups then can be further derivatized, for example, by an amidation reaction. Thus, for example, such an amidation reaction with, for example, EDA, can yield the addition of an amino group at the terminus of the newly formed branch. Other modifications to the polymer can be made using known chemistries, for example, as provided in the aforementioned “Poly(amines) and Poly(ammonium salts)”. On such addition, a modified ABP, such as, a modified PEI or PLL homopolymer, is formed. As an extension of the ABP, such as, PEI and PLL, the resulting modified ABP also is branched, asymmetrically. Depending on the solvent environment (i.e., pH or polarity), the surface functional groups can carry different charge and charge density. The molecular shape and functional group locations (i.e., functional group back folding) then can be further tuned, based on those characteristic properties.

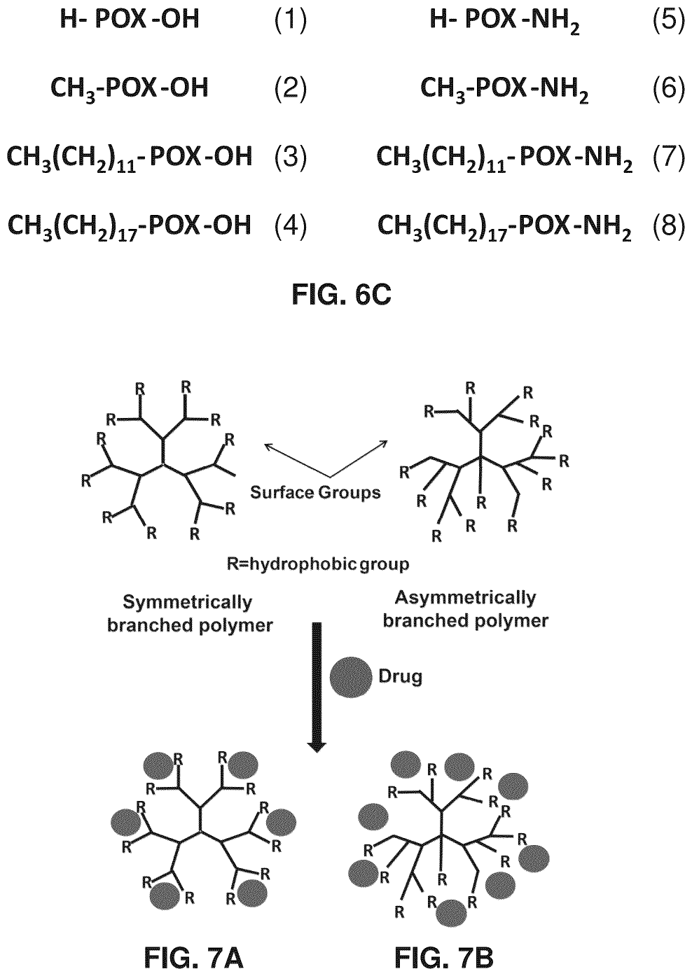

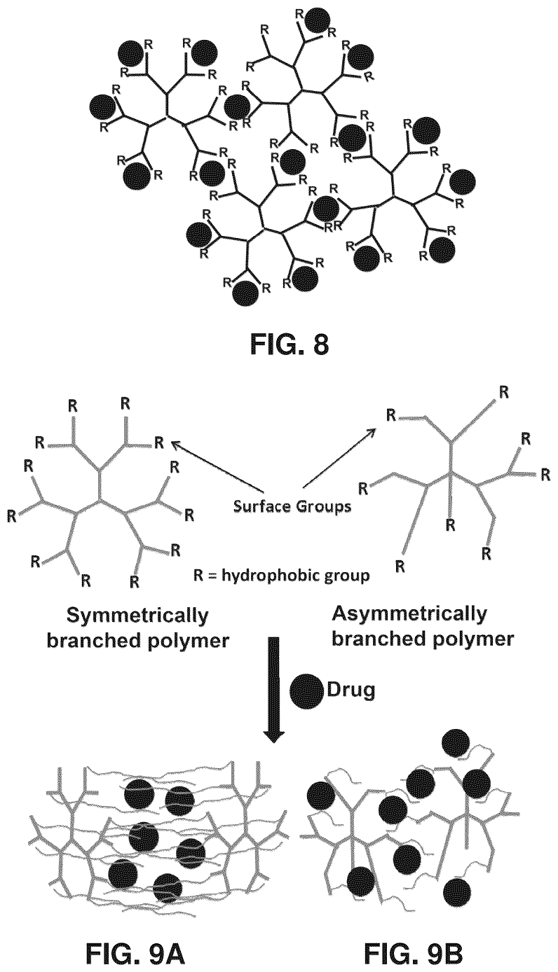

In another embodiment, the modified ABPs can be produced using any of a variety of synthetic schemes that, for example, are known to be amenable to reaction with a suitable site on the homopolymer. Moreover, any of a variety of reagents can be used in a synthetic scheme of choice to yield any of a variety of modifications or additions to the polymer backbone. Thus, for example, in the case of a Michael addition reaction to an amine described above, addition of any of a variety of substituents can be used at the alkylation stage, as provided hereinabove, for example, with an acrylate, which can comprise a saturated or unsaturated hydrocarbon, such as, one comprising one carbon to about 22 carbons, which may be aliphatic, branched, saturated, aromatic, ringed or combination thereof. The hydrocarbon can have 2 to 22 carbons in one example, 4 to 22 carbons in another example, 6 to 22 carbons in yet another example, 7 to 22 carbons in yet another example, 8 to 22 carbons in yet another example, 10 to 22 carbons in yet another example, 12 to 22 carbons in yet another example, 14 to 22 carbons in yet another example, 16 to 22 carbons in yet another example, 18 to 22 carbons in a further example, and 20 to 22 carbons in yet a further example. In one particular example, the first terminal group can comprise 18 carbons, such as, a (CH₃(CH₂)₁₇)-group. Suitable reactants include methyl acrylate, ethyl acrylate, propyl, acrylate, butyl acrylate, pentyl acrylate, hexyl acrylate, heptyl acrylate, octyl acrylate, nonyl acrylate, decyl acrylate, undecyl acrylate, dodecyl acrylate and the like, and mixtures thereof. Similarly, at the amidation stage in the example exemplified above, any of a variety of amines can be used in the methods provided herein and known in the art. For example, EDA, monoethanolamine, tris(hydroxymethyl)aminomethane, alkyl amine, allyl amine or any amino-modified polymers, including PEG, perfluoropolymers, polystyrene, polyethylene, polydimethylsiloxane, polyacrylate, polymethylmethacrylate and the like, and mixtures thereof, can be used. In addition, linking of the hydrophobic groups, including aliphatic (e.g., hydrocarbons from C₁ to about C₂₂) groups, aromatic groups, polyethylene polymers, polystyrene polymers, perfluoropolymers, polydimethylsiloxanes, polyacrylates, polymethylmethacrylates, as well as, hydrophilic groups, including an OH group, hydrophilic polymers, such as, PEOX, PEG, PEO etc., to a modified ABP can be achieved by using, for example, epoxy reactions, amidation reactions, Michael addition reactions, including using an —SH or an —NH₂ group reacted with maleimide, aldehyde/ketone-amine/hydrazide coupling reactions, iodo/iodoacetyl-SH coupling reactions, hydroxylamine-aldehyde/ketone coupling reactions etc. Such synthetic strategies allow not only asymmetric growth of the molecule, where more pockets are introduced, but also addition of multiple functional groups at both the interior and the exterior of the structure. The homopolymer can be modified further using the same or a different synthetic process until the desired ABPs with appropriate molecular weight and functional groups are attained. In addition, hydrophobic and hydrophilic properties, as well as charge density of such homopolymers, can be tailored to fit specific application needs using appropriate monomers for constructing the homopolymer and suitable modification reactions. An example of a modified ABP is shown in FIG. 5. A modified hyperbranched PEI is shown in FIG. 6A.

In another embodiment of the disclosure, a focal point (merged from various reactive chain ends during a convergent synthesis) of a random ABP, such as, POX, can be terminated or reacted with another small molecule to generate various functional groups at the homopolymeric chain end, including primary, secondary or tertiary amines, carboxylate, hydroxyl, alkyl, fluoroalkyl, aryl, PEG, acetate, amide and/or ester groups. Alternatively, various initiators also can be utilized so that the same type of functional group can be introduced at the surface groups where a polymerization begins during a convergent synthesis (J. Macromol. Sci. Chem. A9 (5), pp. 703-727 (1975)),

An alkyl surface-modified, randomly branched poly(2-ethyloxazoline) with a primary amine group at the focal point of the branched polymer can be prepared using the Litt and Warakomski procedures, supra. For example, CH₃(CH₂)₁₇—Br can be utilized as an initiator for 2-ethyloxazoline polymerization through a cationic ring opening process to generate a randomly branched polymer, followed by quenching with N-tert-butyloxycarbonylpiperazine (N-Boc-piperazine) or EDA. Termination with a large excess of EDA allows the hydrophobically modified branched poly(2-ethyloxazoline) polymer to be functionalized with a primary amine group at the focal point (FIG. 6B). Alternatively, N-Boc-piperazine-terminated hydrophobically-modified branched poly(2-ethyloxazoline) polymer also can be deprotected to generate a free amino group at the focal point. In some cases, the polymer can comprise a modified branched poly(2-ethyloxazoline) functionalized with primary, secondary or tertiary amines, carboxylate, hydroxyl, alkyl, fluoroalkyl, aryl, PEG, acetate, amide or ester groups at a focal point of the polymer where two or more reactive chain ends merged during a convergent synthesis.

In some cases, an alkyl surface-modified, randomly branched poly(2-ethyloxazoline) can have a hydroxyl group at the focal point of the branched polymer. For example, the focal point of the polymer can be hydrolyzed to, for example, a hydroxyl group on dissolving in water (e.g., containing, for example, 1N Na₂CO₃).

In some cases, the focal point of the polymer mentioned herein can comprise a second terminal group modified with a hydrophilic moiety.

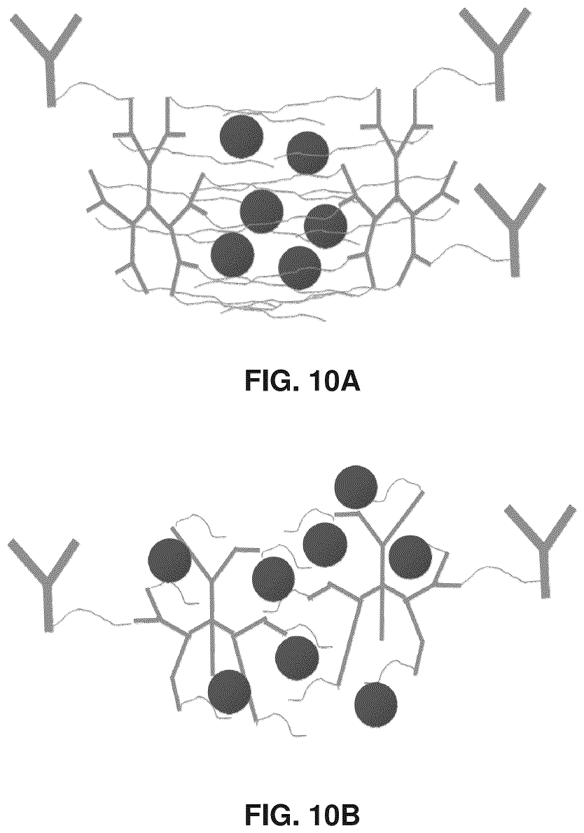

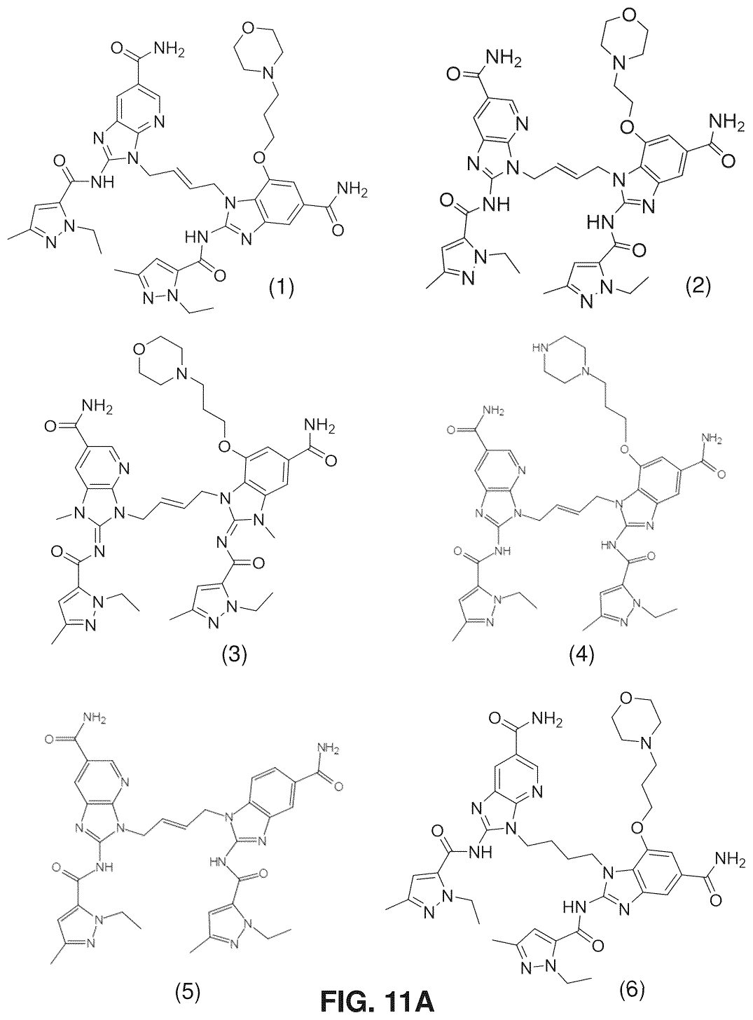

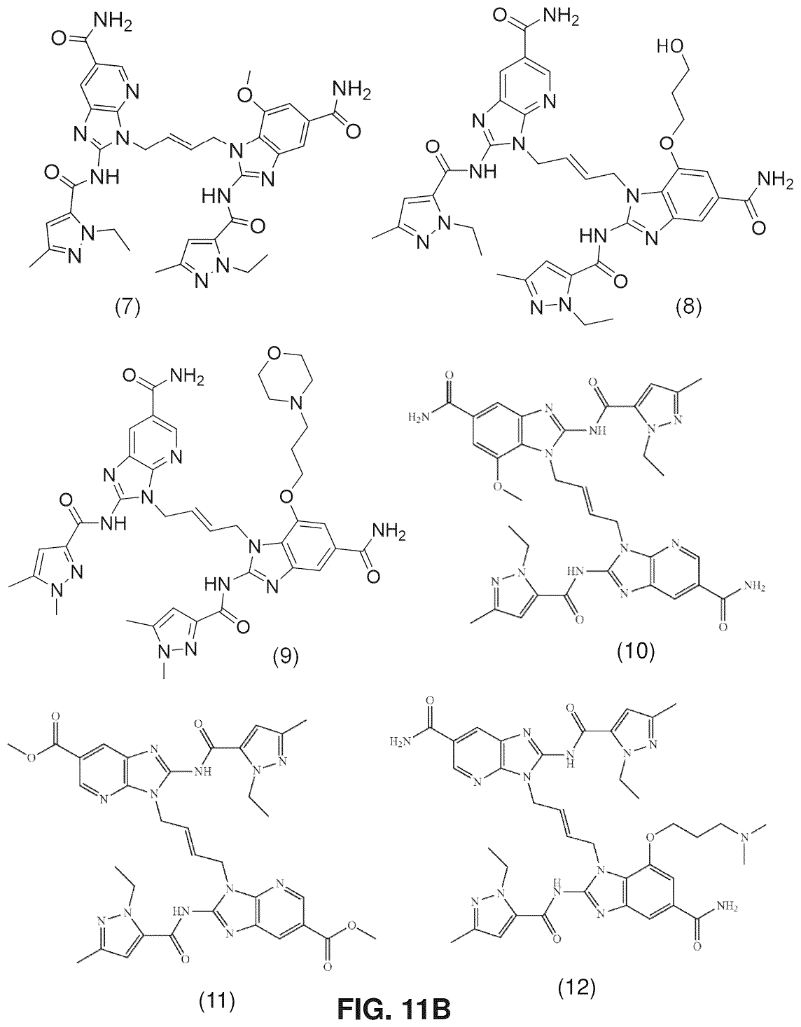

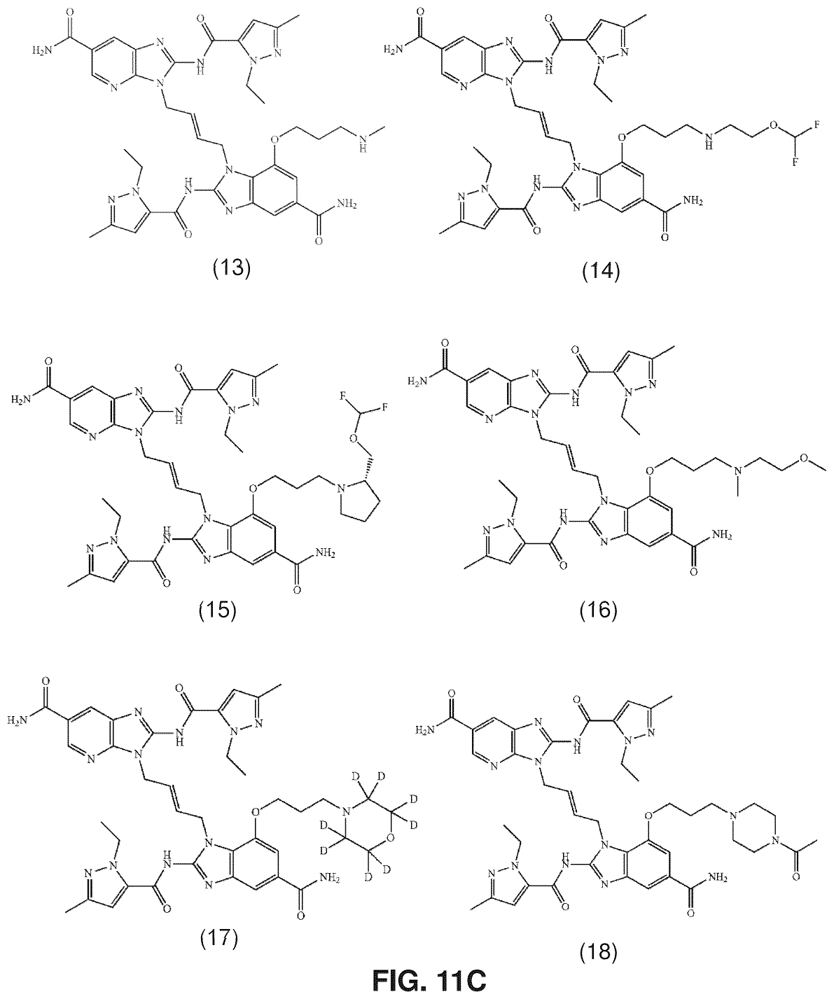

While introduction of a primary amine group to a hydrophobically-modified branched poly(2-oxazoline) homopolymer enhances drug solubility and produces bioactive agent-induced nanoaggregates (such as shown in FIG. 7A-FIG. 7B, FIG. 8, FIG. 9A-FIG. 9B), the primary amine group also allows attachment of various targeting groups, such as, an antibody, antigen-binding portion thereof, an antigen or a member of a binding pair, such as, to the hydrophobically modified branched poly(2-oxazoline) polymer (FIG. 10A-FIG. 10B). This can be particularly useful prior to mixing the polymer and a bioactive agent. Such nanoaggregates or nanoparticles containing such targeting groups and modifications thereto can provide targeting ability on the nanoaggregate with a bioactive agent, and enable the bioactive agent to be released preferentially or solely at desired treatment locations. As mentioned above, it is preferred to have a polymer that in a range of from 1% to 100% of the second terminal group is free from primary amine when the polymer is used to mix with a bioactive agent to produce the pharmaceutical composition of this disclosure.

As disclosed herein, modified branched polymers (MBP), such as, a hydrophobically-modified homopolymer, including SBPs, ABPs, or a combination thereof, can be used to generate an encapsulating polymer or nanocapsule for solubilizing a water insoluble bioactive agent. In an organic solvent environment, the hydrophilic or amphiphilic interior can be poly(2-oxazoline), poly(2-substituted oxazolines), wherein the poly(2-substituted oxazoline) can comprise poly(2-methyloxazoline), poly(2-ethyloxazoline), poly(2-propyloxazoline), poly(isopropyloxazoline) (PiPOX), or a combination thereof, PEG, PEO, polyphosphonate and the like. The hydrophobic exterior can comprise aliphatic hydrocarbons (such as, from C₁ to about C₂₂), aromatic hydrocarbons, polyethylene polymers, polystyrene polymers, perfluoropolymers, polydimethylsiloxanes, polyacrylates, polymethylmethacrylates and the like. In an aqueous environment, the reverse is true. In the drug-induced nanoaggregates in an aqueous environment, the drug molecules, such as, water insoluble bioactive agent can be associated with the hydrophobic groups/domains of the MBP's (FIG. 9A-FIG. 9B). The branching density (e.g., from low generation, such as, star and comb homopolymers, to high generation of dendrimers and dendrigrafts), as well as the amount of hydrophobic surface group coverage (e.g., from 0% to 100% coverage) of the branched homopolymers can affect homopolymer solubility, which in turn, also can affect ability to dissolve or to adsorb/absorb a bioactive agent. For example, increase in branching density and amount of hydrophobic group coverage will make a homopolymer more compatible with a bioactive agent.

In further examples, the ABPs and SBPs with from about 0.1 to about 30% or more surface hydrophobic component by weight are effective at solubilizing or dispersing poorly water soluble or water insoluble bioactive agent. In addition, branched homopolymers utilizing, for example, a POX, a PMOX, a PEOX, a PPOX, PEO/PEG, polyacrylamides, polyphosphates, PVPs and PVAs are soluble in both water and in various organic solvents, thereby facilitating forming bioactive agent-containing nanoparticles or nanoaggregates. The good water solubility along with good hydrophobic drug miscibility in an aqueous solution, with or without other organic solvents, makes such homopolymers useful for enhancing solubility of poorly water soluble bioactive agents. For example, the homopolymers of interest simplify manufacturing processes and decrease production cost by reducing formulation steps, processing time, as well as the need to use complex and expensive equipment currently used in the pharmaceutical industry. If additional branching densities are needed, the SBPs or ABPs first can be modified with additional groups as described herein, and then, for example, attached with additional hydrophobic functional groups for enhancing solubility of a bioactive agent.

In one example, a polymer is configured to have effective branching density, amount of hydrophobic groups at the surface of the polymer, or a combination thereof, for encapsulating a bioactive agent that is in water insoluble form in the nanoaggregate. The effective branching density, the amount of hydrophobic groups at the surface of the polymer, or a combination thereof, can be modified as described above and hereafter.

In one example, a polymer can have hydrophobic groups, including aliphatic (e.g., hydrocarbons from C₁ to about C₂₂) groups, aromatic groups, polyethylene polymers, polystyrene polymers, perfluoropolymers, polydimethylsiloxanes, polyacrylates, polymethylmethacrylates, linked to a POX polymer including a PEOX polymer and further modified by EDA. The POX polymer can be a homopolymer polymerized from a repeating unit comprising a single monomer or a repeating unit comprising two or more monomers in each repeating unit.

In some cases, a polymer can comprise asymmetrically branched polymers (ABP) or dendritic asymmetrically branched polymer, such as, asymmetrically branched PEOX formed from the initiators and monomers at ratios disclosed herein. In some cases, a polymer can comprise randomly branched poly(2-ethyloxazoline) having one or more first terminal groups, such as, a hydrophobic moiety disclosed herein, and one second terminal group positioned at the focal point of the branched polymer, such as, the modified randomly branched PEOX formed by polymerizing reactive linear PEOX polymers with chain transfer polymerization convergent synthesis as illustrated in FIG. 6B.

In some cases, polymers can have different first terminal groups and different second terminal groups. Some examples are shown in FIG. 6C: Polymer (1)-Polymer (4) having-OH as the second terminal group and Polymer (5)-Polymer (8) having-NH₂ as the second terminal group, Polymer (1) and Polymer (5) having H as the first terminal group, Polymer (2) and Polymer (6) having-CH₃ as the first terminal group, Polymer (3) and Polymer (7) having C₁₂ as the first terminal group, and Polymer (4) and Polymer (8) having C₁₈ as the first terminal group. The polyoxazoline (POX) polymer can be a linear polymer, a branched polymer, or a polymer having a combination of one or more linear portions and one or more branched portions. The polyoxazoline (POX) can comprise poly(2-methyloxazoline), poly(2-ethyloxazoline), poly(2-propyloxazoline), poly(isopropyloxazoline) (PiPOX), or a combination thereof. Although specific first terminal groups and second terminal groups are described above, other first and second groups disclosed herein can be suitable. In some cases, the second terminal group can comprise a group modified by an amine, amide, imine, imide, carboxyl, hydroxyl, ester, ether, acetate, phosphate, ketone, aldehyde, sulfonate, or a combination thereof. The first terminal group and the second terminal group can be modified according to methods and processes known to those skilled in the art. If needed, one or more reagents, linkers or intermediates known to those skilled in the art can be used.

In any of pharmaceutical compositions disclosed above and hereafter, the polyoxazoline can comprise a molar ratio of monomer to initiator in a range of from 50:1 to 80:1.

The pharmaceutical composition can comprise additional polymers selected from ABPs, ABPs, MBPs, such as, symmetrically branched PAMAM or PPI dendrimers, polyether dendrimers, polyester dendrimers, comb-branched/star-branched polymers, such as, those containing PEO, PEG, PMOX or PEOX, polystyrene, and comb-branched dendrigrafts, such as, those containing PEOX, PMOX, PEI, polylysine (e.g., branched PLL), polyacrylamides, polyphosphates, PVPs, PVAs or a combination thereof. The random asymmetrically branched PEIs can be produced primarily through cationic ring opening polymerization of ring-strained cyclic imine monomers, such as, aziridines (ethyleneimine) and azetidines (propyleneimine), or a combination thereof. The additional polymers can be mixed with the nanoaggregate disclosed herein. In one example, one or more additional polymers can be mixed with a nanoaggregate after the nanoaggregate has formed.

Suitable to the pharmaceutical composition, process, method and use disclosed herein throughout this disclosure, the term “bioactive agent” refers to a substance that can be a natural or synthetic small molecule-based drug, inorganic-based drug, biological drug, natural or synthetic large molecule-based drug, modifications and/or derivatives thereof, or a combination thereof, as disclosed herein. The bioactive agent can include a natural or synthetic small molecule-based drug, inorganic-based drug, biological drug, natural or synthetic large molecule-based drug, modifications and/or derivatives thereof, or a combination thereof, wherein at least one drug is poorly water soluble or water insoluble. A drug of interest can be a small molecule, a salt thereof in which the molecule is modified to be water insoluble or poorly water soluble or can be a biological molecule which is modified to be water insoluble or poorly water soluble, particularly when a drug has improved properties, such as, improved bioavailability, less toxicity, better pharmacokinetics, or a combination thereof, in a water insoluble or poorly water soluble form. Suitable examples can include drugs which are poorly water soluble or water insoluble or can be modified to be water insoluble or poorly water soluble for an improved property. A bioactive agent can include growth agents; AIDS adjunct agents; alcohol abuse preparations, such as, agents for treating dependence or withdrawal; Alzheimer's Disease treatment agents; Amyotrophic Lateral Sclerosis treatment agents; analgesics; anesthetics; anticonvulsants; antidiabetic agents; antidotes; antifibrosis therapy agents; antihistamines; anti-infective agents, such as, antibiotics, antivirals, antifungals, amebicides, antihelmintics, antimalarials, leprostatics and so on; antineoplastic agents; antiparkinsonian agents; antirheumatic agents; appetite stimulants; biological response modifiers; biologicals; blood modifiers, such as, anticoagulants, colony stimulating factors, hemostatics, plasma extenders, thrombin inhibitors and so on; bone metabolism regulators; cardioprotective agents; cardiovascular agents, such as, adrenergic blockers, adrenergic stimulators, angiotensin converting enzyme (ACE) inhibitors, antiarrhythmics, antilipemic agents, calcium channel blockers, diuretics, vasopressors and so on; central nervous system (CNS) stimulants; cholinesterase inhibitors; contraceptives; fertility treatment agents; ovulation stimulators; cystic fibrosis managements agents; detoxifying agents; diagnostics; dietary supplements; dopamine receptor agonists; endometriosis management agents; enzymes; erectile dysfunction treatment agents; foot care products; gastrointestinal (GI) treatment agents, such as, antacids, antidiarrheals, antiemetics, antiflatulants, bowel evacuants, digestive enzymes, histamine receptor agonists, laxatives, proton pump inhibitors, prostaglandins and so on; Gaucher's Disease treatment agents; gout treatment agents; homeopathic remedies; skin treatments; vitamins; nutrients; hormones; hypercalcemia management treatment agents; hypocalcemia management treatment agents; immunomodulators; immunosuppressants; levocarnitine deficiency treatment agents; mast cell stabilizers; migraine treatment agents, motion sickness treatment products, such as, Benadryl and Phenergan; decongestants; antihistamines; cough suppressants; multiple sclerosis treatment agents; muscle relaxants; nasal treatment agents, such as, anti-inflammatoires; smoking cessation aids; appetite suppressants; nucleoside analogs; obesity management agents; ophthalmic preparations, such as, antibiotics, antiglaucoma agents, artificial tears, lubricants and so on; sexual aids; osteoporosis treatment agents; otic preparations, such as, antiinfectives and cerumenolytics; minerals; oxytocics; parasympatholytics; parasympathomimetics; patent ductus arteriosus agents; phosphate binders; porphyria agents; prostaglandins; psychotherapeutic agents; radiopaque agents; respiratory agents, such as, antiinflammatories, antitussives, bronchodilators, decongestants, expectorants, leukotrienes antagonists, surfactants and so on; salt substitutes; sedatives; hypnotics; skin and mucous membrane treatment agents, such as, acne treatments; anorectal treatment agents, such as, hemorrhoid treatments and enemas; antiperspirants; antipruritics; antipsoriatic agents; antiseborrheic agents; burn treatment agents; cleansing agents; depigmenting agents; emollients; hair growth retardants; hair growth stimulators; keratolytics; hair problem treatment agents; mouth and throat problem treatment agents; photosensitizing agents; wart treatment agent; wound care treatment agents, or a combination thereof. A bioactive agent can also include over the counter pharmaceutics and products, such as, deodorants; Tourette's Syndrome agents; tremor treatments; urinary tract agents, such as, acidifiers, alkalinizers; antispasmodics; benign prostatic hyperplasia treatment agents; calcium oxalate stone preventors; enuresis management agents; vaginal preparations, such as, antiinfectives, hormones and so on; vasodilators; vertigo treatment agents, Wilson's Disease treatments and so on.

Further examples of bioactive agent can include forms of drugs which may be modified, for example, as salts, ionized or hydrophilic forms that can be modified to remove such functional groups, modifications and the like to yield non-modified or other forms of bioactive agents which are poorly water soluble or water insoluble. If two or more bioactive agents are comprised in the pharmaceutical composition, at least one of the bioactive agents can be or has been modified to be water insoluble or poorly water soluble. Examples of such bioactive agents can include, analgesics/antipyretics (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate and the like); anesthetics (e.g., cyclopropane, enflurane, halothane, isoflurane, methoxyflurane, nitrous oxide, propofol and the like); antiasthmatics (e.g., azelastine, ketotifen, traxanox, amlexanox, cromolyn, ibudilast, montelukast, nedocromil, oxatomide, pranlukast, seratrodast, suplatast tosylate, tiaramide, zafirlukast, zileuton, beclomethasone, budesonide, dexamethasone, flunisolide, triamcinolone acetonide and the like); antibiotics (e.g., neomycin, streptomycin, chloramphenicol, cephalosporin, ampicillin, penicillin, tetracycline and the like); quinolone, fluoroquinolone, antidepressants (e.g., nefopam, oxypertine, doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, trimipramine maleate, protriptyline hydrochloride and the like); antidiabetics (e.g., biguanides, hormones, sulfonylurea derivatives, and the like); antifungal agents (e.g., griseofulvin, ketoconazole, amphotericin B, nystatin, candicidin and the like); antihypertensive agents (e.g., propanolol, propafenone, oxyprenolol, nifedipine, reserpine, trimethaphan camsylate, phenoxybenzamine hydrochloride, pargyline hydrochloride, deserpidine, diazoxide, guanethidine monosulfate, minoxidil, rescinnamine, sodium nitroprusside, rauwolfia serpentina, alseroxylon, phentolamine mesylate, reserpine and the like); anti-inflammatoires (e.g., non-steroidal compounds, such as, indomethacin, naproxen, ibuprofen, ramifenazone, piroxicam and so on, and steroidal compounds, such as, cortisone, dexamethasone, fluazacort, hydrocortisone, prednisolone, prednisone and the like); antineoplastics (e.g., adriamycin, cyclophosphamide, actinomycin, bleomycin, daunorubicin, doxorubicin, epirubicin, gemcitabine, mitomycin, methotrexate, fluorouracil, carboplatin, nitrogen mustard β-chloronitrosourea (carmustine, BCNU), -chloroethyl-3-cyclohexyl-1-nitrosourea (lomustine, CCNU), methyl-CCNU, cisplatin, etoposide, interferons, camptothecin and derivatives thereof, phenesterine, Taxol and derivatives thereof, taxotere and derivatives thereof, vinblastine, vincristine, tamoxifen, etoposide, piposulfan and the like); antianxiety agents (e.g., lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, dantrolene and the like); immunosuppressive agents (e.g., cyclosporine, azathioprine, mizoribine, FK506 (tacrolimus), rapamycin (sirolimus) and the like); antimigraine agents (e.g., ergotamine tartrate, propanolol hydrochloride, isometheptene mucate, dichloralphenazone and the like); sedatives/hypnotics (e.g., barbiturates (e.g., pentobarbital, pentobarbital sodium, secobarbital sodium and the like) or benzodiazepines (e.g., flurazepam hydrochloride, triazolam, tomazeparm, midazolam hydrochloride and the like); antianginal agents (e.g., beta-adrenergic blockers, calcium channel blockers (e.g., nifedipine, diltiazem hydrochloride and the like) and nitrates (e.g., nitroglycerin, isosorbide dinitrate, pentaerythritol tetranitrate, erythrityl tetranitrate and the like)); antipsychotic agents (e.g., haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, prochlorperazine and the like); antimanic agents (e.g., lithium carbonate); antiarrhythmics (e.g., bretylium tosylate, esmolol hydrochloride, verapamil hydrochloride, amiodarone, encamide hydrochloride, digoxin, digitoxin, mexiletine hydrochloride, disopyramide phosphate, procainamide hydrochloride, quinidine sulfate, quinidine gluconate, quinidine polygalacturonate, flecamide acetate, tocamide hydrochloride, lidocaine hydrochloride and the like); antiarthritic agents (e.g., phenylbutazone, sulindac, penicillamine, salsalate, piroxicam, azathioprine, indomethacin, meclofenamate sodium, gold sodium thiomalate, ketoprofen, auranofin, aurothioglucose, tolmetin sodium and the like); antigout agents (e.g., colchicine, allopurinol and the like); anticoagulants (e.g., heparin, heparin sodium, warfarin sodium and the like); thrombolytic agents (e.g., urokinase, streptokinase, altoplase and the like); antifibrinolytic agents (e.g., aminocaproic acid); hemorheologic agents (e.g., pentoxifylline); antiplatelet agents (e.g., aspirin, empirin, ascriptin and the like); anticonvulsants (e.g., valproic acid, divalproate sodium, phenyloin, phenyloin sodium, clonazepam, primidone, phenobarbital, phenobarbital sodium, carbamazepine, amobarbital sodium, methsuximide, metharbital, mephobarbital, mephenyloin, phensuximide, paramethadione, ethotoin, phenacemide, secobarbitol sodium, clorazepate dipotassium, trimethadione and the like); antiparkinson agents (e.g., ethosuximide and the like); antihistamines/antipruritics (e.g., hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, trimprazine tartrate and the like); agents useful for calcium regulation (e.g., calcitonin, parathyroid hormone and the like); antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, colistin sulfate and the like); antiviral agents (e.g., interferon γ, zidovudine, amantadine hydrochloride, ribavirin, acyclovir and the like); antimicrobials (e.g., cephalosporins (e.g., cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium and the like)), penicillins (e.g., ampicillin, amoxicillin, penicillin G benzathine, cyclacillin, ampicillin sodium, penicillin G K, penicillin V K, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, nafcillin sodium and the like), erythromycins (e.g., erythromycin ethylsuccinate, erythromycin, erythromycin estolate, erythromycin lactobionate, erythromycin stearate, erythromycin ethylsuccinate and the like), tetracyclines (e.g., tetracycline hydrochloride, doxycycline hyclate, minocycline hydrochloride and the like), and the like); anti-infectives (e.g., granulocyte-macrophage colony stimulating factor, GM-CSF); bronchodilators (e.g., sympathomimetics (e.g., epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate)); anticholinergic agents (e.g., ipratropium bromide); xanthines (e.g., aminophylline, dyphylline, metaproterenol sulfate, aminophylline); mast cell stabilizers (e.g., cromolyn sodium); inhalant corticosteroids (e.g., flunisolide, beclomethasone dipropionate monohydrate and the like), salbutamol, beclomethasone dipropionate (BDP), ipratropium bromide, budesonide, ketotifen, salmeterol, xinafoate, terbutaline sulfate, triamcinolone, theophylline, nedocromil sodium, metaproterenol sulfate, albuterol, flunisolide and the like); hormones (e.g., androgens (e.g., danazol, testosterone cypionate, fluoxymesterone, ethyltostosterone, testosterone enanthate, methyltestosterone, fluoxymesterone, testosterone cypionate and the like); estrogens (e.g., estradiol, estropipate, conjugated estrogens and the like), progestins (e.g., methoxyprogesterone acetate, norethindrone acetate and the like)), corticosteroids (e.g., triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate and the like), thyroid hormones (e.g., levothyroxine sodium); and the like; hypoglycemic agents (e.g., human insulin, purified beef insulin, purified pork insulin, glyburide, chlorpropamide, glipizide, tolbutamide, tolazamide and the like); hypolipidemic agents (e.g., clofibrate, dextrothyroxine sodium, probucol, lovastatin, niacin and the like); proteins (e.g., DNase, alginase, superoxide dismutase, lipase and the like); nucleic acids (e.g., sense or anti-sense nucleic acids encoding any therapeutically useful protein, including any of the proteins described herein, and the like); agents useful for erythropoiesis (e.g., erythropoietin); antiulcer or antireflux agents (e.g., famotidine, cimetidine, ranitidine hydrochloride and the like); antinauseants or antiemetics (e.g., meclizine hydrochloride, nabilone, prochlorperazine, dimenhydrinate, promethazine hydrochloride, thiethylperazine, scopolamine and the like); oil-soluble vitamins (e.g., vitamins A, D, E, K and the like); mitotane, visadine, halonitrosoureas, anthrocyclines, ellipticine and the like; STING inhibitors such as, C-176, C-170 and C-171; STING activators, such as 3′,3′-cGAMP (3′,3′-cyclic GMP-AMP, cyclic GMP-AMP, cGAMP); STING agonists, such as, SR-717 lithium, α-mangostin or diABZI (amido benzimidazole) STING agonist (diABZI STING agonist-1, Compound 3), STING agonist-1 (G10), CF501 (Formula (1)), CF502 (Formula (5)), CF504 (Formula (7)), CF505 (Formula (8)), CF508 (Formula (4)), CF509 (Formula (6)), CF510 (Formula (2)), CF511 (Formula (9)) (Liu, et al., Cell Research, 1-19, 2022. https://doi.org/10.1038/s41422-022-00612-2), or MSA-2 (Tocris, Minneapolis, MN); STING antagonist, such as, SN-011 (GUN35901) or H-151; indoleamine dioxygenase (IDO) inhibitors or IDO1 inhibitors, such as, Epacadostat (INCB24360), BMS-986205, PF-0684003, Navoximod, Indoximod, NLG802 (Indoximod prodrug) or LY3381916; and a combination thereof.

In some cases, the bioactive agent can comprise any one of the bioactive agents listed above and hereafter. In some cases, the bioactive agent can comprise two or more of the bioactive agents listed above and hereafter.

In some cases, the bioactive agent can comprise immunoglobin, such as, IgG, IgM, one or more molecules disclosed and prepared according to processes and method described in U.S. Pat. No. 10,688,048, hereby incorporated by reference.

In some cases, the bioactive agent can comprise at least one STING polypeptide or a part thereof, a nucleic acid encoding the STING polypeptide or a part thereof, a STING inhibitor, a STING activator, a STING agonist, a STING antagonist, a STING modulating molecule, an IDO inhibitor, an IDO1 inhibitor, or a combination thereof. The bioactive agent can comprise RNA, mRNA, siRNA, sgRNA, DNA, oligo, or a combination thereof, that each encodes one of the aforementioned STING polypeptide or a part thereof, a STING inhibitor, a STING activator, a STING agonist, a STING antagonist, a STING modulating molecule, or the IDO inhibitor or IDO1 inhibitor.

In some cases, the bioactive agent can comprise a STING (stimulator of interferon genes) protein, STING agonists, STING activators, STING inhibitors, STING antagonists or a combination thereof. In some cases, the bioactive agent can comprise one or more IDO or IDO1 inhibitors. Any of the STING protein, STING agonists, STING activators, STING inhibitors, STING antagonists, IDO inhibitors or IDO1 inhibitors, disclosed herein or discovered thereafter can be suitable. In some cases, the bioactive agent can comprise STING modulating molecules, such as benzimidazole compounds disclosed by Liu, et al. (Cell Research, 1-19, 2022), in patent publications WO2017175156A1 and WO2020156363, pyridinyl imidazole compounds disclosed in patent publications WO2019134705, WO2020010451 and US20200031825, or a combination.

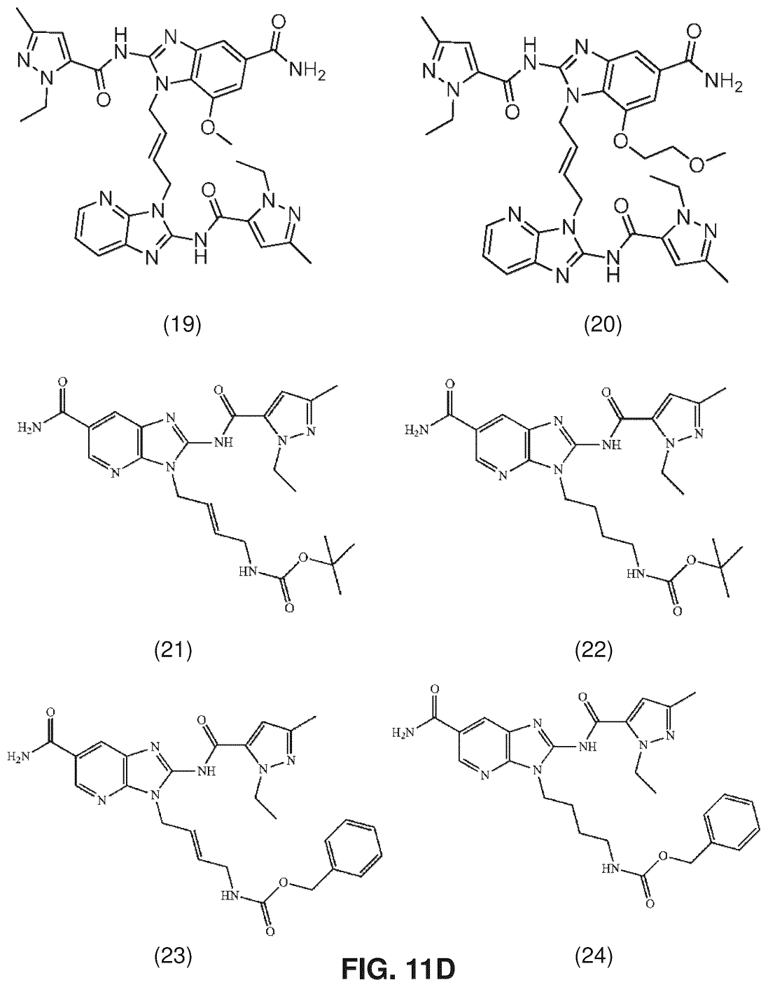

In some cases, the bioactive agent can comprise one or more STING agonists. In some cases, the STING agonist can comprise one or more compounds having the formula (1)-(29) (FIG. 11A-FIG. 11E),

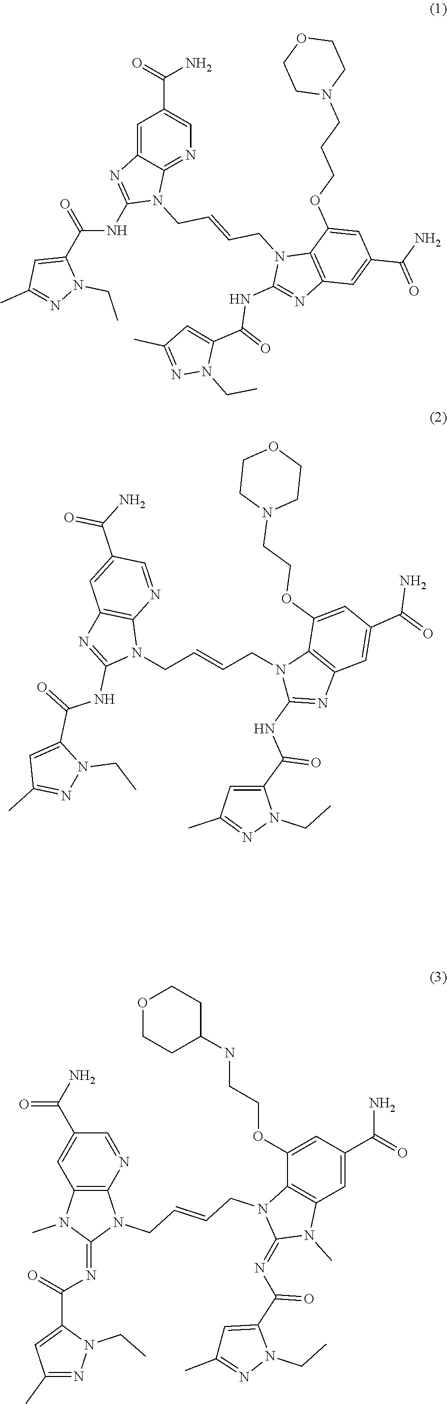 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CCOCC3)c21 ; CCn1nc(C)cc1C(=O)/N=c1\n(C)c2cc(C(N)=O)cnc2n1C/C=C/Cn1/c(=N/C(=O)c2cc(C)nn2CC)n(C)c2cc(C(N)=O)cc(OCCNC3CCOCC3)c21]

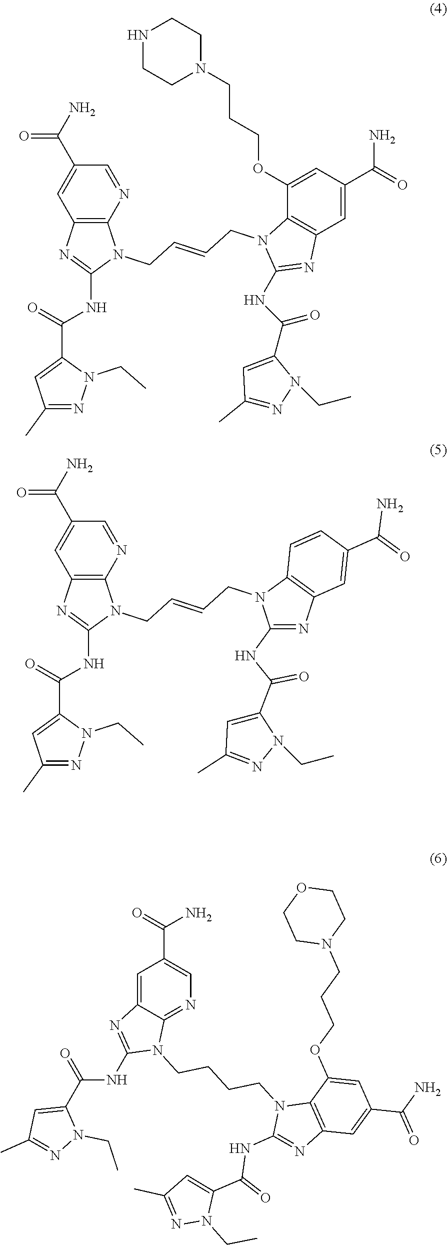 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cnc21 ; CCn1nc(C)cc1C(=O)Nc1nc2cc(C(N)=O)cnc2n1CCCCn1c(NC(=O)c2cc(C)nn2CC)nc2cc(C(N)=O)cc(OCCCN3CCOCC3)c21]

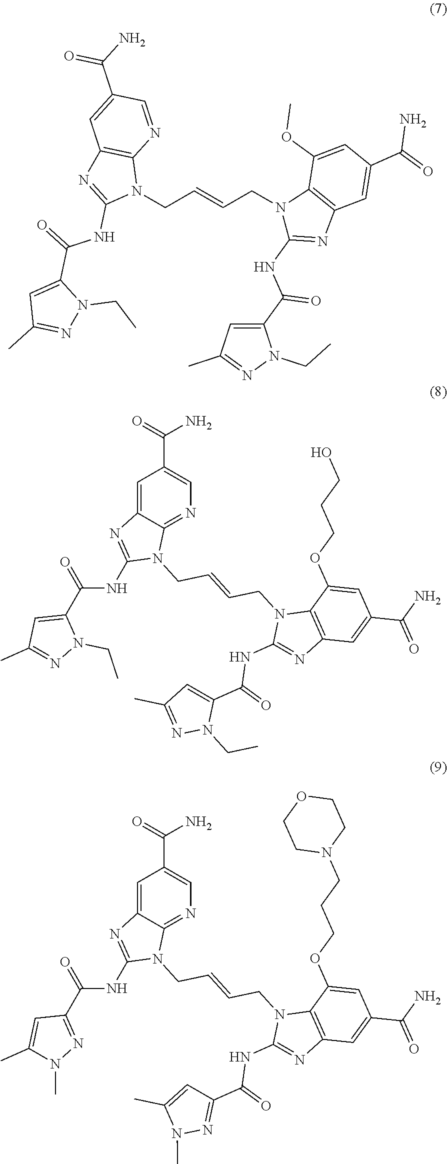 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OC)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCO)c21 ; Cc1cc(C(=O)Nc2nc3cc(C(N)=O)cnc3n2C/C=C/Cn2c(NC(=O)c3cc(C)n(C)n3)nc3cc(C(N)=O)cc(OCCCN4CCOCC4)c32)nn1C]

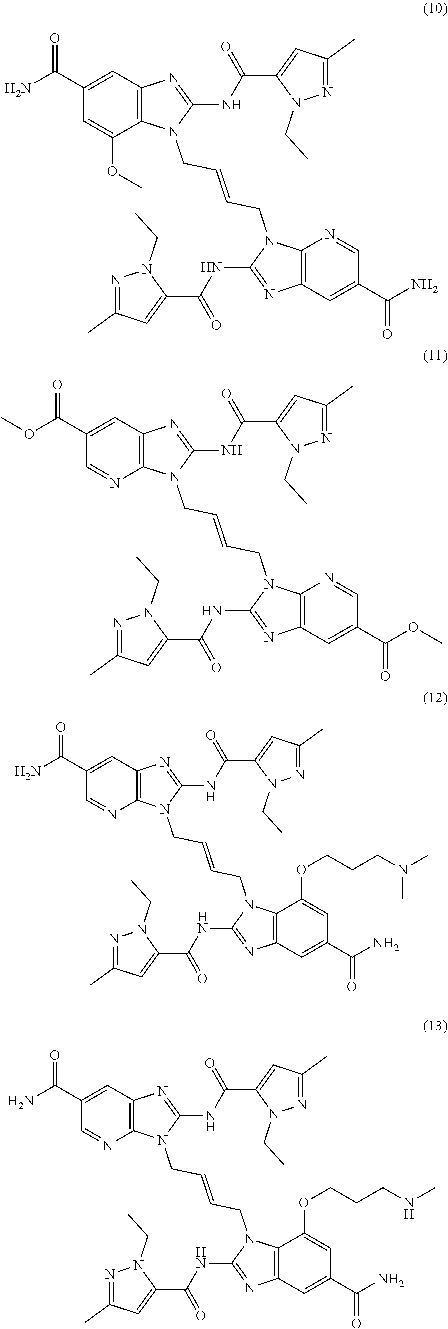

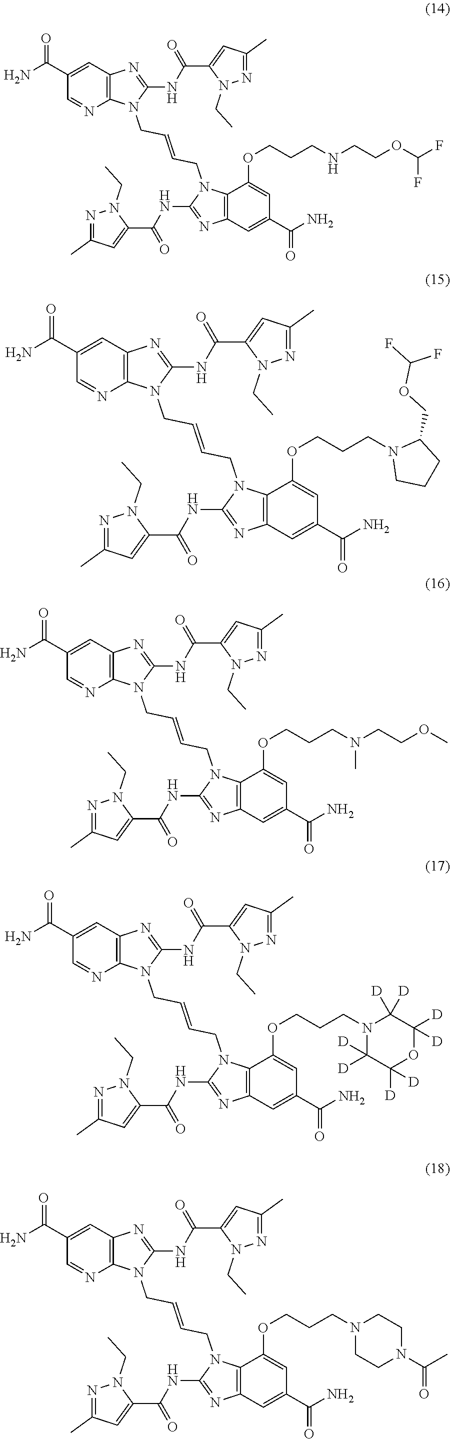

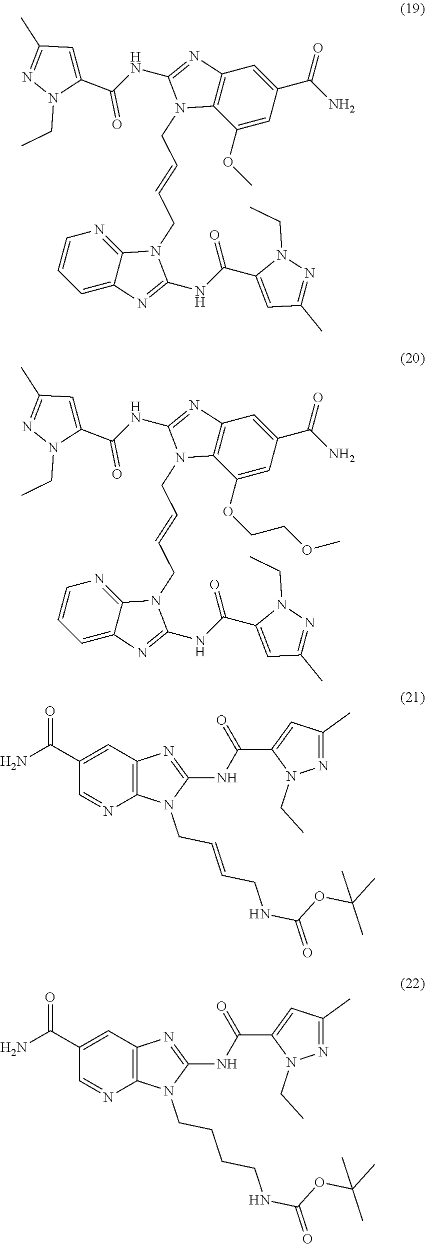

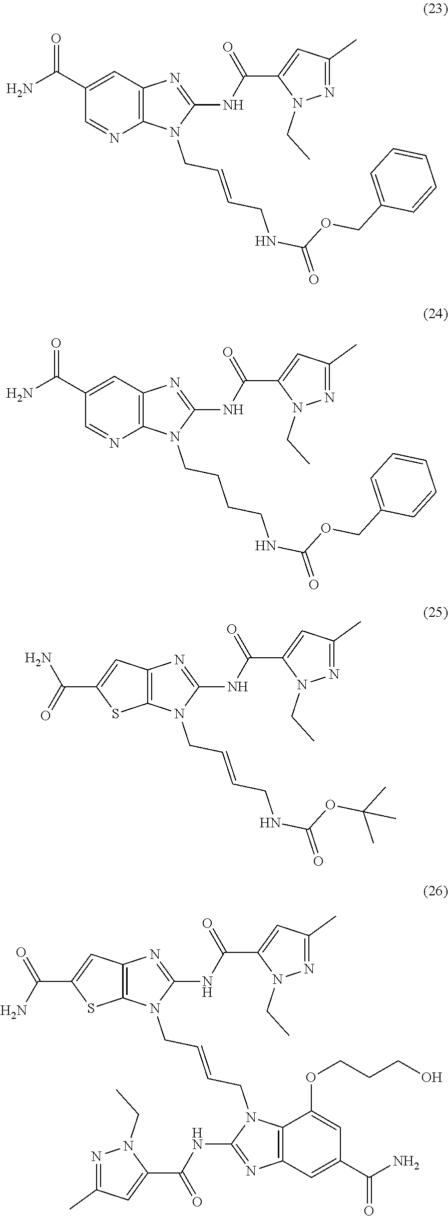 [SMILES: CCn1nc(C)cc1C(=O)Nc1nc2cc(C(N)=O)cnc2n1C/C=C/CNC(=O)OCc1ccccc1 ; CCn1nc(C)cc1C(=O)Nc1nc2cc(C(N)=O)cnc2n1CCCCNC(=O)OCc1ccccc1 ; CCn1nc(C)cc1C(=O)Nc1nc2cc(C(N)=O)sc2n1C/C=C/CNC(=O)OC(C)(C)C ; CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCO)c21]

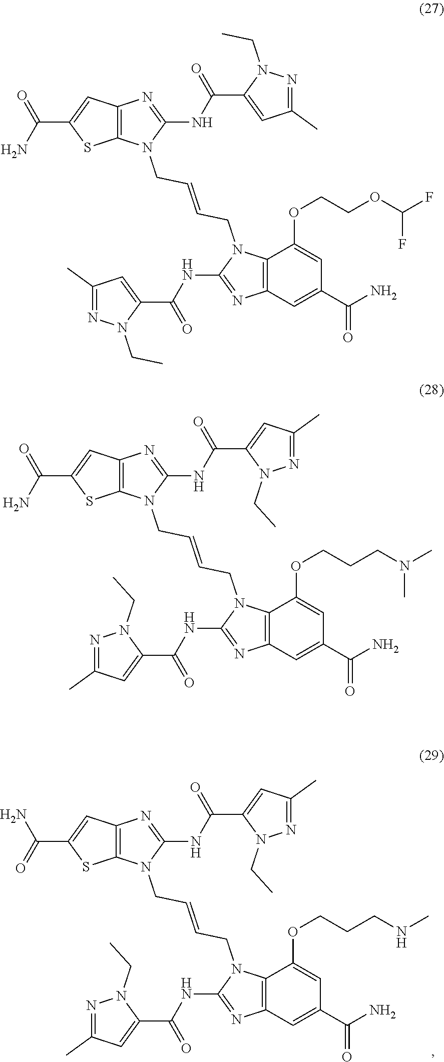 [SMILES: CCn1nc(C)cc1C(=O)Nc1nc2cc(C(N)=O)sc2n1C/C=C/Cn1c(NC(=O)c2cc(C)nn2CC)nc2cc(C(N)=O)cc(OCCOC(F)F)c21 ; CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCN(C)C)c21 ; CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCNC)c21]

or a corresponding salt, solvate, prodrug, isomer thereof, or a combination thereof.

In some cases, the bioactive agent can comprise a compound having Formula (1)

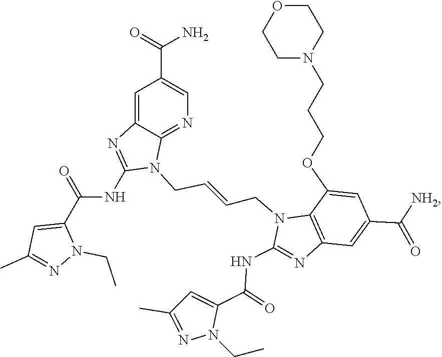 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21]

### or Formula (4)

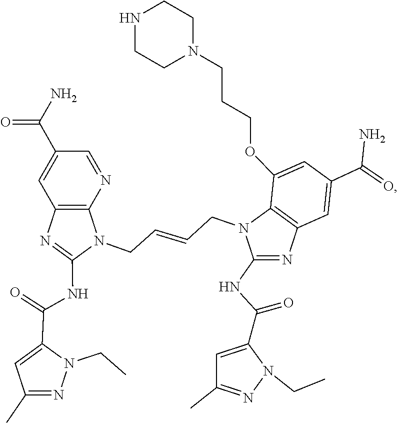 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

a pharmaceutically acceptable salt thereof, solvate thereof, prodrug thereof, isomer thereof, or a combination thereof.

In some cases, the nanoaggregate can be water soluble and can comprise a polymer and at least one bioactive agent comprising at least one STING polypeptide or a part thereof, a nucleic acid encoding said STING polypeptide or a part thereof, a STING inhibitor, a STING activator, a STING agonist, a STING antagonist, a STING modulating molecule, or a combination thereof. The pharmaceutical composition comprising the nanoaggregate can be soluble in an aqueous solution to produce at least 1 mg/mL of the bioactive agent in the aqueous solution. In some cases, the pharmaceutical composition comprising the nanoaggregate can be soluble in an aqueous solution to produce at least 2 mg/mL of the bioactive agent disclosed herein, or a combination thereof, in the aqueous solution. In some cases, the pharmaceutical composition comprising the nanoaggregate can be soluble in an aqueous solution to produce at least 1 mg/ml, 1.5 mg/mL, 2 mg/mL, 2.5 mg/ml, 3 mg/ml, 3.5 mg/mL, 4 mg/mL, 4.5 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, or more, of the bioactive agent disclosed herein, or a combination thereof, in the aqueous solution.

Any polymer disclosed here can be suitable. In some examples, the polymer can comprise a polyoxazoline (POX) that comprises a linear portion, a branched portion, or a combination thereof, and wherein the polyoxazoline (POX) comprises poly(2-oxazoline), poly(2-methyloxazoline), poly(2-ethyloxazoline), poly(2-propyloxazoline), poly(isopropyloxazoline) (PiPOX), or a combination thereof. In some examples, the polyoxazoline can be poly(2-ethyloxazoline).

As used throughout this disclosure, the nanoaggregate can be of a size less than 150 nm before lyophilization. In some cases, the nanoaggregate can be of a size less than 120 nm before lyophilization. By “less than”, is meant size can be less than the defined size in nm and can be about 0 nm, i.e., a solution of the nanoaggregate can be a clear solution with no measurable particles or aggregates. For example, “less than 150 nm” means in a range of from 0 nm to 150 nm and “less than 120 nm” means in a range of from 0 nm to 120 nm. In some cases, the size of the nanoaggregates or nanoparticles, can range from about 0.01 nm to about 100 nm before lyophilization. In some cases, the size of the nanoaggregates or nanoparticles, can range from about 0.01 nm to about 120 nm before lyophilization. In some cases, the size of the nanoaggregates or nanoparticles, can range from about 0.01 nm to about 150 nm before lyophilization. In some cases, the size of the nanoaggregates or nanoparticles, can range from about 50 to about 100 nm before lyophilization. In some cases, the size of the nanoaggregates or nanoparticles, can range from about 50 to about 120 nm before lyophilization. Particle size can be measured by light scattering.

In some cases, the nanoaggregate can be free from human serum albumin, organic solvent, detergent, or oil. In some cases, the nanoaggregate can be free from human serum albumin, organic solvent, detergent, oil or free acid. In some cases, the nanoaggregate can be free from human serum albumin. In some cases, the nanoaggregate can be free from organic solvent. In some cases, the nanoaggregate can be free from detergent. In some cases, the nanoaggregate can be free from oil. In some cases, the nanoaggregate can be free from free acid. In some cases, the nanoaggregate can be free from materials selected from the group consisting of human serum albumin, organic solvent, detergent, oil, free acid, and a combination thereof.

In some cases, the pharmaceutical composition can be free from human serum albumin, organic solvent, detergent, or oil. In some cases, the pharmaceutical composition can be free from human serum albumin, organic solvent, detergent, oil or free acid. In some cases, the pharmaceutical composition can be free from human serum albumin. In some cases, the pharmaceutical composition can be free from organic solvent. In some cases, the pharmaceutical composition can be free from detergent. In some cases, the pharmaceutical composition can be free from oil. In some cases, the pharmaceutical composition can be free from free acid. In some cases, the pharmaceutical composition can be free from materials selected from the group consisting of human serum albumin, organic solvent, detergent, oil, free acid, and a combination thereof.

The pharmaceutical composition of this disclosure can be a drug for treating or preventing a disease selected from immune disorders, infectious diseases, and a combination thereof. Immune disorders can include immunodeficiency disorders, overactive immune disorders, autoimmune diseases, and other disorders or symptoms that have abnormal immune systems. In some cases, the immune disorders can be various autoinflammatory, autoimmune and degenerative diseases, such as those associated with STING (stimulator of interferon genes) signaling pathway or IDO pathway. In some cases, immune disorders can be associated with STING mediated inflammation in infection, cellular stress and tissue damage. In some cases, the pharmaceutical composition of this disclosure can comprise STING protein, STING agonists, STING activators, STING inhibitors, STING antagonists, IDO inhibitors, IDO1 inhibitors, or a combination thereof. In some cases, the pharmaceutical composition of this disclosure can comprise one or more STING inhibitors. In some cases, the pharmaceutical composition of this disclosure can comprise one or more STING activators.

The pharmaceutical composition of this disclosure can be a drug for treating or preventing one or more of the diseases disclosed herein.

In some cases, the pharmaceutical composition can comprise two or more bioactive agents, wherein at least one of the two or more bioactive agents is water insoluble or poorly water soluble. In some cases, at least one of the two or more bioactive agents is at least one STING polypeptide or a part thereof, a nucleic acid encoding said STING polypeptide or a part thereof, a STING inhibitor, a STING activator, a STING agonist, a STING antagonist, a STING modulating molecule, or a combination thereof. In some cases, the pharmaceutical composition can comprise at least one water insoluble or poorly water soluble bioactive agent, in nanoaggregate and one or more additional bioactive agents that are either included in the nanoaggregate or not included in the nanoaggregate. The pharmaceutical composition can comprise a nanoaggregate comprises a polymer and two or more bioactive agents that each is water insoluble or poorly water soluble. The pharmaceutical composition can comprise a nanoaggregate comprises a polymer and at least one bioactive agent comprising at least one STING polypeptide or a part thereof, a nucleic acid encoding said STING polypeptide or a part thereof, a STING inhibitor, a STING activator, a STING agonist, a STING antagonist, a STING modulating molecule, or a combination thereof, and one or more additional bioactive agents that each is water soluble.

The term “a combination thereof” used for a combination of the bioactive agents disclosed above means a combination of two or more bioactive agents, wherein such combination does not have undesired effect, such as, an undesired interaction between or among the bioactive agents. It is understood that some combinations of the bioactive agents may not be suitable, or may not be desirable, such as, those having undesired interactions. For example, a combination of theophylline and ciprofloxacin or warfarin and diflunisal may not be suitable. These combinations or any combinations determined by appropriate guidelines or regulations as not suitable are thus excluded.

As mentioned above, the nanoaggregate can be of a size less than 150 nm before lyophilization. In some cases, the nanoaggregate can be of a size less than 120 nm before lyophilization. In some cases, the nanoaggregate can be of a size in a range of from about 0.01 nm or about 0 nm to about 150 nm before lyophilization. The nanoaggregate can be of a size in a range of from about 50 nm to about 150 nm before lyophilization. In some cases, the size of the nanoaggregates or nanoparticles, can range from about 50 to about 100 nm before lyophilization. In some cases, the size of the nanoaggregates or nanoparticles, can range from about 50 to about 120 nm before lyophilization. In a further example, the nanoaggregate can be of a size in a range of from about from 70 to 90 nm before lyophilization. Particle size can be measured by light scattering.

In any of the pharmaceutical compositions disclosed above and hereafter, the nanoaggregate can have a weight ratio of polymer to bioactive agent in a range of from about 2:1 to about 200:1. The nanoaggregate can have a weight ratio of the polymer to the bioactive agent in a range of from about 2:1 to about 200:1 in one example, about 2:1 to about 150:1 in another example, about 2:1 to about 120:1 in yet another example, about 2:1 to about 100:1 in yet another example, about 2:1 to about 80:1 in yet another example, about 2:1 to about 60:1 in yet another example, about 2:1 to about 40:1 in yet another example, about 2:1 to about 30:1 in yet another example, about 2:1 to about 20:1 in yet another example, about 2:1 to about 15:1 in another example, about 2:1 to about 10:1 in yet another example, about 2:1 to about 8:1 in yet another example, about 5:1 to about 10:1 in yet another example, about 5:1 to about 8:1 in yet another example, about 5:1 in yet a further example, about 6:1 to about 8:1 in yet another example, about 6:1 in yet a further example, about 7:1 in yet a further example, about 7.5:1 in yet a further example, and about 8:1 in yet a further example. When a pharmaceutical composition comprises two or more bioactive agents, the ratio of polymer to bioactive agent can be based on the total weight of polymer and the bioactive agents.

In some cases, the pharmaceutical composition can be an adjuvant for a vaccine. In some cases, the pharmaceutical composition can be an adjuvant for a vaccine for treating or preventing an infectious disease selected from Chickenpox (Varicella), Coronaviruses, Dengue, Diphtheria, Ebola, Flu (Influenza), Hepatitis, Hib Disease, HIV/AIDS, HPV (Human Papillomavirus), Japanese Encephalitis, Measles, Meningococcal Disease, Monkeypox, Mumps, Norovirus, Pneumococcal Disease, Polio, Rabies, Respiratory Syncytial Virus (RSV), Rotavirus, Rubella (German Measles), Shingles (Herpes zoster), Tetanus (Lockjaw), Whooping Cough (Pertussis), Zika, and a combination thereof. In some cases, the pharmaceutical composition can be an adjuvant for a vaccine for treating or preventing an infectious disease caused by 229E α-coronavirus, NL63 α-coronavirus, OC43 β-coronavirus, HKU1 β-coronavirus, MERS-CoV, SARS-CoV, SARS-CoV-2, a variant thereof, or a combination thereof.

In some cases, the pharmaceutical composition can be a prophylactic vaccine, a therapeutic vaccine, or a combination thereof, wherein the pharmaceutical composition can comprise the adjuvant, such as any one of the Adjuvant Formulations disclosed herein, and further comprise at least one immune agent for stimulating an immune response in a subject in need thereof. In some cases, the adjuvant can comprise at least a STING agonist. In some cases, the adjuvant can comprise at least a compound having Formula (1)-Formula (29), a pharmaceutically acceptable salt thereof, solvate thereof, prodrug thereof, isomer thereof, or a combination thereof. In some cases, the adjuvant can be any one Adjuvant Formulation selected from Adjuvant Formulation-1 through Adjuvant Formulation-6 as disclosed hereafter. The immune agent can comprise an inactive microbe selected from bacteria, viruses, fungi, protozoa, worms, parasites, prions, a part thereof, or a combination thereof; toxins; nucleic acids encoding the toxins; proteins; nucleic acids encoding the proteins; oligo nucleic acids; DNAs; RNAs; mRNAs; siRNAs; sgRNAs; fragments thereof; or a combination thereof.

In some cases, the pharmaceutical composition can be formulated for treating or preventing at least one infectious disease. In some cases, the pharmaceutical composition can be formulated for treating or preventing at least one infectious disease selected from Chickenpox (Varicella), Coronaviruses, Dengue, Diphtheria, Ebola, Flu (Influenza), Hepatitis, Hib Disease, HIV/AIDS, HPV (Human Papillomavirus), Japanese Encephalitis, Measles, Meningococcal Disease, Monkeypox, Mumps, Norovirus, Pneumococcal Disease, Polio, Rabies, Respiratory Syncytial Virus (RSV), Rotavirus, Rubella (German Measles), Shingles (Herpes zoster), Tetanus (Lockjaw), Whooping Cough (Pertussis), Zika, and a combination thereof. In some cases, the pharmaceutical composition can be formulated for treating or preventing at least an infectious disease caused by 229E α-coronavirus, NL63 α-coronavirus, OC43 β-coronavirus, HKU1 β-coronavirus, MERS-CoV, SARS-CoV, SARS-CoV-2, a variant thereof, or a combination thereof.

In some cases, the pharmaceutical composition can have a pH value in a range of from 3.0 to 6.9. The pH value can be measured in an aqueous solution of the pharmaceutical composition.

In some cases, the pharmaceutical composition can comprise one or more of the bioactive agents disclosed herein, a derivative thereof, or a combination thereof, wherein in a range of from 1% to 100% of the second terminal group is free from primary amine, and wherein in a range of from 1% to 100% of the second terminal group comprises hydroxyl group.

In some cases, the pharmaceutical composition can further comprise one or more subsequent bioactive agents selected from a protein, a peptide, an antibody, a fragment of an antibody, a chemical compound, a small molecule drug, one or more chemotherapy drugs, and a combination thereof.

The pharmaceutical composition disclosed above and hereafter can further comprise an additional bioactive agent that is formulated free from the polymer, specifically the polymer disclosed herein. The phrase “additional bioactive agent that is formulated free from the polymer” refers to a bioactive agent formulation that comprises a bioactive agent and is free from the polymer disclosed herein, wherein the additional bioactive agent can be a salt, a base, a bioactive agent formulated with organic solvent, detergent, oil or free acid, protein, lipid or a combination thereof. In some cases, the pharmaceutical composition can comprise one or more bioactive agent formulated with aluminum salts, organic adjuvants, or a combination thereof.

The term “soluble in an aqueous solution” refers to a solution that comprises no detectable particles or has particles that can be filtered through a 0.22 μm filter with a filtration rating (R_{f}) through the 0.22 μm filter in a range of from 50 to 100 percent, wherein the filtration rating refers to the volume percent that passes through the filter before the filter becomes clogged resulting in stoppage of filtration. As used throughout this disclosure, the term “0.22 μm filter” refers to a filter assembly having a 0.22 μm filtration pore size. The term “0.8 μm filter” refers to a filter assembly having a 0.8 μm filtration pore size. A combination of 0.8 μm filter and 0.22 μm filter can be suitable.

The pharmaceutical composition disclosed herein can be formulated for parenteral, oral, nasal, transdermal (topical), transmucosal, rectal administration, vaginal administration, or a combination thereof and can comprise one or more pharmaceutically suitable carriers. In some cases, the pharmaceutical composition disclosed herein can be formulated for intravenous (IV), intradermal (ID), subcutaneous (SC), oral, transdermal (topical), transmucosal, rectal administration, or a combination thereof. In some cases, the pharmaceutical composition disclosed herein can be formulated for intravenous (IV), intradermal (ID), subcutaneous (SC), transdermal (topical), nasal spray, inhalation, oral tablet, eye drop, rectal immunization, vaginal immunization, transmucosal administration or a combination thereof. In some cases, the pharmaceutical composition disclosed herein can be formulated for oral administration, such as tablets, capsules, oral spray, solutions, or suspensions. In some cases, the pharmaceutical composition disclosed herein can be formulated for nasal administration, such as, a nasal spray. The pharmaceutically suitable carriers disclosed herein can be suitable.

In some cases, the immune agent can comprise at least a polypeptide of spike (S) glycoprotein of a coronavirus, a DNA encoding said spike (S) glycoprotein, an RNA encoding said spike (S) glycoprotein, a receptor-binding domain (RBD) of said spike (S) glycoprotein, a DNA encoding said RBD, an RNA encoding said RBD, a part thereof, or a combination thereof.

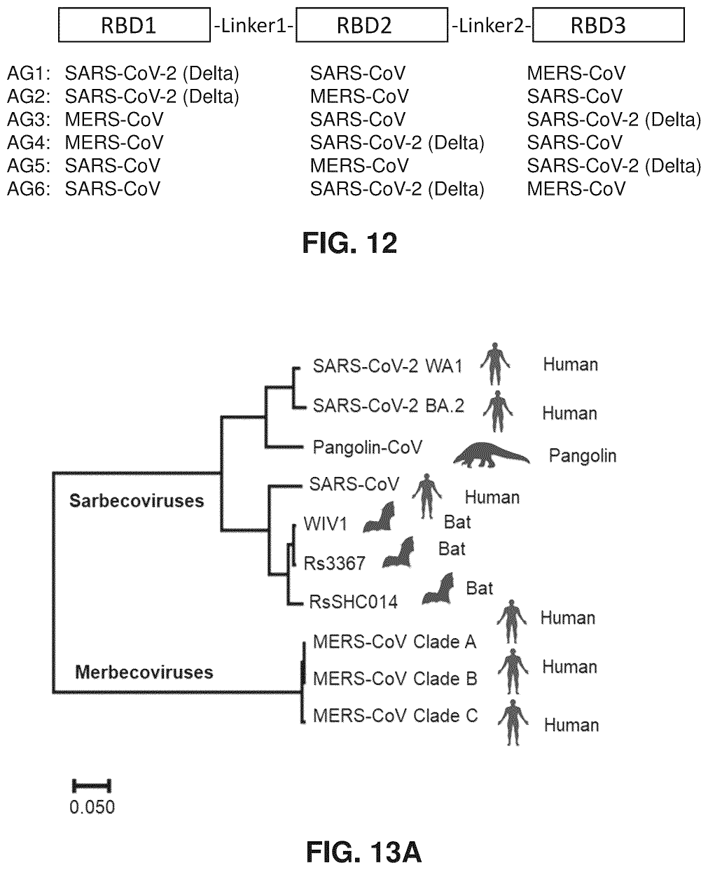

Coronaviruses are a group of RNA viruses that cause disease in mammals and birds including human. Coronavirus can include (1) Genus Alphacoronavirus that includes, Species: Alphacoronavirus 1 (TGEV, Feline coronavirus and Canine coronavirus), Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2 and Scotophilus bat coronavirus 512; (2) Genus Betacoronavirus that includes Species: Betacoronavirus 1 (Bovine Coronavirus and Human coronavirus OC43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-CoV and SARS-CoV-2), and Tylonycteris bat coronavirus HKU4; (3) Genus Gammacoronavirus that includes Species: Avian coronavirus and Beluga whale coronavirus SW1; and (4) Genus Deltacoronavirus that includes Species: Bulbul coronavirus HKU11 and Porcine coronavirus HKU15. An example of a phylogenetic tree of some of the viruses is shown in FIG. 13A.

The coronavirus can comprise 229E (alpha coronavirus), NL63 (alpha coronavirus), OC43 (beta coronavirus), HKU1 (beta coronavirus), MERS-CoV (beta coronavirus that causes Middle East Respiratory Syndrome, or MERS), SARS-CoV (beta coronavirus that causes severe acute respiratory syndrome, or SARS), SARS-CoV-2 (novel coronavirus that causes coronavirus disease 2019 or COVID-19), a variant thereof, or a combination thereof.

In some cases, the coronavirus can comprise 229E alpha coronavirus, NL63 alpha coronavirus, OC43 beta coronavirus, HKU1 beta coronavirus, MERS-CoV, SARS-CoV, SARS-CoV-2, a variant thereof, or a combination thereof.

In some cases, the coronavirus can be selected from 229E alpha coronavirus, NL63 alpha coronavirus, OC43 beta coronavirus, HKU1 beta coronavirus, MERS-CoV, SARS-CoV, SARS-CoV-2, a variant thereof, and a combination thereof.

In some cases, the immune agent can comprise at least a polypeptide of SEQ ID NO:1, SEQ ID NO:2, SEQ ID NO:3, SEQ ID NO:5, SEQ ID NO:6, SEQ ID NO:7, SEQ ID NO:8, SEQ ID NO:9, SEQ ID NO: 12, SEQ ID NO:13, SEQ ID NO:14, SEQ ID NO:15, SEQ ID NO: 16, SEQ ID NO: 17, SEQ ID NO: 18, SEQ ID NO:19, SEQ ID NO:20, SEQ ID NO:21, or a combination thereof. In some cases, the immune agent can comprise two or more of the RBD sequences disclosed thereof. In some cases, the immune agent can comprise three or more of the RBD sequences disclosed thereof.

In some cases, the immune agent can comprise at least a polypeptide of SEQ ID NO:5, SEQ ID NO:6, SEQ ID NO:7, SEQ ID NO:8, or SEQ ID NO:9. In some cases, the immune agent can comprise immune agent selected from SEQ ID NO:5, SEQ ID NO:6, SEQ ID NO:7, SEQ ID NO:8, SEQ ID NO:9, and a combination thereof.

In some cases, a weight ratio of the immune agent:adjuvant can be in a range of from 1:50 to 50:1, wherein the weight ratio is based on the weight of the immune agent and of the bioactive agent as the adjuvant. In some cases, the pharmaceutical composition can comprise a weight ratio of an RBD antigen fusion protein:bioactive agent in a range of from 1:50 to 50:1, wherein the bioactive agent can be selected from Formula (1)-Formula (29), and a combination thereof. In some cases, the pharmaceutical composition can comprise a weight ratio of an RBD antigen fusion protein:bioactive agent in a range of from 1:50 to 50:1, wherein the bioactive agent can be selected from Formula (1), Formula (4), and a combination thereof.

The pharmaceutical composition can further comprise one or more subsequent bioactive agents selected from a protein, a peptide, an antibody, a fragment of an antibody, a chemical compound, a small molecule drug, one or more chemotherapy drugs, and a combination thereof. In some cases, the subsequent bioactive agent can be a vaccine same or different from the pharmaceutical composition disclosed herein. In some cases, the pharmaceutical composition can be a vaccine treating or preventing a disease caused by Chickenpox (Varicella), Coronaviruses, Dengue, Diphtheria, Ebola, Flu (Influenza), Hepatitis, Hib Disease, HIV/AIDS, HPV (Human Papillomavirus), Japanese Encephalitis, Measles, Meningococcal Disease, Monkeypox, Mumps, Norovirus, Pneumococcal Disease, Polio, Rabies, Respiratory Syncytial Virus (RSV), Rotavirus, Rubella (German Measles), Shingles (Herpes zoster), Tetanus (Lockjaw), Whooping Cough (Pertussis), Zika, or a combination thereof and the subsequent bioactive agent can be a same or different vaccine. In some cases, the pharmaceutical composition can be a vaccine treating or preventing a disease caused by coronavirus and the subsequent bioactive agent can be a vaccine or a vaccine for treating or preventing Chickenpox (Varicella), Coronaviruses, Dengue, Diphtheria, Ebola, Flu (Influenza), Hepatitis, Hib Disease, HIV/AIDS, HPV (Human Papillomavirus), Japanese Encephalitis, Measles, Meningococcal Disease, Monkeypox, Mumps, Norovirus, Pneumococcal Disease, Polio, Rabies, Respiratory Syncytial Virus (RSV), Rotavirus, Rubella (German Measles), Shingles (Herpes zoster), Tetanus (Lockjaw), Whooping Cough (Pertussis), Zika, or a combination thereof.

In some cases, this disclosure is further directed to a method for treating or preventing a disease of a subject in need thereof, the method can comprise administering the subject with an effective dose of a pharmaceutical composition comprising:

a nanoaggregate comprising a polymer and at least one bioactive agent comprising at least one STING polypeptide or a part thereof, a nucleic acid encoding said STING polypeptide or a part thereof, a STING inhibitor, a STING activator, a STING agonist, a STING antagonist, a STING modulating molecule, or a combination thereof; and
optionally a pharmaceutically suitable carrier;
wherein the pharmaceutical composition is soluble in an aqueous solution to produce at least 1 mg/mL of the bioactive agent in the aqueous solution;
wherein the polymer is water soluble; and
wherein the polymer can comprise:
a first polymer comprising at least one first terminal group modified with H or a hydrophobic moiety and a second terminal group modified with a hydrophilic moiety, wherein the first terminal group comprises in a range of from 1% to 100% of H and 0% to 99% of the hydrophobic moiety that comprises a saturated or an unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof; or
a second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or
a combination thereof.

In some cases, the polymer can comprise the first polymer, as disclosed herein.

In some cases, the polymer can consist of the first polymer, as disclosed herein. In some cases, the pharmaceutical composition can be free from polymers selected from one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof.

In some cases, the polymer can comprise one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or a combination thereof.

In some cases, the polymer can comprise the first polymer and one or more subsequent polymers (also referred to as “second polymer”) selected from one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof.

The bioactive agents disclosed herein can be suitable.

Any polymers disclosed herein can be suitable. In some cases, the polymer can comprise a polyoxazoline (POX) that comprises a linear portion, a branched portion, or a combination thereof, and wherein the polyoxazoline (POX) comprises poly(2-oxazoline), poly(2-methyloxazoline), poly(2-ethyloxazoline), poly(2-propyloxazoline), poly(isopropyloxazoline), or a combination thereof. In some cases, the polyoxazoline is poly(2-ethyloxazoline).

As mentioned above, the nanoaggregate can be of a size less than 150 nm before lyophilization. In some cases, the nanoaggregate can be of a size less than 120 nm before lyophilization. In some cases, the nanoaggregate can be of a size in a range of from about 0.01 nm or about 0 nm to about 150 nm before lyophilization. The nanoaggregate can be of a size in a range of from about 50 nm to about 150 nm before lyophilization. In some cases, the size of the nanoaggregates or nanoparticles, can range from about 50 to about 100 nm before lyophilization. In some cases, the size of the nanoaggregates or nanoparticles, can range from about 50 to about 120 nm before lyophilization. In a further example, the nanoaggregate can be of a size in a range of from about from 70 to 90 nm before lyophilization. Particle size can be measured by light scattering.

In some cases, the nanoaggregate can have a weight ratio of the polymer to bioactive agent in a range of from about 2:1 to about 20:1. In some cases, the nanoaggregate can have a weight ratio of the polymer to bioactive agent in a range of from about 5:1 to about 8:1.

In some cases, the nanoaggregate can be free from human serum albumin, organic solvent, detergent, or oil. In some cases, the nanoaggregate can be free from human serum albumin, organic solvent, detergent, oil or free acid. In some cases, the nanoaggregate can be free from human serum albumin. In some cases, the nanoaggregate can be free from organic solvent. In some cases, the nanoaggregate can be free from detergent. In some cases, the nanoaggregate can be free from oil. In some cases, the nanoaggregate can be free from free acid. In some cases, the nanoaggregate can be free from materials selected from the group consisting of human serum albumin, organic solvent, detergent, oil, free acid, and a combination thereof.

In some cases, the pharmaceutical composition can be free from human serum albumin, organic solvent, detergent, or oil. In some cases, the pharmaceutical composition can be free from human serum albumin, organic solvent, detergent, oil or free acid. In some cases, the pharmaceutical composition can be free from human serum albumin. In some cases, the pharmaceutical composition can be free from organic solvent. In some cases, the pharmaceutical composition can be free from detergent. In some cases, the pharmaceutical composition can be free from oil. In some cases, the pharmaceutical composition can be free from free acid. In some cases, the pharmaceutical composition can be free from materials selected from the group consisting of human serum albumin, organic solvent, detergent, oil, free acid, and a combination thereof.

In some cases of the method disclosed herein, in a range of from 1% to 100% of the second terminal group, is free from primary amine. In some cases, in a range of from 1% to 100% of the second terminal group comprises a hydroxyl group. All percentages are based on the total number of the second terminal group.

In some cases, the pharmaceutical composition can have a pH value in a range of from 3.0 to 6.9.

In some cases, suitable to the method, the bioactive agent can comprise at least a compound having Formula (1)-Formula (29) (FIG. 11A-FIG. 11E), a pharmaceutically acceptable salt thereof, solvate thereof, prodrug thereof, isomer thereof, or a combination thereof.

In some cases, the bioactive agent can comprise a compound having Formula (1)

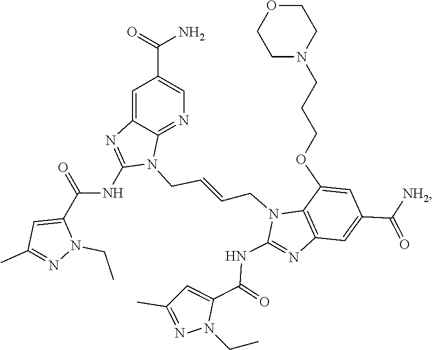 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21]

### or Formula (4)

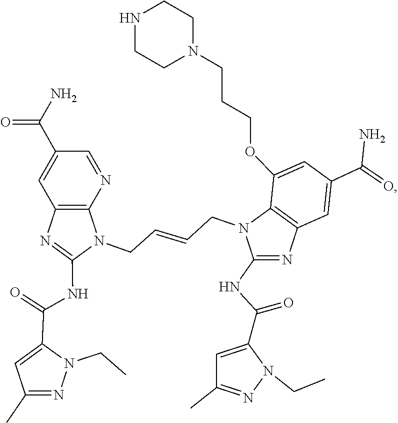 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

or a pharmaceutically acceptable salt thereof, solvate thereof, prodrug thereof, isomer thereof, or a combination thereof.

In some cases, the disease can be selected from immune disorders, infectious diseases, and a combination thereof.

In some cases, immune disorders can include an aforementioned immunodeficiency disorders, overactive immune disorders, autoimmune diseases, and other disorders or symptoms that have abnormal immune systems.

The pharmaceutical composition can be administered to the subject via intravenous (IV) injection, subcutaneous (SC) injection, intramuscular (IM) injection, intradermal (ID) injection, nasal spray, inhalation, oral tablet, eye drop, rectal immunization, vaginal immunization, or a combination thereof.

In some cases, the pharmaceutical composition can be an adjuvant for, for example, a vaccine. In some cases, the pharmaceutical composition is a prophylactic vaccine, a therapeutic vaccine, or a combination thereof, wherein the pharmaceutical composition can comprise the adjuvant and can further comprise at least one immune agent for stimulating immune response in a subject in need thereof. In some cases, the pharmaceutical composition is selected for treating or preventing at least one infectious disease selected from Chickenpox (Varicella), Coronaviruses, Dengue, Diphtheria, Ebola, Flu (Influenza), Hepatitis, Hib Disease, HIV/AIDS, HPV (Human Papillomavirus), Japanese Encephalitis, Measles, Meningococcal Disease, Monkeypox, Mumps, Norovirus, Pneumococcal Disease, Polio, Rabies, Respiratory Syncytial Virus (RSV), Rotavirus, Rubella (German Measles), Shingles (Herpes zoster), Tetanus (Lockjaw), Whooping Cough (Pertussis), Zika, and a combination thereof.

The immune agent can comprise at least a polypeptide of a spike (S) glycoprotein of a coronavirus, a DNA encoding said spike (S) glycoprotein, an RNA encoding said spike (S) glycoprotein, a receptor-binding domain (RBD) of said spike (S) glycoprotein, a DNA encoding said RBD, an RNA encoding said RBD, a part thereof, or a combination thereof.

In some cases, the immune agent can comprise at least one receptor-binding domain (RBD) of a spike (S) glycoprotein of 229E alpha coronavirus, NL63 alpha coronavirus, OC43 beta coronavirus, HKU1 beta coronavirus, MERS-CoV, SARS-CoV, SARS-CoV-2, a variant thereof, or a combination thereof.

In some cases, the immune agent can comprise at least a polypeptide of SEQ ID NO:1, SEQ ID NO:2, SEQ ID NO:3, SEQ ID NO:5, SEQ ID NO:6, SEQ ID NO:7, SEQ ID NO:8, SEQ ID NO:9, SEQ ID NO: 12, SEQ ID NO:13, SEQ ID NO: 14, SEQ ID NO: 15, SEQ ID NO: 16, SEQ ID NO: 17, SEQ ID NO: 18, SEQ ID NO: 19, SEQ ID NO:20, SEQ ID NO:21, or a combination thereof. In some cases, the immune agent can comprise at least a polypeptide of SEQ ID NO: 5, SEQ ID NO:6, SEQ ID NO:7, SEQ ID NO:8, or SEQ ID NO:9.

In some cases, a weight ratio of immune agent:adjuvant can be in a range of from 1:50 to 50:1, wherein the weight ratio is based on the weight of the immune agent and of the bioactive agent acting as the adjuvant. In some cases, suitable to the method, the pharmaceutical composition can comprise a weight ratio of an RBD antigen fusion protein:bioactive agent in a range of from 1:50 to 50:1, wherein the bioactive agent can be selected from Formula (1)-Formula (29), and a combination thereof. In some cases, the pharmaceutical composition can comprise a weight ratio of an RBD antigen fusion protein:bioactive agent in a range of from 1:50 to 50:1, wherein the bioactive agent can be selected from Formula (1), Formula (4), and a combination thereof.

The method can further comprise the step of administering the subject with one or more subsequent bioactive agents selected from a protein, a peptide, an antibody, a fragment of an antibody, a chemical compound, a small molecule drug, one or more chemotherapy drugs, a vaccine, and a combination thereof, wherein each of the one or more subsequent bioactive agents can be administered to the subject, prior to, at the same time as, or after administering said pharmaceutical composition. In some cases, the subsequent bioactive agent can be a vaccine different from the pharmaceutical composition. In some cases, the pharmaceutical composition can be a vaccine treating or preventing a disease caused by coronavirus and the subsequent bioactive agent can be a vaccine or a vaccine for treating or preventing Chickenpox (Varicella), Coronaviruses, Dengue, Diphtheria, Ebola, Flu (Influenza), Hepatitis, Hib Disease, HIV/AIDS, HPV (Human Papillomavirus), Japanese Encephalitis, Measles, Meningococcal Disease, Monkeypox, Mumps, Norovirus, Pneumococcal Disease, Polio, Rabies, Respiratory Syncytial Virus (RSV), Rotavirus, Rubella (German Measles), Shingles (Herpes zoster), Tetanus (Lockjaw), Whooping Cough (Pertussis), Zika, or a combination thereof. Commercial vaccines and the vaccines listed by US Centers for Disease Control and Prevention (CDC) can be suitable as subsequent bioactive agents.

A subsequent bioactive agent described herein can include any chemical or small molecule drug, chemotherapy drugs, inorganic-based drug, biological or large molecule-based drug, modifications or derivatives thereof, and combinations thereof.

In some cases, the pharmaceutical composition can comprise the adjuvant disclosed herein and an immune agent, wherein the adjuvant is administered to a subject in need thereof before, at the same time, or after an immune agent is administered to the subject. In some cases, the pharmaceutical composition can comprise an adjuvant comprising Formula (1)-(29), or a combination thereof, wherein the adjuvant can be administered to a subject in need thereof before, together (co-admixed or co-formulated), or after an immune agent, such as, a commercially available vaccine, a DNA, an mRNA, or a protein, such as, an RBD antigen fusion protein disclosed herein. In some cases, the adjuvant can be administered in a range of 1 minute to 1 day, 5 days, 10 days, 21 days, 30 days, 60 days, 90 days or 120 days prior to administration of an immune agent. In some cases, the adjuvant can be administered simultaneously with an immune agent. In some cases, the adjuvant can be administered in a range of 1 minute to 1 day, 5 days, 10 days, 21 days, 30 days, 60 days, 90 days or 120 days after administration of an immune agent.

In some cases, an adjuvant of the instant disclosure that comprises a polymer and a compound of Formula (1)-(29), or a combination thereof, can be administered to a subject in need thereof before, together (co-admixed or co-formulated), or after administration of a vaccine for treating or preventing a disease selected from Chickenpox (Varicella), Coronaviruses, Dengue, Diphtheria, Ebola, Flu (Influenza), Hepatitis, Hib Disease, HIV/AIDS, HPV (Human Papillomavirus), Japanese Encephalitis, Measles, Meningococcal Disease, Monkeypox, Mumps, Norovirus, Pneumococcal Disease, Polio, Rabies, Respiratory Syncytial Virus (RSV), Rotavirus, Rubella (German Measles), Shingles (Herpes zoster), Tetanus (Lockjaw), Whooping Cough (Pertussis), Zika, or a combination thereof.

In some cases, the vaccine of the instant disclosure that comprises a polymer, a compound of Formula (1)-(29), or a combination thereof, and an immune agent comprising at least a polypeptide of SEQ ID NO:1, SEQ ID NO:2, SEQ ID NO:3, SEQ ID NO:5, SEQ ID NO:6, SEQ ID NO:7, SEQ ID NO:8, SEQ ID NO: 9, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO:20, SEQ ID NO: 21, or a combination thereof, can be administered to a subject in need thereof before, together (co-admixed or co-formulated), or after the administration of a vaccine for treating or preventing a disease selected from Chickenpox (Varicella), Coronaviruses, Dengue, Diphtheria, Ebola, Flu (Influenza), Hepatitis, Hib Disease, HIV/AIDS, HPV (Human Papillomavirus), Japanese Encephalitis, Measles, Meningococcal Disease, Monkeypox, Mumps, Norovirus, Pneumococcal Disease, Polio, Rabies, Respiratory Syncytial Virus (RSV), Rotavirus, Rubella (German Measles), Shingles (Herpes zoster), Tetanus (Lockjaw), Whooping Cough (Pertussis), Zika, or a combination thereof.

In some cases, a method can further comprise the step of administering the subject with one or more booster injections of a pharmaceutical composition, for example, an adjuvant or a vaccine disclosed herein. In some cases, a method can comprise the step of administering one or more booster injections with an adjuvant. In some cases, a method can comprise the step of administering one or more booster injections with a vaccine that comprises an adjuvant and one or more immune agents. In some cases, a method can comprise the step of administering the subject with one or more booster injections with an adjuvant comprising Formula (1)-(29), or a combination thereof and the RBD antigen fusion protein disclosed herein,

A subsequent bioactive composition can be administered with intravenous (IV), intramuscular (IM), subcutaneous (SC) or intradermal (ID) injections, orally, through inhalation, nasally, through an eye, for example, using drops or an ointment, transdermally, for example, using a patch, or a combination thereof. A combination of any of aforementioned administering routes can also be suitable.

Suitable to the method, the pharmaceutical composition can have a weight ratio of the immune agent:adjuvant in a range of from 1:50 to 50:1, wherein the weight ratio is based on the weight of immune agent and of bioactive agent as the adjuvant.

As disclosed above and hereafter, the nanoaggregates can be linked with a targeting moiety or group including, but not limited to, an antibody (or antigen-binding portion thereof), antigen, cognate carbohydrates (e.g., sialic acid), a cell surface receptor ligand, a moiety that binds a cell surface receptor, such as, prostate-specific membrane antigen (PSMA), a moiety that binds a cell surface saccharide, an extracellular matrix ligand, a cytosolic receptor ligand, a growth factor, a cytokine, an incretin, a hormone, a lectin, a lectin target, such as, a galactose, a galactose derivative, an N-acetylgalactosamine, a mannose, a mannose derivative and the like, a vitamin, such as, a folate, a biotin and the like, an avidin, a streptavidin, a neutravidin, etc., to form a conjugate so that the targeting group(s) are incorporated with a nanocomposite particle of interest (FIG. 10A-10B).

In some cases, the bioactive agent can be dissolved in methanol or ethanol in various amounts of up to 40 mg/mL. A hydrocarbon (CH₃(CH₂)₁₇)-modified randomly branched PEOX60 (monomer to initiator molar ratio=60:1) (herein referred to as C₁₈PEOXABP60) can be prepared as taught in PCT Publication No.: WO2014/123791 (the entire disclosure of which is incorporated herein by reference in entirety) and dissolved at varying concentrations of up 100 mg/mL in methanol or ethanol. The two solutions then can be mixed in various volumes to result in final homopolymer to bioactive agent weight ratios in the mixtures ranging from 2:1 to 20:1 to form the nanoaggregate. The nanoaggregate can be rotary evaporated to dryness to form a dried nanoaggregate. The dried nanoaggregate then can be re-dissolved in water or saline, followed by sterile filtration with a 0.22 μm filter and lyophilization for 20 to 72 hours depending on volume to yield a lyophilized nanoaggregate as a dry powder.

In some cases, a polymer mixture H/C₁₈PEOXABP60 can be suitable. The polymer mixture H/C₁₈PEOXABP60 can comprise polymers having the second terminal group modified by —OH, NH₂ or a combination thereof.

A pharmaceutical composition comprising the nanoaggregate disclosed herein can be formulated to be compatible with the intended administering route and can comprise one or more pharmaceutically suitable carriers. Examples of routes of administration include parenteral, e.g., intravenous (IV), intradermal (ID), subcutaneous (SC), oral (e.g., inhalation), transdermal (topical), transmucosal and rectal administration. Solutions or suspensions used for parenteral, intradermal or subcutaneous application can include one or more pharmaceutically suitable carriers, such as a sterile diluent, such as, water for injection, saline, oils, polyethylene glycols, glycerine, propylene glycol or other synthetic solvents; antibacterial agents, such as, benzyl alcohol or methyl parabens; antioxidants, such as, ascorbic acid or sodium bisulfite; chelating agents, such as, EDTA; buffers, such as, acetates, citrates or phosphates; and agents for the adjustment of tonicity, such as, sodium chloride or dextrose. pH can be adjusted with acids or bases, such as, HCl or NaOH. The parenteral preparation can be enclosed in ampoules, disposable syringes or multiple dose vials made of glass or plastic as an article of manufacture. The pharmaceutical composition can be packaged in a container, pack or dispenser together with instructions for administration.

The pharmaceutical compositions can be suitable for injectable use and can include sterile aqueous solutions or dispersions and sterile powders for the extemporaneous preparation of sterile injectable solutions or dispersions. For intravenous administration, suitable carriers can include physiological saline, bacteriostatic water, Cremophor ER (BASF; Parsippany, NJ) or phosphate-buffered saline (PBS). The composition is sterile and is fluid to the extent that syringability exists. The composition must be stable under the conditions of manufacture and storage and must be preserved against the contaminating action of microorganisms, such as, bacteria and fungi. The pharmaceutical composition can comprise one or more solvents or dispersion medium containing, for example, water, ethanol, polyol (for example, glycerol, propylene glycol, liquid PEG, polysorbates and the like) and suitable mixtures thereof. Some pharmaceutically suitable carriers can be used for maintaining proper fluidity of the composition, for example, by use of a coating, such as, lecithin, by maintenance of the required particle size in the case of a dispersion, use of a thickener and use of surfactants. Further pharmaceutically suitable carriers can include various antibacterial and antifungal agents, for example, parabens, chlorobutanol, phenol, ascorbic acid and the like, to prevent or to inhibit growth or action of microorganisms; and isotonic agents, for example, sugars, polyalcohols, such as, mannitol, sorbitol or sodium chloride, can be included in the composition as a pharmaceutically suitable carrier. An agent that delays absorption, for example, aluminum monostearate or gelatin, can also be used as a pharmaceutically suitable carrier.

In a further embodiment, the pharmaceutical composition can comprise one or more pharmaceutically suitable carriers that will protect the compound against rapid elimination from the body of a subject, such as, a controlled release formulation, including implants and microencapsulated delivery systems. Biodegradable, biocompatible polymers can be used, such as, ethylene vinyl acetate, polyanhydrides, polyglycolic acid, collagen, polyorthoesters and polylactic acid. Methods for preparation of such formulations will be apparent to those skilled in the art. The materials also can be obtained commercially, for example, from Alza Corporation and Nova Pharmaceuticals, Inc.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches or capsules. Oral compositions also can be prepared using a fluid carrier to yield a syrup or liquid formulation, or for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients or compounds of a similar nature: a binder, such as, microcrystalline cellulose, gum tragacanth or gelatin; an excipient, such as, starch or lactose; a disintegrating agent, such as, alginic acid, Primogel or corn starch; a lubricant, such as, magnesium stearate or Sterotes; a glidant, such as, colloidal silicon dioxide; a sweetening agent, such as, sucrose or saccharin; or a flavoring agent, such as, peppermint, methyl salicylate or orange flavoring.

For administration by inhalation, the compound is delivered in the form of, for example, an aerosol spray from a pressurized container or dispenser that contains a suitable propellant, e.g., a gas, such as, carbon dioxide; a nebulizer; or a mister.

Systemic administration also can be by transmucosal or transdermal means. For transmucosal or transdermal administration, penetrants appropriate to the barrier to be permeated are used in the formulation. Such penetrants generally are known in the art and include, for example, for transmucosal administration, detergents, bile salts and fusidic acid derivatives. Transmucosal administration can be accomplished through the use of nasal sprays or suppositories. For transdermal administration, the active compounds are formulated into ointments, salves, gels or creams as generally known in the art. Another known penetrant is dimethyl sulfoxide.

The compound also can be prepared in the form of suppositories (e.g., with conventional suppository bases, such as, cocoa butter and other glycerides) or retention enemas for rectal delivery.

It can be advantageous to formulate oral or parenteral compositions in dosage unit form for ease of administration and uniformity of dosage. Dosage unit form as used herein refers to physically discrete units suited as unitary dosages for a subject to be treated; each unit containing a predetermined quantity of active compound calculated to produce a desired therapeutic endpoint. The dosages, for example, preferred route of administration and amounts are obtainable based on empirical data obtained from preclinical and clinical studies, practicing methods known in the art. The dosage and delivery form can be dictated by and can be dependent on the characteristics of the bioactive agent, the polymer, the particular therapeutic effect to be achieved, the characteristics and condition of the recipient and so on. For repeated administrations over several days or longer, depending on the condition, the treatment can be sustained until a desired endpoint is attained.

In some cases, this disclosure is directed to a nanoaggregate comprising a polymer and at least one bioactive agent comprising at least one STING polypeptide or a part thereof, a nucleic acid encoding said STING polypeptide or a part thereof, a STING inhibitor, a STING activator, a STING agonist, a STING antagonist, a STING modulating molecule, or a combination thereof;
wherein the nanoaggregate is soluble in an aqueous solution to produce at least 1 mg/mL of the bioactive agent in the aqueous solution;
wherein the polymer is water soluble; and
wherein the polymer comprises:
a first polymer comprising at least one first terminal group modified with H or a hydrophobic moiety and a second terminal group modified with a hydrophilic moiety, wherein the first terminal group comprises in a range of from 1% to 100 of H and 0% to 99% of the hydrophobic moiety that comprises a saturated or an unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof; or
a second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or a combination thereof.

In some cases, in a range of from about 1% to 100% of the second terminal group can be free from primary amine. In some cases, the nanoaggregate disclosed herein, in a range of from 1% to 100% of the second terminal group can comprise a hydroxyl group. All percentages are based on the total number of second terminal groups.

In some cases, a nanoaggregate can be of a size less than 150 nm before lyophilization. In some cases, the nanoaggregate can be of a size less than 120 nm before lyophilization. In some cases, the nanoaggregate can be of a size in a range of from about 0.01 nm or about 0 nm to about 150 nm before lyophilization as described before. In some cases, the nanoaggregate can be of a size in a range of from about 50 nm to about 120 nm before lyophilization.

In some cases, poly(2-ethyloxazoline) can comprise a molar ratio of monomer to initiator in a range of from 50:1 to 80:1.

In some cases, a nanoaggregate can have a weight ratio of polymer to bioactive agent in a range of from about 2:1 to about 200:1. In some cases, a nanoaggregate can have a weight ratio of polymer to bioactive agent in a range of from about 5:1 to about 8:1.

A nanoaggregate can be free from human serum albumin, organic solvent, detergent, or oil. A nanoaggregate can be free from human serum albumin, organic solvent, detergent, oil or free acid.

In some cases, suitable to a nanoaggregate disclosed herein, a bioactive agent can comprise at least a compound having Formula (1)-Formula (29) (FIG. 11A-FIG. 11E), a pharmaceutically acceptable salt thereof, solvate thereof, prodrug thereof, isomer thereof, or a combination thereof.

In some cases, a bioactive agent can comprise a compound having Formula (1):

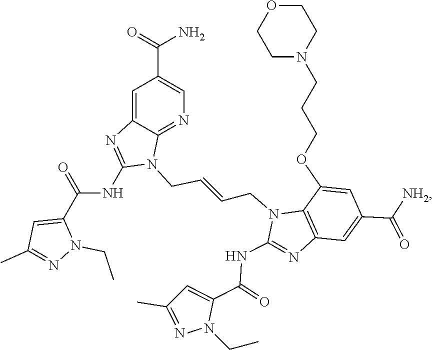 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21]

### or Formula (4)

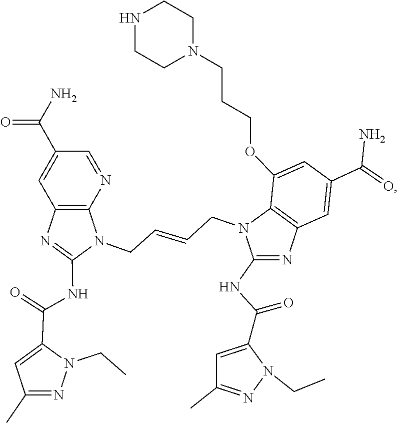 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

or a pharmaceutically acceptable salt thereof, solvate thereof, prodrug thereof, isomer thereof, or a combination thereof.

This invention is further directed to a use of nanoaggregates comprising a polymer and at least one bioactive agent comprising at least one STING polypeptide or a part thereof, a nucleic acid encoding said STING polypeptide or a part thereof, a STING inhibitor, a STING activator, a STING agonist, a STING antagonist, a STING modulating molecule, or a combination thereof, and optionally a pharmaceutically suitable carrier, for manufacturing a medicament for treatment of a disease;
wherein the disease is selected from immune disorders, infectious diseases, and a combination thereof;
wherein the nanoaggregates are soluble in an aqueous solution to produce at least 1 mg/mL of bioactive agent in the aqueous solution;
wherein the polymer is water soluble; and
wherein the polymer comprises:
a first polymer comprising at least one first terminal group modified with H or a hydrophobic moiety and a second terminal group modified with a hydrophilic moiety, wherein the first terminal group comprises in a range of from 1% to 100% of H and 0% to 99% of the hydrophobic moiety that comprises a saturated or an unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof; or
a second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or
a combination thereof.

In some cases, the polymer can comprise the first polymer as disclosed herein.

In some cases, a polymer can consist of the first polymer, as disclosed herein. In some cases, a pharmaceutical composition can be free from polymers selected from one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof.

In some cases, in a range of from about 1% to 100%, second terminal group can be free from a primary amine. In some cases, the nanoaggregate disclosed herein, in a range of from 1% to 100% of second terminal group can comprise a hydroxyl group. All percentages are based on total number of second terminal groups.

In some cases, the polymer can comprise one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or a combination thereof.

In some cases, a polymer can comprise a first polymer and one or more of subsequent polymers (also referred to as “second polymer”) selected from one or more h dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); Pluronics® (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); and a combination thereof.

A bioactive agent can comprise at least a compound of Formula (1)-Formula (29) (FIG. 11A-FIG. 11E), a pharmaceutically acceptable salt thereof, solvate thereof, prodrug thereof, isomer thereof, or a combination thereof.

In some cases, the bioactive agent can comprise a compound having Formula (1):

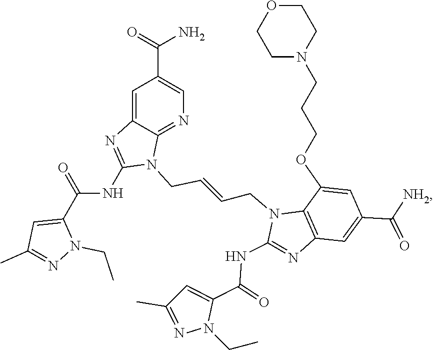 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21]

or

Formula (4)

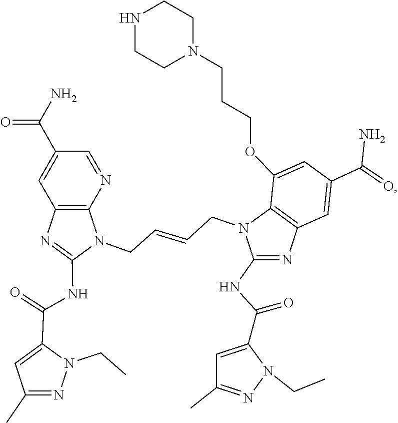 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

or a pharmaceutically acceptable salt thereof, solvate thereof, prodrug thereof, isomer thereof, or a combination thereof.

In some cases, the disease can comprise immune disorders, infectious diseases, and a combination thereof.

In some cases, the polymer can comprise a polyoxazoline (POX) that comprises a linear portion, a branched portion, or a combination thereof, and wherein the polyoxazoline (POX) comprises poly(2-oxazoline), poly(2-methyloxazoline), poly(2-ethyloxazoline), poly(2-propyloxazoline), poly(isopropyloxazoline), or a combination thereof.

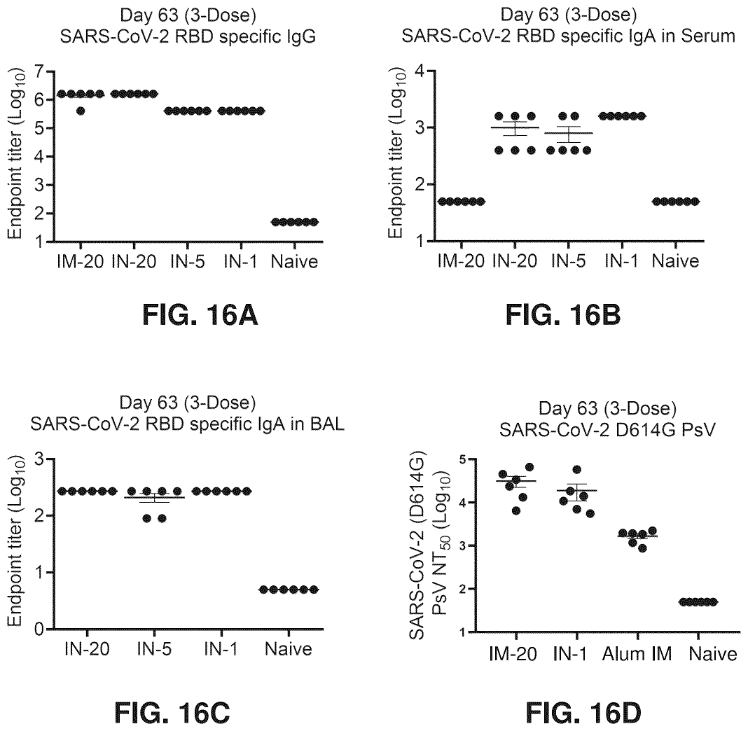

Applicants unexpectedly discovered that an adjuvant comprising a polymer disclosed herein and a compound having Formula (1)-(29) (“Nano-Adjuvant”) significantly enhances antigenicity of a broad spectrum of immune agents. In some cases, the Nano-Adjuvant can enhance immune response compared to traditional aluminum-containing adjuvant. In some cases, the Nano-Adjuvant can enhance the production of neutralization antibodies compared to the traditional aluminum-containing adjuvant (FIG. 16D).

The instant disclosure now will be exemplified in the following non-limiting examples.

### EXAMPLES

The present invention is further defined in the following Examples. It should be understood that these Examples, while indicating preferred embodiments of the invention, are given by way of illustration only. From the above discussion and the Examples, one skilled in the art can ascertain the essential characteristics of the instant invention, and without departing from the spirit and scope thereof, can make various changes and modifications of the invention to adapt same to various uses and conditions.

### Materials and Measurements

### Polymers

Hydrocarbon (CH₃(CH₂)₁₇)-modified randomly branched PEOX polymer having a monomer to initiator molar ratio=60:1 (herein “C₁₈PEOXABP60”) was prepared using the initiator, (CH₃(CH₂)₁₇)—Br, as described previously in PCT Publication No.: WO2014/123791, herein incorporated by reference in entirety.

Another hydrocarbon modified randomly branched PEOX polymer having a monomer to initiator molar ratio=60:1 was prepared as described above with the initiator, (CH₃(CH₂)₅)—Br.

Another hydrocarbon modified randomly branched PEOX polymer having a monomer to initiator molar ratio=60:1 was prepared as described above with then initiator, (CH₃(CH₂)₁₁)—Br.

Another hydrocarbon modified randomly branched PEOX polymer having a monomer to initiator molar ratio=60:1 was prepared as described above with the initiator, methyl tosylate.

Hydrogen modified randomly branched PEOX polymer having a monomer to initiator molar ratio=60:1 (herein “H-PEOXABP60”) was prepared as described above with the initiator, p-toluenesulfonic acid.

Another hydrogen modified randomly branched PEOX polymer having a monomer to initiator molar ratio=60:1 was prepared as described above with the initiator, trifluoroacetic acid.

The hydrogen modified randomly branched PEOX polymers having a monomer to initiator molar ratio=60:1 are collectively referred to as “H-PEOXABP60”.

Table 1 shows some non-limiting examples of polymers with various first terminals modified with H (H-PEOXABP60) and C₁₈ hydrocarbon (C₁₈PEOXABP60). The presence of the H and the C₁₈ hydrocarbon modified first terminal groups were measured with HPLC. Molar ratios of H to C₁₈ hydrocarbon and percent of H are shown.

TABLE 1
Ratios of H to C₁₈ hydrocarbon and percent of H of Mixed Polymer
Examples H-PEOXABP60 and C₁₈PEOXABP60 (Molar ratio).

| | H to C18 Ratio | % of H |
|---|---|---|
| Polymers | (H/C₁₈ Molar Ratio) | (Molar Percent) |
| Polymer 1 | 0.10 | 9% |
| Polymer 2 | 0.33 | 25% |
| Polymer 3 | 0.52 | 34% |
| Polymer 4 | 0.65 | 39% |
| Polymer 5 | 0.76 | 43% |
| Polymer 6 | 0.99 | 50% |
| Polymer 7 | 1.02 | 50% |
| Polymer 8 | 1.49 | 60% |

Polymers comprising a mixture of hydrocarbon (CH₃(CH₂)₁₇)-modified first terminals and H modified first terminals with an initiator molar ratio=60:1, herein “H/C₁₈PEOXABP60”, were used for producing the nanoaggregates.

A polymer, H/C₁₈PEOXABP60, having an H/C₁₈ of about 0.3 was terminated in water with the hydroxyl group as the second terminal group (herein referred to as “Polymer A1”). Aqueous solutions of Polymer A1 had pH values in a range of from 4.0 to 6.9. If needed, an aqueous solution of Polymer A1 can be adjusted to have pH values in a range of from 5.6 to 7.5 or 4.0 to 10 using HCl or NaOH.

A polymer, H/C₁₈PEOXABP60, having an H/C₁₈ of about 0.4 was terminated in water with a hydroxyl group as the second terminal group (herein referred to as “Polymer A2”). An aqueous solution of Polymer A2 can be adjusted to have pH values in a range of from 4.0 to 6.9. Another aqueous solution of Polymer A2 can be adjusted to have pH values in a range of from 7.0 to 10 or 5.6 to 7.5 using HCl or NaOH.

A polymer, H/C₁₈PEOXABP60, having an H/C₁₈ of about 0.7 was terminated in water with a hydroxyl group as the second terminal group (herein referred to as “Polymer A3”). Aqueous solutions of Polymer A3 had pH values in a range of from 4.0 to 6.9. If needed, an aqueous solution of Polymer A3 can be adjusted to have pH values in a range of from 7.0 to 10 or 5.6 to 7.5 using HCl or NaOH.

All H/C₁₈PEOXABP60 polymers having a hydroxyl group as the second terminal group can be referred to as H/C₁₈PEOXABP60-OH.

A polymer, H/C₁₈PEOXABP60, having an H/C₁₈ of about 0.7 was terminated with EDA with a molar ratio of polyoxazoline reactive chain end to EDA of about 1:10, producing a polymer having a second terminal group comprising a group modified with a primary amine (herein referred to as “Polymer B1”). Aqueous solutions of Polymer B1 had pH values in a range of from 7.0 to 10. Another aqueous solution of Polymer B1 can be adjusted to have pH values in a range of from 8.9 to 9.7 using HCl or NaOH. Another aqueous solution of Polymer B1 can be adjusted to have pH values in a range of from 4.0 to 6.9.

A polymer, H/C₁₈PEOXABP60, having an H/C₁₈ of about 0.4 was terminated with EDA with a molar ratio of polyoxazoline reactive chain end to EDA of about 1:10, producing a polymer having the second terminal group comprising a group modified with a primary amine (herein referred to as “Polymer B2”). An aqueous solution of Polymer B2 can be adjusted to have pH values in a range of from 7.0 to 10. Another aqueous solution of Polymer B2 can be adjusted to have pH values in a range of from 4.0 to 10 or 5.6 to 7.5 using HCl or NaOH.

A polymer, H/C₁₈PEOXABP60, having an H/C₁₈ of about 0.3 was terminated with EDA with a molar ratio of polyoxazoline reactive chain end to EDA of about 1:10, producing a polymer having the second terminal group comprising a group modified with a primary amine (herein referred to as “Polymer B3”). An aqueous solution of Polymer B3 can be adjusted to have pH values in a range of from 7.0 to 10. Another aqueous solution of Polymer B3 can be adjusted to have pH values in a range of from 4.0 to 10 or 5.6 to 7.5 using HCl or NaOH.

All H/C₁₈PEOXABP60 polymers having an —NH₂ group as the second terminal group can be referred to as H/C₁₈PEOXABP60-NH₂.

### Nanoparticle Measurement

The size of various polymers, polymer-only nanoaggregates, as well as drug-induced polymer-drug nanoaggregates was measured by a dynamic light scattering (DLS) method using a Malvern Zetasizer Nano-ZS Zen3600 particle size analyzer (Malvern Panalytical Inc., Westborough, MA 01581, USA).

### Examples 1-2

Nanoparticle with H/C18PEOXABP60-A (Polymer A1) Polymer:Formula (1)

Adjuvant Formulation-1:Polymer A1 (200 mg) having an H/C18 of about 0.4 prepared according to the Polymers section above was dissolved in water to make a 100 mg/g solution. To the polymer solution was added the compound of Formula (1) (8 mg) and processed according to the process described above to produce an aqueous nanoaggregate solution containing 2 mg/ml Formula (1) having a weight ratio of polymer:Formula (1)=25:1. The solution was filtered through 0.8 and 0.2 μm filters and lyophilized over 20-100 h to provide a lyophilized dry power. The vial was stoppered and the ready-to-use white powder was stored at room temperature.

Adjuvant Formulation-2: Polymer A1 (400 mg) having an H/C18 of about 0.4 prepared as above was dissolved in water to make a 100 mg/g solution. To the polymer solution was added the compound of Formula (1) (8 mg) and processed according to the process described above to produce an aqueous nanoaggregate solution containing 2 mg/mL Formula (1) having a weight ratio of polymer:Formula (1)=50:1. The solution was filtered through 0.8 and 0.2 μm filters and lyophilized over 20-100 h to provide a lyophilized dry power. The vial was stoppered and the ready-to-use white powder was stored at room temperature.

### Examples 3-4

Nanoparticle with H/C18PEOXABP60-A (Polymer A2) Polymer:Formula (1)

Adjuvant Formulation-3:Polymer A2 (400 mg) having an H/C18 of about 0.4 prepared as above was dissolved in water to make a 100 mg/g solution. To the polymer solution was added the compound of Formula (1) (8 mg) and processed according to the process described above to produce an aqueous nanoaggregate solution containing 2 mg/mL Formula (1) having a weight ratio of polymer:Formula (1)=50:1. The solution was filtered through a 0.8/0.2 μm filter set and lyophilized over 20-100 h to provide a lyophilized dry power. The vial was stoppered and the ready-to-use white powder was stored at room temperature.

Adjuvant Formulation-4:Polymer A3 (400 mg) having an H/C18 of about 0.7 prepared as above was dissolved in water to make a 100 mg/g solution. To the polymer solution was added the compound of Formula (1) (8 mg) and processed according to the process described above to produce an aqueous nanoaggregate solution containing 2 mg/mL Formula (1) having a weight ratio of polymer:Formula (1)=50:1. The solution was filtered through 0.8 and 0.2 μm filters and lyophilized over 20-100 h to provide a lyophilized dry power. The vial was stoppered and the ready-to-use white powder was stored at room temperature.

### Example 5-6

Adjuvant Formulation-5:Polymer B3 (400 mg) having an H/C18 of about 0.3 prepared as above was dissolved in water to make a 100 mg/g solution. To the polymer solution was added the compound of Formula (1) (8 mg) and processed according to the process described above to produce an aqueous nanoaggregate solution containing 2 mg/mL Formula (1) having a weight ratio of polymer:Formula (1)=50:1. The solution was filtered through 0.8 and 0.2 μm filters and lyophilized over 20-100 h to provide a lyophilized dry power. The vial was stoppered and the ready-to-use white powder was stored at room temperature.

Adjuvant Formulation-6:Polymer B3 (200 mg) having an H/C18 of about 0.3 prepared as above was dissolved in water to make a 100 mg/g solution. To the polymer solution was added the compound of Formula (1) (8 mg) and processed according to the process described above to produce an aqueous nanoaggregate solution containing 2 mg/mL Formula (1) having a weight ratio of polymer:Formula (1)=25:1. The solution was filtered through 0.8 and 0.2 μm filters and lyophilized over 20-100 h to provide a lyophilized dry power. The vial was stoppered and the ready-to-use white powder was stored at room temperature.

The adjuvant formulations are collectively referred to as “Nano-Adjuvant” or “Nan-Ad”.

### Example 7

### Antigen Design, Construction, Expression and Purification

1. Construction of the RBD antigen fusion protein:

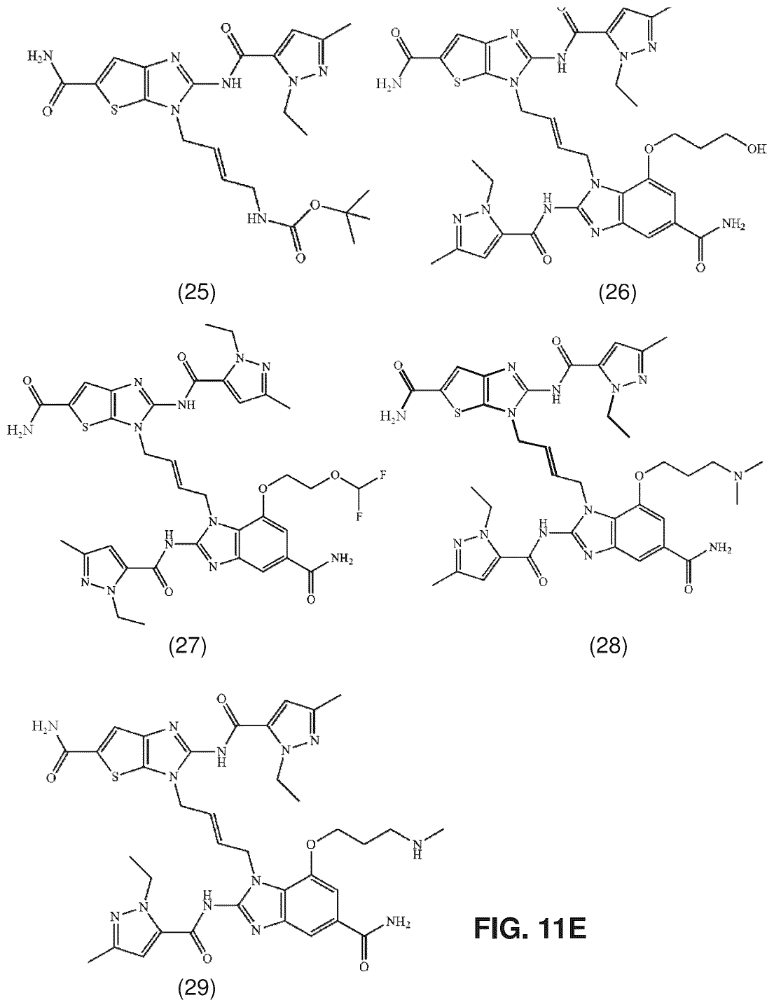

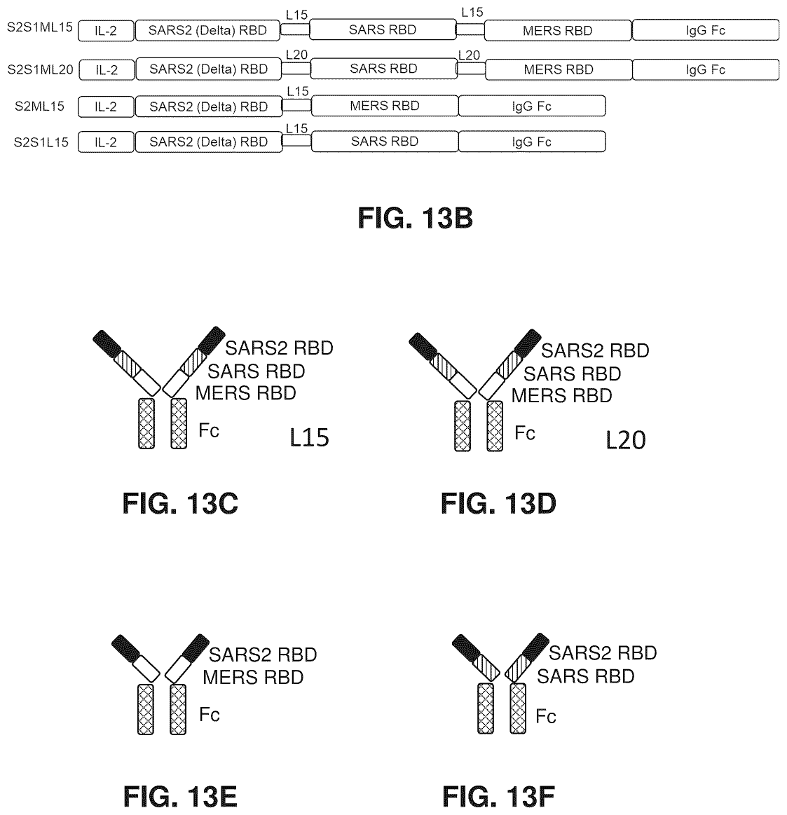

Two or more RBD (receptor-binding domain) antigen sequences from SARS-CoV-2 (Delta) (SEQ ID NO:1), SARS-CoV (SEQ ID NO:2), MERS-CoV (SEQ ID NO:3) were tandemly linked together via a linker sequence to form RBD antigen constructs. Examples of constructs with 3 RBD sequences, AG1 AG6, are schematically shown in FIG. 12. Linker 1 and Linker 2 can be the same or different. AG1 RBD antigen protein containing a linker, L20 (SEQ ID NO: 4: GGGGSGGGGSGGGGSGGGGS) with SARS-CoV-2 (Delta) RBD-linker-SARS-CoV RBD-linker-MERS-CoV RBD are listed in the Listing of Sequences below (SEQ ID NO:5). The nuclei acid construct for AG1 was cut with the restriction enzymes Eco RI and Nco1 and cloned into an expression vector, pFUSE-hlgG1-Fc2, fusing sequences of interest with the human IgG1Fc on the vector to form a fusion expression cassette (also referred to as “RBD antigen fusion protein expression cassette”). The fusion protein expression cassette (also referred to as “RBD antigen fusion protein expression cassette”) is named, “S2S1ML20 cassette” (S2: SARS-CoV-2 (Delta), S1: SARS-CoV, M: MERS-CoV and L20: linker L20). The expressed fusion protein (also referred to as “RBD antigen fusion protein”) from the S2S1ML20 cassette is referred to as S2S1ML20. Virus lineage is shown in FIG. 13A. Examples of expression cassettes and corresponding RBD antigen fusion proteins are schematically illustrated in FIG. 13B. Examples of antibodies having the fusion proteins are schematically illustrated in FIG. 13C-FIG. 13F: an example of an antigen fusion protein having 3 RBDs with L15 linkers (FIG. 13C), an example of an antigen fusion protein having 3 RBDs with L20 linkers (FIG. 13D), an example of an antigen fusion protein having 2 RBDs (SARS2 RBD and MERS RBD) (FIG. 13E) and another example of an antigen fusion protein having 2 RBDs (SARS2 RBD and SARS RBD) (FIG. 13F). The linkers can be positioned between the RBD sequences as shown in FIG. 13B. In the examples, the antigen fusion proteins were in a form of an immunoglobin resembling an IgG. A representative RBD antigen amino acid sequence alignment is shown in FIG. 14. As used herein, the term SARS or SARS-CoV refers to SARS-CoV and can be used interchangeably. The term SARS2 or SARS-CoV-2 refers to SARS-CoV-2. The term MERS or MERS-CoV refers to MERS-CoV.

Additional fusion proteins were also produced and included: S2S1ML 10 having SARS-CoV-2 (Delta) RBD-linker L10-SARS-CoV RBD-linker L10-MERS-CoV RBD (SEQ ID NO:6); S2S1ML15: SARS-CoV-2 (Delta) RBD-linker L15-SARS-CoV RBD-linker L15-MERS-CoV RBD (SEQ ID NO:7); S2ML15: SARS-CoV-2 (Delta) RBD-linker L15-MERS-CoV RBD (SEQ ID NO: 8); and S2SL15: SARS-CoV-2 (Delta) RBD-linker L15-SARS-CoV RBD (SEQ ID NO:9).

The linkers were: Linker L15 (SEQ ID NO: 10) and Linker L10 (SEQ ID NO: 11).

Additional RBD sequences including GenBank Accession number UJH58758.1, SARS-CoV-2 (WA1) RBD (SEQ ID NO:12); GenBank accession number UNE80990.1, SARS-CoV-2 (BA.2) RBD (SEQ ID NO:13); GenBank accession number MT040334.1, Pangolin coronavirus RBD (SEQ ID NO:14); GenBank accession number YP_009825051.1, SARS-CoV RBD (SEQ ID NO: 15); GenBank accession number AGZ48828.1, WIV1 RBD (SEQ ID NO: 16); GenBank accession number AGZ48818.1, Rs3367 RBD (SEQ ID NO: 17); GenBank accession number QJE50589.1, SHC014 RBD (SEQ ID NO: 18); GenBank accession number AFS88936.1, MERS-CoV Clade A (SEQ ID NO: 19); GenBank accession number AKJ80137.2, MERS-CoV Clade B (SEQ ID NO:20); and GenBank accession number AVN89387.1, MERS-CoV Clade C (SEQ ID NO:21), were also used as antigens or antigen fusion proteins.

2. Expression and purification of the RBD antigen fusion protein:

2.1. Grow the EXPi293F cells to a cell density of 1×10⁶/ml.

2.2. Transfect the cells with vector containing the S2S1ML20 cassette using transfection agent EsayTrans (available from Life-iLab).

2.3. Continue to culture the cells for 5 days. Then collect the supernatant by spinning down cells at 3000 rpm.

2.4. Purify the expressed S2S1ML20 using Protein A beads (available from GenScript) in a gravity column balanced with PBS (phosphate-buffered saline) with incubation at 4° C. for 2 hours. Wash with PBS. Elute protein with glycine solution.

2.5. Examine protein purity and size using SDS-PAGE.

### Example 8

### Nasal Vaccination

Fifteen BALB/c mice (female, 8 weeks of age, available from Vital River Laboratories) were randomly divided into 5 groups with 3 mice in each group. The mice were intranasally administrated with a vaccine having the antigen protein and an adjuvant formulation shown in Table 2. The mice were sacrificed according to approved protocols, 6 hours after application of the nasal vaccine components (also referred to as “nasal vaccination”). The lung specimens were homogenized with TRIzol and the total RNAs were extracted according to the manufacturer TRIzol method. RT-qPCT (Reverse transcription-quantitative polymerase chain reaction) kits (available from Takara) were used to quantify the mRNA levels of cytokines IFN-β, CXCL-10, CXCL-9, CCL-2, IL-6, IB-1β, IFN-g, TNF-α, CD40, CD86, TGFB-1, and IL-12 in the specimens.

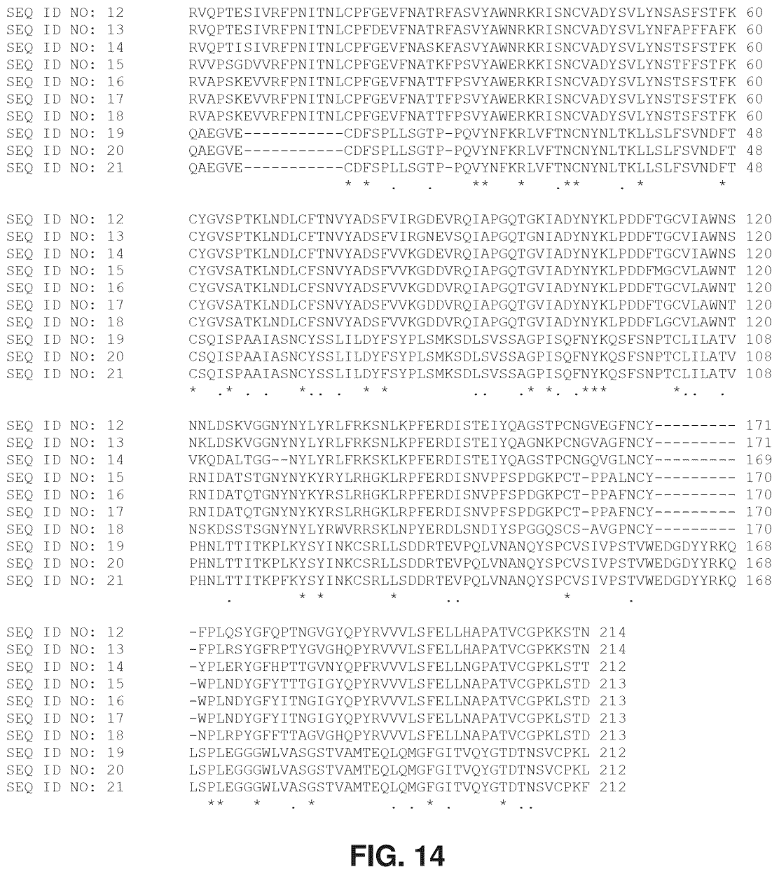

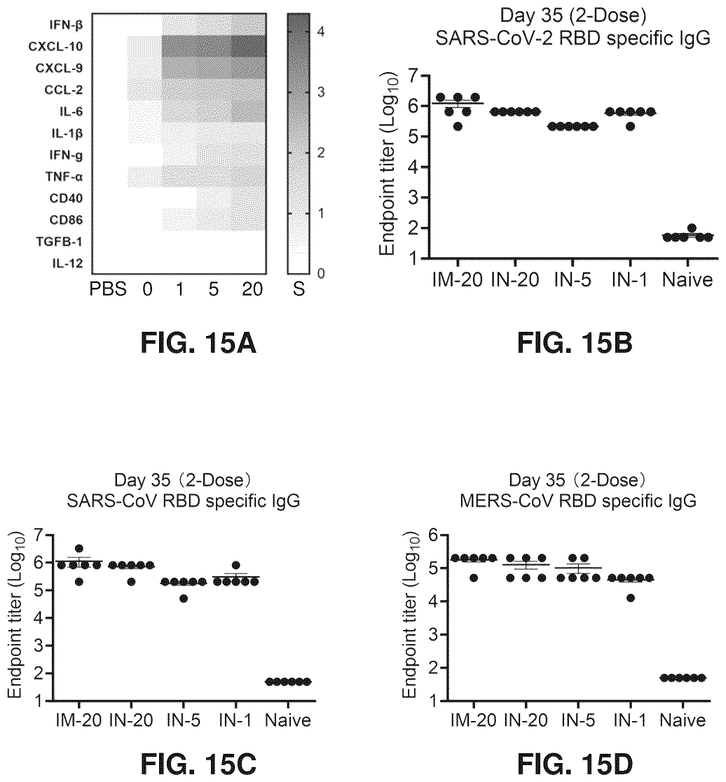

Representative results are shown in FIG. 15A with the Group ID indicated in Table 2 with the amount of the compound having the Formula (1) indicated. Immune response levels in induced cytokine production are reflected in the intensity shown in grey scales 1-4 (S: intensity scale) with darker grey representing higher level of the mRNAs.

The data indicated that the vaccine components having the RBD antigen protein and Adjuvant Formulation-3 were used for most of the tests. Other adjuvant Formulations prepared in Examples 1-6 above indicated similar stronger immune response when measured with cytokine production and other methods.

TABLE 2
Nasal vaccine components.

| | | Antigen Protein | |
|---|---|---|---|
| Group ID | PBS | S2S1ML20 | Nano-Adjuvant |
| PBS | 20 μL | — | — |
| 0 | 20 μL | 5 μg | — |
| 1 | 20 μL | 5 μg | 1 μg |
| 5 | 20 μL | 5 μg | 5 μg |
| 20 | 20 μL | 5 μg | 20 μg |

### Example 9

### Immune Response in Inducing β-Coronavirus Specific IgG

Thirty BALB/c mice (available from Vital River Laboratories) were randomly divided into 5 groups with 6 mice in each group. The mice were immunized with vaccine components shown in Table 3 in the administration route indicated. The naïve group received no vaccine.

Each of the mice was immunized at days 0, 28 and 56. Serum specimens were collected on days 35 and 63.

The collected serum specimens were heat-inactivated by heating to 56° C. for 30 min. Levels of IgG specific to the RBDs of SARS-CoV-2, SARS-CoV, and MERS-CoV were measured with ELISA. Briefly, the RBD proteins of SARS-CoV-2, SARS-CoV, and MERS-CoV were individually coated in the wells of ELISA plates, incubated overnight at 4° C. and then blocked with PBS (5% BSA, bovine serum albumin) for 2 hours at 37° C. The serum specimens were serially diluted and added to the wells of the blocked ELISA plates. The plates were incubated for 30 min at 37° C. and then washed 5 times with PBST (PBS with 0.05% Tween-20). An HRP-labelled rabbit anti-mouse IgG antibody was then added to the wells and incubated for 30 min at 37° C. The wells were washed 5 times with PBST, and then the color substrate, TMB, was added to the wells. After stopping the color reaction with H₂SO₄, the plates were measured for color intensity with an ELISA reader at A450.

Representative results are shown in FIG. 15B-FIG. 15D and Tables 4-6 from mice that received 2 doses of the vaccine components. Data indicated that both intramuscular (IM) injection and Intranasal (IN) inoculation induced strong immune responses as measured with IgG specific to the RBD proteins of SARS-CoV-2, SARS-CoV, and MERS-CoV. All P values were compared to the Naïve with One-way ANOVA. The amount of the adjuvant was based on the amount of the compound Formula (1) in the Adjuvant Formulation.

TABLE 3
Vaccine components.

| | | Antigen Protein | |
|---|---|---|---|
| Group ID | Administration | S2S1ML20 | Nano-Adjuvant |
| IM-20 | intramuscular (IM) | 5 μg | 20 μg |
| IN-20 | Intranasal (IN) | 5 μg | 20 μg |
| IN-5 | Intranasal (IN) | 5 μg | 5 μg |
| IN-1 | Intranasal (IN) | 5 μg | 1 μg |
| Naive | — | — | — |

TABLE 4
SARS-CoV-2 RBD specific IgG titer in mouse serum on day 35.

| Group ID | IM-20 | IN-20 | IN-5 | IN-1 | Naïve |
|---|---|---|---|---|---|
| RBD Specific | 1239300 ± | 656100 ± 0 | 218700 ± 0 | 583200 ± | 58.33 ± |
| titer ± SEM | 332475 | | | 72900 | 8.333 |
| P Value | P < 0.0001 | P < 0.0001 | P < 0.0001 | P < 0.0001 | / |

TABLE 5
SARS-CoV RBD specific IgG titer in mouse serum on day 35.

| Group ID | IM-20 | IN-20 | IN-5 | IN-1 | Naïve |
|---|---|---|---|---|---|
| RBD Specific | 1126400 ± | 716800 ± | 179200 ± | 307200 ± | 50 ± 0 |
| titer ± SEM | 441628 | 102400 | 25600 | 102400 | |
| P Value | P < 0.0001 | P < 0.0001 | P < 0.0001 | P < 0.0001 | / |

TABLE 6
MERS-CoV RBD specific IgG titer in mouse serum on day 35.

| Group ID | IM-20 | IN-20 | IN-5 | IN-1 | Naïve |
|---|---|---|---|---|---|
| RBD Specific | 179200 ± | 128000 ± | 102400 ± | 44800 ± | 50 ± 0 |
| titer ± SEM | 25600 | 34346 | 32382 | 6400 | |
| P Value | P < 0.0001 | P < 0.0001 | P < 0.0001 | P < 0.0001 | / |

Representative results are shown in FIG. 16A and Table 7 from the mice that received 3 doses of the vaccine components (63 days after the first administration of vaccine components). Data indicated that both intramuscular (IM) injection and Intranasal (IN) inoculation induced stronger immune responses as measured with the levels of SARS-CoV-2 RBD specific IgG. All P values were compared to the Naïve with One-way ANOVA.

TABLE 7
SARS-CoV-2 RBD specific IgG titer in mouse serum 63 days
after the first administration of vaccine components.

| Group ID | IM-20 | IN-20 | IN-5 | IN-1 | Naive |
|---|---|---|---|---|---|
| RBD Specific | 1433600 ± | 1638400 ± 0 | 409600 ± 0 | 409600 ± 0 | 50 ± 0 |
| titer ± SEM | 204800 | | | | |
| P Value | P < 0.0001 | P < 0.0001 | P < 0.0001 | P < 0.0001 | / |

### Example 10

### Immune Response in Inducing SARS-CoV-2 RBD Specific IgA

Levels of IgA specific to SARS-CoV-2 were measured with ELISA from the mice immunized above. Briefly, the RBD protein of SARS-CoV-2 was coated to the wells of ELISA plates by incubating overnight at 4° C. The same process was followed as described above except that an HRP-labelled rabbit anti-mouse IgA second antibody was used.

Representative results are provided in FIG. 16B and in Table 8. SARS-CoV-2 RBD specific IgA was not detected from mouse serum 63 days after intramuscular (IM) injection. However, Intranasal (IN) inoculation induced significant levels of SARS-CoV-2 RBD specific IgA in mouse serum.

TABLE 8
SARS-CoV-2 RBD specific IgA titer in mouse serum 63 days
after the first administration of vaccine components.

| Group ID | IM-20 | IN-20 | IN-5 | IN-1 | Naive |
|---|---|---|---|---|---|
| RBD Specific | 50 ± 0 | 1000 ± 268 | 800 ± 253 | 1600 ± 0 | 50 ± 0 |
| titer ± SEM | | | | | |
| P Value | P > 0.9999 | P < 0.0001 | P < 0.0001 | P < 0.0001 | / |

### Example 11

### Immune Response in Inducing SARS-CoV-2 RBD Specific IgA in the Lung

Levels of IgA specific to SARS-CoV-2 in the lung were measured with ELISA from the mice immunized above. Briefly, at day 63, 1 mL of PBS was introduced in the lung of the mice and bronchoalveolar lavage (BAL) fluid was collected. SARS-CoV-2 specific IgA was measured using ELISA as described above.

Representative results are shown in FIG. 16C and Table 9. Data indicated that SARS-CoV-2 specific IgA was present in the mouse BAL fluid after Intranasal (IN) inoculation.

TABLE 9
SARS-CoV-2 RBD specific IgA titer in mouse serum 63 days
after the first administration of vaccine components.

| Group ID | IN-20 | IN-5 | IN-1 | naïve |
|---|---|---|---|---|
| RBD Specific | 270 ± 0 | 210 ± 37 | 270 ± 0 | 5 ± 0 |
| titer ± SEM | | | | |
| P Value | P < 0.0001 | P < 0.0001 | P < 0.0001 | / |

### Example 12

Neutralization Antibody Titers in Mice Immunized with SARS-CoV-2 (D614G) with Different Adjuvants.

Mice were immunized with 5 μg of the RBD antigen fusion protein S2S1ML20 formulated with an adjuvant specified in Table 10. The Nano-Adjuvant (adjuvant formulation-3) comprising compound Formula (1) was prepared as described above. Neutralization antibody specific to SARS-CoV-2 (D614G) were assayed as described above. The data are shown in Table 10 and FIG. 16D. The data indicated that Nano-Adjuvant resulted in significantly higher, more than 10-fold, neutralization antibody titers when compared to traditional aluminum-containing adjuvant (available from Thermofisher). Intramuscular (IM) injection and intranasal (IN) immunization had similar results.

TABLE 10
Titers of neutralization antibodies from mice immunized
with RBD Antigen S2S1ML20 and different adjuvants.

| Group ID | IM-20 | IN-1 | Alum IM | Naive |
|---|---|---|---|---|
| Adjuvant | Nano- | Nano- | Alum | / |
| | Adjuvant | Adjuvant | Adjuvant | |
| | 20 μg | 1 μg | | |
| Immunization | Intramuscular | Intranasal | Intramuscular | / |
| Route | (IM) injection | (IN) | (IM) injection | |
| RBD Antigen | 5 μg | 5 μg | 5 μg | / |
| S2S1ML20 | | | | |
| Neutralization | 31525 ± | 19035 ± | 1670 ± | 50 ± 0 |
| Antibody | 9016 | 8079 | 214 | |
| Titer ± SD | | | | |
| P Value | P < 0.0001 | P < 0.0001 | P < 0.0001 | / |

### Example 13

Cellular Immune Response in the Lung after Intranasal (IN) Inoculation

Levels of IFN-γ and IL-4 that are specific to SARS-CoV-2, SARS-CoV, and MERS-CoV RBD were measured from the mice immunized with intranasal (IN) inoculation (nasal immunization) above shown as IN-1. Briefly, at day 63 after application of the nasal vaccine components, mice were sacrificed according to approved protocols. Lung specimens were collected and treated with Collagenase D and DNase I to produce a single lung cell suspension. The spleen specimens were collected and homogenized to produce a single spleen cell suspension. Red cells were removed with red cell lysis solution. Levels of IFN-γ and IL-4 that are specific to SARS-CoV-2, SARS-CoV, and MERS-CoV RBD were measured with the ELISPOT kit (available from MabTech).

Briefly, about 2×10⁵ lung or spleen cells were added to pre-coated wells of ELISPOT plates. About 2 μg of the SARS-CoV-2, SARS-CoV and MERS-CoV proteins were added to stimulate the cells and the plates incubated for 48 hours without shaking. The wells were then washed 5 times with PBS. Biotin-labelled IFN-γ and IL-4 antibodies were added and incubated for 2 hours. After washing with PBS, streptavidin labelled ALP (available from MabTech) was added and the plate incubated for 1 hour. Colorimetric phosphatase substrate was then added to the wells. Colored sites on the cells were counted.

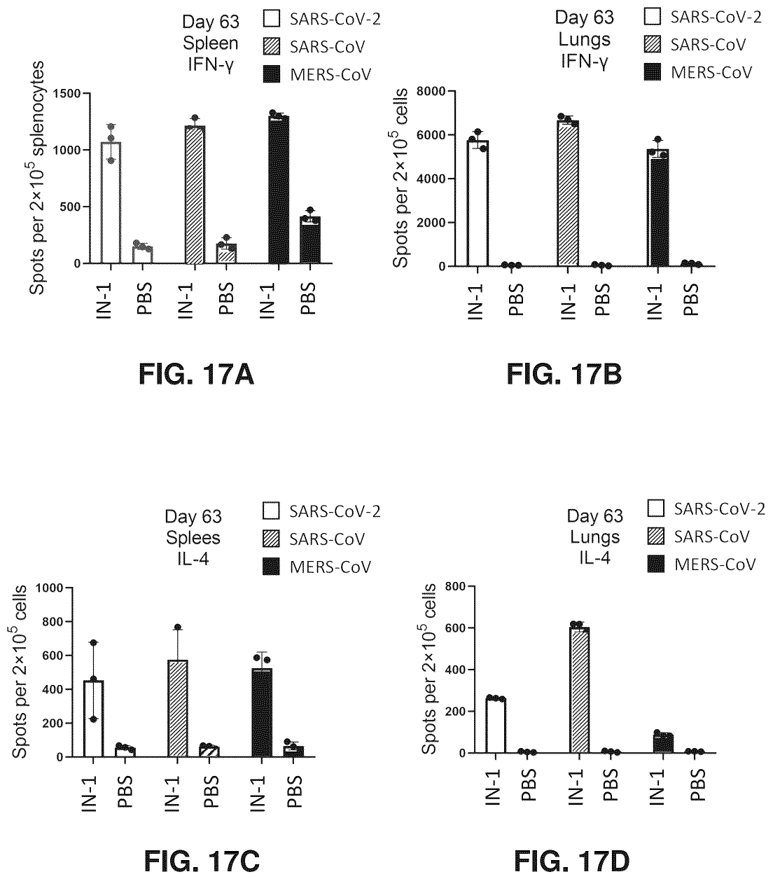

Representative results are shown in FIG. 17A-FIG. 17D. The data indicated that the vaccine components induced strong cellular immune responses in the lung as measured by SARS-CoV-2, SARS-CoV, and MERS-CoV RBD specific IFN-γ and IL-4 after intranasal (IN) inoculation. The data also indicted that SARS-CoV-2, SARS-CoV, and MERS-CoV specific immune responses were present in the spleen.

### Example 14

Dosage Level and IgG Immune Response with Intranasal (IN) and Intramuscular (IM) Immunization

Data shown above indicated that various dosages of the antigen and the Nan-Adjuvant induced broad immune responses in mice. The dosage of 2 μg of the Nano-Adjuvant (Adjuvant Formulation-3) was tested for intranasal (IN) immunization and 20 μg of the Nano-Adjuvant (Adjuvant Formulation-3) was tested for intramuscular (IM) immunization.

Briefly, 24 BALB/c mice were randomly divided into 4 groups with 6 mice in each group. The mice in Group 1-Group 3 (G1-G3) were administered with the vaccine dosages shown in Table 11 in the administration route indicated. The mice were immunized at day 0 and day 28. The Group 4 (G4) mice received no vaccine. On day 35, serum was collected from each of the mice. Levels of IgG specific to the RBD proteins of SARS-CoV-2, SARS-CoV, and MERS-CoV were measured as described above.

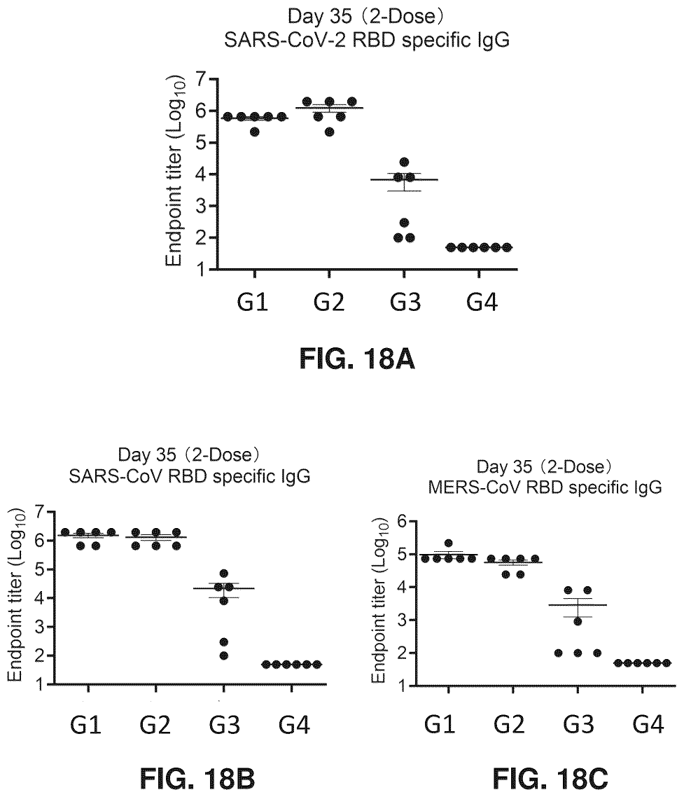

Representative results are shown in FIG. 18A-FIG. 18C and Tables 12-14. The data indicated that the Nano-Adjuvant at 2 μg for intranasal (IN) and 20 μg for intramuscular induced similar IgG levels with all viruses tested (G1 and G2). The vaccine having no adjuvant induced weaker response as shown with the lower IgG levels. All G1-G3 had significantly higher IgG levels as compared to the untreated group 4 (G4).

TABLE 11
Vaccine and immunization administration.

| | | Antigen Protein | |
|---|---|---|---|
| Group ID | Administration | S2S1ML20 | Nano-Adjuvant |
| G1 | Intramuscular (IM) | 5 μg | 20 μg |
| G2 | Intranasal (IN) | 5 μg | 2 μg |
| G3 | Intranasal (IN) | 5 μg | — |
| G4 | — | — | — |

TABLE 12
Titers of SARS-CoV-2 specific IgG 35
days after the first immunization.

| Group ID | G1 | G2 | G3 | G4 |
|---|---|---|---|---|
| RBD Specific | 583200 ± | 1239300 ± | 6833 ± | 50 ± 0 |
| titer ± SEM | 72900 | 332475 | 3837 | |
| P Value | P < 0.0001 | P < 0.0001 | P < 0.0001 | / |

TABLE 13
Titers of SARS-CoV specific IgG 35
days after the first immunization.

| Group ID | G1 | G2 | G3 | G4 |
|---|---|---|---|---|
| RBD Specific | 1530900 ± | 1312200 ± | 21667 ± | 50 ± 0 |
| titer ± SEM | 276636 | 293417 | 11172 | |
| P Value | P < 0.0001 | P < 0.0001 | P = 0.0012 | / |

TABLE 14
Titers of MERS-CoV specific IgG 35
days after the first immunization.

| Group ID | G1 | G2 | G3 | G4 |
|---|---|---|---|---|
| RBD Specific | 97200 ± | 56700 ± | 2900 ± | 50 ± 0 |
| titer ± SEM | 24300 | 10246 | 1649 | |
| P Value | P < 0.0001 | P < 0.0001 | P = 0.0028 | / |

### Example 15

Titer of Neutralizing Antibody with Intranasal (IN) Immunization

Neutralizing antibodies induced with the vaccines prepared herein were tested with a pseudovirus neutralization assay. In brief, pseudovirus containing the trimeric transmembrane spike (S) glycoprotein of coronavirus and luciferase were interacted with target cells (Huh-7 cells) that contain human angiotensin-converting enzyme 2 (ACE2), the SARS-CoV-2 receptor, in the presence of serum from the mice immunized with the vaccine described here. If the serum contains neutralizing antibody, luciferase activity will be reduced.

Mouse serum prepared from the Examples above was used.

1. Preparation of pseudovirus containing the trimeric transmembrane spike (S) glycoprotein of the coronavirus:

1.1. Grow HEK-293T to proper density for about 24 hours.

1.2. Transfect the HEK-293T cells with the Vigofect transfection reagent (available from Vigofect, China) and co-transfect with plasmid PNL-4-3 (available from NIH AIDS Reagent Program, US, catalog number: 3418) and a plasmid containing the S glycoprotein of an indicated virus (PcDNA3.1-SARS-CoV-2, PcDNA3.1-SARS-CoV or pcDNA3.1-MERS-CoV).

1.3. Change media after culturing for 12 hours.

1.4. Collect the culture supernatant containing the pseudovirus.

2. Neutralization assay with the pseudovirus:

2.1. Plate Huh-7 cells in 96-well plates at 10000 cells per well.

2.2. Serially dilute serum from mice with DMEM media.

2.3. Mix the pseudovirus and the serum, incubate at 37° C. for 30 min.

2.4. Add the pseudovirus-serum mix to the plate wells containing the HuH7 cells.

2.5. Measure light level generation with a Luciferase kit (available from Promega)

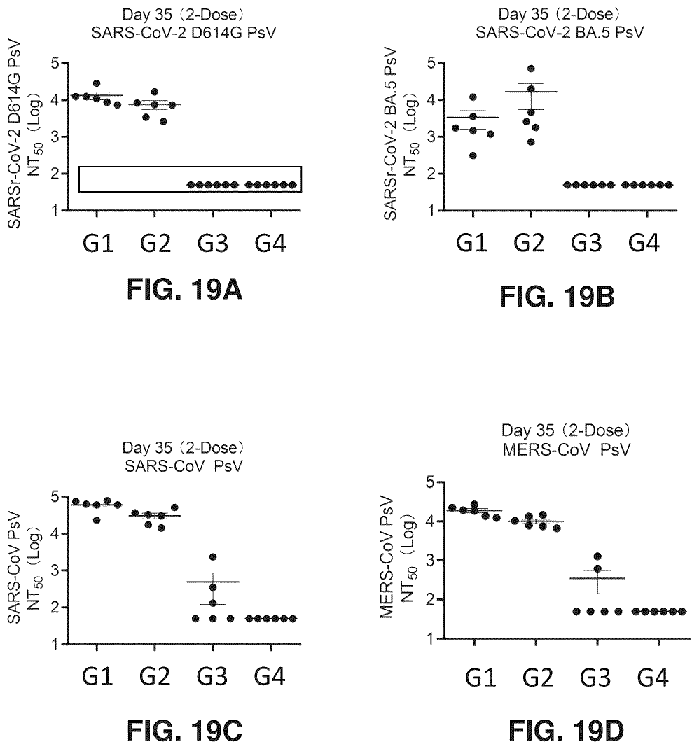

Representative results are shown in FIG. 19A-FIG. 19D and Tables 15-18. The data indicated the vaccine having the Nano-Adjuvant containing compound Formula (1) induced strong broad-spectrum neutralizing antibodies against the viruses, SARS-CoV-2 (strain D614G), SARS-CoV-2 Omicron (Strain BA.5), SARS-CoV, and MERS-CoV. No significant neutralizing antibodies were detected without the Nano-Adjuvant. The P values were compared to the levels with Group 4 (G4), mice not immunized.

TABLE 15
Titers of SARS-CoV-2 (strain D614G) neutralization
antibody 35 days after the first immunization.

| Group ID | G1 | G2 | G3 | G4 |
|---|---|---|---|---|
| Neutralizing | 13423 ± | 7673 ± | 50 ± 0 | 50 ± 0 |
| titer ± SEM | 3139 | 2056 | | |
| P Value | P < 0.0001 | P < 0.0001 | P > 0.9999 | / |

TABLE 16
Titers of SARS-CoV-2 (strain BA.5) neutralization
antibody 35 days after the first immunization.

| Group ID | G1 | G2 | G3 | G4 |
|---|---|---|---|---|
| Neutralizing | 3375 ± | 16654 ± | 50 ± 0 | 50 ± 0 |
| titer ± SEM | 1774 | 11125 | | |
| P Value | P < 0.0001 | P < 0.0001 | P > 0.9999 | / |

TABLE 17
Titers of SARS-CoV neutralization antibody
35 days after the first immunization.

| Group ID | G1 | G2 | G3 | G4 |
|---|---|---|---|---|
| Neutralizing | 59701 ± | 30293 ± | 493 ± 370 | 50 ± 0 |
| titer ± SEM | 8000 | 5495 | | |
| P Value | P < 0.0001 | P < 0.0001 | P = 0.07 | / |

TABLE 18
Titers of MERS-CoV neutralization antibody
35 days after the first immunization.

| Group ID | G1 | G2 | G3 | G4 |
|---|---|---|---|---|
| Neutralizing | 18804 ± | 10022 ± | 349 ± 207 | 50 ± 0 |
| titer ± SEM | 2251 | 1367 | | |
| P Value | P < 0.0001 | P < 0.0001 | P = 0.11 | / |

### Example 16

### IgG Immune Response in Monkey (Rhesus Macaques)

Three moneys (Macaca mulatta available from Beijing Vafory Technology Co., Ltd.) each was immunized with a vaccine comprising 100 μg of the RBD antigen fusion protein S2S1ML20 and Nano-Adjuvant (adjuvant formulation-3) comprising 400 μg of compound Formula (1) prepared as above. The monkeys were immunized at day 0 and day 28 via intramuscular (IM) injection. At days 14 and 35, specimens of serum, bronchoalveolar lavage (BAL) fluid (2 mL) and nasal washing fluid (NAL) (2 mL) were collected from each of the monkeys.

Monkey IgG specific to the virus SARS-CoV-2, SARS-CoV, or MERS-CoV RBD was measured using ELISA. Briefly:

1. Wells of ELISA plates were individually pre-coated with the SARS-CoV-2, SARS-CoV, or MERS-CoV RBD protein.

2. The wells were blocked with PBS 5% BSA for 2 hours at 37° C.

3. Specimens of serum, bronchoalveolar lavage (BAL) fluid and nasal washing fluid (NAL) were serially diluted and added to the wells. Incubate plates for 30 min at 37° C.

4. Wash the wells 5 times with PBST.

5. Add HRP-labelled rabbit-anti-monkey IgG antibody and incubate for 30 min at 37° C.

6. Wash the wells 5 times with PBST.

7. Add TMB substrate for color development.

8. Stop the reaction with H₂SO₄. Measure the color level with an ELISA reader at A450.

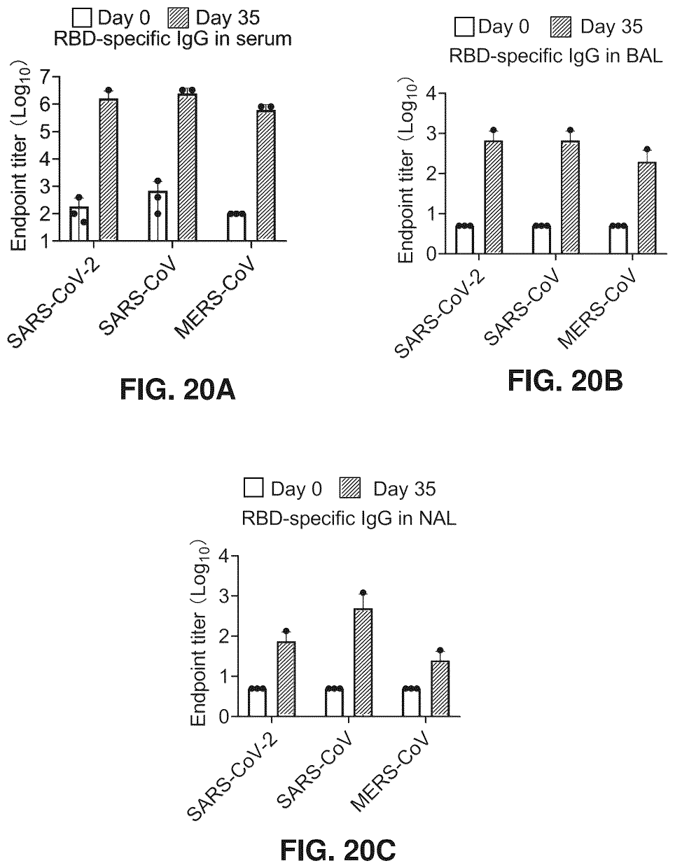

Representative results are shown in FIG. 20A-FIG. 20C and Tables 19-27. The data indicated the vaccine induced strong immune response as measured with the IgGs specific to SARS-CoV-2, SARS-CoV, and MERS-CoV RBD proteins. The data indicated the vaccine induced significant immune response in monkeys as measured with levels of IgG specific to the RBD proteins.

TABLE 19
Titers of SARS-CoV-2 RBD specific IgG in monkey
serum 35 days after the first immunization.

| Sample | Day 0 | Day 35 |
|---|---|---|
| RBD | 183 ± | 1638400 ± |
| Specific titer ± SEM | 109 | 819200 |
| P Value | / | P = 0.0003 |

TABLE 20
Titers of SARS-CoV RBD specific IgG in monkey
serum 35 days after the first immunization.

| Sample | Day 0 | Day 35 |
|---|---|---|
| RBD | 700 ± | 2457600 ± |
| Specific titer ± SEM | 458 | 819200 |
| P Value | / | P = 0.0008 |

TABLE 21
Titers of MERS-CoV RBD specific IgG in monkey
serum 35 days after the first immunization.

| Sample | Day 0 | Day 35 |
|---|---|---|
| RBD | 100 ± | 614400 ± |
| Specific titer ± SEM | 0 | 204800 |
| P Value | / | P < 0.0001 |

TABLE 22
Titers of SARS-CoV-2 RBD specific IgG in monkey
BAL 35 days after the first immunization.

| Sample | Day 0 | Day 35 |
|---|---|---|
| RBD | 5 ± 0 | 675 ± 270 |
| Specific titer ± SEM | | |
| P Value | / | P = 0.0002 |

TABLE 23
Titers of SARS-CoV RBD specific IgG in monkey
BAL 35 days after the first immunization.

| Sample | Day 0 | Day 35 |
|---|---|---|
| RBD | 5 ± 0 | 675 ± 270 |
| Specific titer ± SEM | | |
| P Value | / | P = 0.0002 |

TABLE 24
Titers of MERS-CoV RBD specific IgG in monkey
BAL 35 days after the first immunization.

| Sample | Day 0 | Day 35 |
|---|---|---|
| RBD | 5 ± 0 | 195 ± 108 |
| Specific titer ± SEM | | |
| P Value | / | P = 0.0065 |

TABLE 25
Titers of SARS-CoV-2 RBD specific IgG in monkey nasal washing
fluid (NAL) 35 days after the first immunization.

| Sample | Day 0 | Day 35 |
|---|---|---|
| RBD | 5 ± 0 | 75 ± 30 |
| Specific titer ± SEM | | |
| P Value | / | P = 0.0022 |

TABLE 26
Titers of SARS-CoV RBD specific IgG in monkey nasal washing
fluid (NAL) 35 days after the first immunization.

| Sample | Day 0 | Day 35 |
|---|---|---|
| RBD | 5 ± 0 | 495 ± 360 |
| Specific titer ± SEM | | |
| P Value | / | P = 0.0053 |

TABLE 27
Titers of MERS-CoV RBD specific IgG in monkey nasal washing
fluid (NAL) 35 days after the first immunization.

| Sample | Day 0 | Day 35 |
|---|---|---|
| RBD | 5 ± 0 | 25 ± 10 |
| Specific titer ± SEM | | |
| P Value | / | P = 0.0161 |

### Example 17

### Neutralization Antibody in Monkey (Rhesus Macaques)

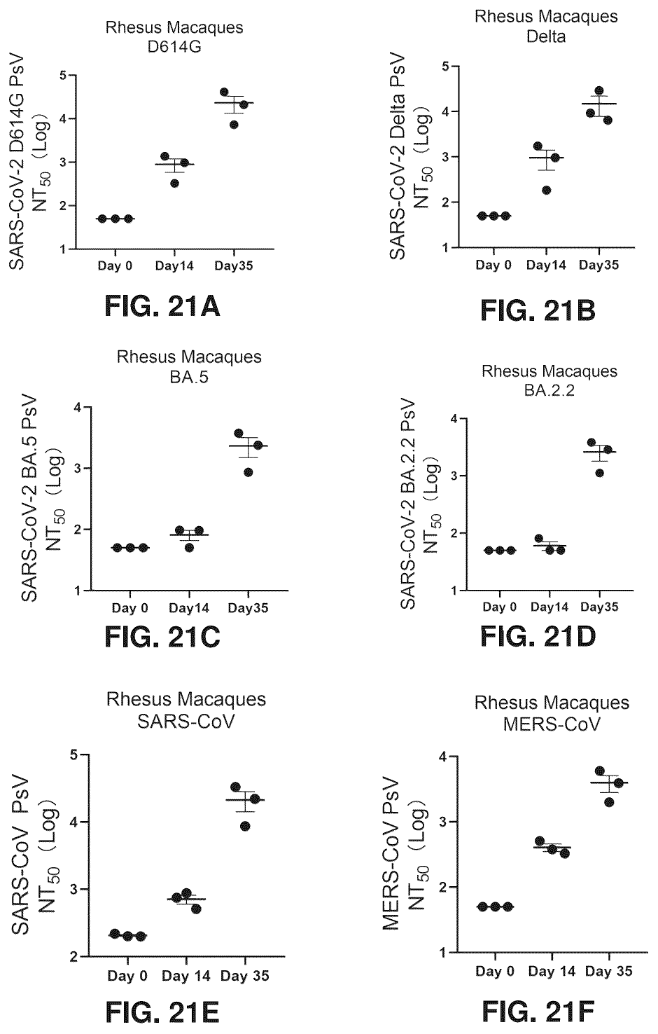

The neutralization antibody assay was performed as described above. Representative results are shown in FIG. 21A-FIG. 21F and Tables 28-33. The P values were compared to the specimens of Day 0. The data indicated that intramuscular (IM) injection immunization induced strong immune response measured with neutralizing antibodies against the viruses and variants tested including SARS-CoV, MERS-CoV and SARS-CoV-2 and variants.

TABLE 28
Titer of neutralization antibody specific to SARS-CoV-2 (Strain
D614G) in monkey serum 35 days after the first immunization.

| Sample | Day 0 | Day 14 | Day 35 |
|---|---|---|---|
| Neutralizing titer ± | 50 ± | 887.0 ± | 23141 ± |
| SEM | 0 | 303 | 9825 |
| P Value | / | 0.0044 | P < 0.0001 |

TABLE 29
Titer of neutralization antibody specific to SARS-CoV-2 (Strain
BA.5) in monkey serum 35 days after the first immunization.

| Sample | Day 0 | Day 14 | Day 35 |
|---|---|---|---|
| Neutralizing titer ± SEM | 50 ± | 81 ± | 2329 ± |
| | 0 | 15 | 833 |
| P Value | / | 0.375 | P < 0.0007 |

TABLE 30
Titer of neutralization antibody specific to SARS-CoV-2 (Strain
BA.2.2) in monkey serum 35 days after the first immunization.

| Sample | Day 0 | Day 14 | Day 35 |
|---|---|---|---|
| Neutralizing titer ± SEM | 50 ± | 60.33 ± | 2601 ± |
| | 0 | 10.33 | 793 |
| P Value | / | 0.8474 | P < 0.0001 |

TABLE 31
Titer of neutralization antibody specific to SARS-CoV-2 (Strain
Delta) in monkey serum 35 days after the first immunization.

| Sample | Day 0 | Day 14 | Day 35 |
|---|---|---|---|
| Neutralizing titer ± SEM | 50 ± | 959.8 ± | 14899 ± |
| | 0 | 448 | 7103 |
| P Value | / | 0.375 | P < 0.0001 |

TABLE 32
Titer of neutralization antibody specific to SARS-CoV
in monkey serum 35 days after the first immunization.

| Sample | Day 0 | Day 14 | Day 35 |
|---|---|---|---|
| Neutralizing titer ± | 205 ± | 709 ± | 21246 ± |
| SEM | 6 | 107 | 7070 |
| P Value | / | 0.0239 | P < 0.0001 |

TABLE 33
Titer of neutralization antibody specific to MERS-CoV
in monkey serum 35 days after the first immunization.

| Sample | Day 0 | Day 14 | Day 35 |
|---|---|---|---|
| Neutralizing titer ± | 50 ± | 405 ± | 3963 ± |
| SEM | 0 | 54 | 1157 |
| P Value | / | 0.0006 | P < 0.0001 |

LISTING OF SEQUENCES
SEQ ID NO: 1: SARS-CoV-2 (Delta)
GenBank: ON196573.1
SARS-CoV-2(Delta)RBD
RVQPTESIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYRY
RLFRKSNLKPFERDISTEIYQAGSKPCNGVEGFNCYFPLQSYGFQ
PTNGVGYQPYRVVVLSFELLHAPATVCGPKKSTN
SEQ ID NO: 2: SARS-CoV,
GenBank: YP 009825051.1
SARS-CoV RBD
NITNLCPFGEVFNATKFPSVYAWERKKISNCVADYSVLYNSTFFS
TFKCYGVSATKLNDLCFSNVYADSFVVKGDDVRQIAPGQTGVIAD
YNYKLPDDFMGCVLAWNTRNIDATSTGNYNYKYRYLRHGKLRPFE
RDISNVPFSPDGKPCTPPALNCYWPLNDYGFYTTTGIGYQPYRVV
VLSFELLNAPATVCGPKLSTDLIKNQCVNFNFNGLTGTGVLT
SEQ ID NO: 3: MERS-CoV
GenBank: AFS88936.1
MERS-CoV RBD
QAEGVECDFSPLLSGTPPQVYNFKRLVFTNCNYNLTKLLSLFSVN
DFTCSQISPAAIASNCYSSLILDYFSYPLSMKSDLSVSSAGPISQ
FNYKQSFSNPTCLILATVPHNLTTITKPLKYSYINKCSRLLSDDR
TEVPQLVNANQYSPCVSIVPSTVWEDGDYYRKQLSPLEGGGWLVA
SGSTVAMTEQLQMGFGITVQYGTDTNSVCPKL
SEQ ID NO: 4: Linker L20
Artificial sequence
GGGGSGGGGSGGGGSGGGGS
SEQ ID NO: 5: Full construct AG1 S2S1ML20.
SARS-CoV-2 (Delta) RBD-linker-SARS-CoV
RBD-linker-MERS-CoV RBD (FIG. 12)
(SEW ID NO: 5).
S2S1ML20
RVQPTESIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYRY
RLFRKSNLKPFERDISTEIYQAGSKPCNGVEGFNCYFPLQSYGFQ
PTNGVGYQPYRVVVLSFELLHAPATVCGPKKSTNGGGGSGGGGSG
GGGSGGGGSNITNLCPFGEVFNATKFPSVYAWERKKISNCVADYS
VLYNSTFFSTFKCYGVSATKLNDLCFSNVYADSFVVKGDDVRQIA
PGQTGVIADYNYKLPDDFMGCVLAWNTRNIDATSTGNYNYKYRYL
RHGKLRPFERDISNVPFSPDGKPCTPPALNCYWPLNDYGFYTTTG
IGYQPYRVVVLSFELLNAPATVCGPKLSTDLIKNQCVNFNFNGLT
GTGVLTGGGGSGGGGSGGGGSGGGGSQAEGVECDFSPLLSGTPPQ
VYNFKRLVFTNCNYNLTKLLSLFSVNDFTCSQISPAAIASNCYSS
LILDYFSYPLSMKSDLSVSSAGPISQFNYKQSFSNPTCLILATVP
HNLTTITKPLKYSYINKCSRLLSDDRTEVPQLVNANQYSPCVSIV
PSTVWEDGDYYRKQLSPLEGGGWLVASGSTVAMTEQLQMGFGITV
QYGTDTNSVCPKL
SEQ ID NO: 6: Full construct S2S1ML10.
S2S1ML10
RVQPTESIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYRY
RLFRKSNLKPFERDISTEIYQAGSKPCNGVEGFNCYFPLQSYGFQ
PTNGVGYQPYRVVVLSFELLHAPATVCGPKKSTNGGGGSGGGGSN
ITNLCPFGEVFNATKFPSVYAWERKKISNCVADYSVLYNSTFFST
FKCYGVSATKLNDLCFSNVYADSFVVKGDDVRQIAPGQTGVIADY
NYKLPDDFMGCVLAWNTRNIDATSTGNYNYKYRYLRHGKLRPFER
DISNVPFSPDGKPCTPPALNCYWPLNDYGFYTTTGIGYQPYRVVV
LSFELLNAPATVCGPKLSTDLIKNQCVNFNFNGLTGTGVLTGGGG
SGGGGSQAEGVECDFSPLLSGTPPQVYNFKRLVFTNCNYNLTKLL
SLFSVNDFTCSQISPAAIASNCYSSLILDYFSYPLSMKSDLSVSS
AGPISQFNYKQSFSNPTCLILATVPHNLTTITKPLKYSYINKCSR
LLSDDRTEVPQLVNANQYSPCVSIVPSTVWEDGDYYRKQLSPLEG
GGWLVASGSTVAMTEQLQMGFGITVQYGTDTNSVCPKL
SEQ ID NO: 7: Full construct S2S1ML15.
S2S1ML15
RVQPTESIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYRY
RLFRKSNLKPFERDISTEIYQAGSKPCNGVEGFNCYFPLQSYGFQ
PTNGVGYQPYRVVVLSFELLHAPATVCGPKKSTNGGGGSGGGGSG
GGGSNITNLCPFGEVFNATKFPSVYAWERKKISNCVADYSVLYNS
TFFSTFKCYGVSATKLNDLCFSNVYADSFVVKGDDVRQIAPGQTG
VIADYNYKLPDDFMGCVLAWNTRNIDATSTGNYNYKYRYLRHGKL
RPFERDISNVPFSPDGKPCTPPALNCYWPLNDYGFYTTTGIGYQP
YRVVVLSFELLNAPATVCGPKLSTDLIKNQCVNFNFNGLTGTGVL
TGGGGSGGGGSGGGGSQAEGVECDFSPLLSGTPPQVYNFKRLVFT
NCNYNLTKLLSLFSVNDFTCSQISPAAIASNCYSSLILDYFSYPL
SMKSDLSVSSAGPISQFNYKQSFSNPTCLILATVPHNLTTITKPL
KYSYINKCSRLLSDDRTEVPQLVNANQYSPCVSIVPSTVWEDGDY
YRKQLSPLEGGGWLVASGSTVAMTEQLQMGFGITVQYGTDTNSVC
PKL
SEQ ID NO: 8: Full construct S2ML15.
S2ML15
RVQPTESIVRFPNITNLCPFGEVENATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYRY
RLFRKSNLKPFERDISTEIYQAGSKPCNGVEGFNCYFPLQSYGFQ
PTNGVGYQPYRVVVLSFELLHAPATVCGPKKSTNGGGGSGGGGSG
GGGSQAEGVECDFSPLLSGTPPQVYNFKRLVFTNCNYNLTKLLSL
FSVNDFTCSQISPAAIASNCYSSLILDYFSYPLSMKSDLSVSSAG
PISQFNYKQSFSNPTCLILATVPHNLTTITKPLKYSYINKCSRLL
SDDRTEVPQLVNANQYSPCVSIVPSTVWEDGDYYRKQLSPLEGGG
WLVASGSTVAMTEQLQMGFGITVQYGTDTNSVCPKL
SEQ ID NO: 9: Full construct S2SL15.
S2S1L15
RVQPTESIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYRY
RLFRKSNLKPFERDISTEIYQAGSKPCNGVEGFNCYFPLQSYGFQ
PTNGVGYQPYRVVVLSFELLHAPATVCGPKKSTNGGGGSGGGGSG
GGGSNITNLCPFGEVFNATKFPSVYAWERKKISNCVADYSVLYNS
TFFSTFKCYGVSATKLNDLCFSNVYADSFVVKGDDVRQIAPGQTG
VIADYNYKLPDDFMGCVLAWNTRNIDATSTGNYNYKYRYLRHGKL
RPFERDISNVPFSPDGKPCTPPALNCYWPLNDYGFYTTTGIGYQP
YRVVVLSFELLNAPATVCGPKLSTDLIKNQCVNFNFNGLTGTGVL
T
SEQ ID NO: 10: Linker L15
Artificial sequence
L15: GGGGSGGGGSGGGGS
SEQ ID NO: 11: Linker L10
Artificial sequence
L10: GGGGSGGGGS
SEQ ID NO: 12
GenBank: UJH58758.1
SARS-CoV-2(WA1)RBD
RVQPTESIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYLY
RLFRKSNLKPFERDISTEIYQAGSTPCNGVEGFNCYFPLQSYGFQ
PTNGVGYQPYRVVVLSFELLHAPATVCGPKKSTN
SEQ ID NO: 13
GenBank: UNE80990.1
SARS-CoV-2(BA.2)RBD
RVQPTESIVRFPNITNLCPFDEVFNATRFASVYAWNRKRISNCVA
DYSVLYNFAPFFAFKCYGVSPTKLNDLCFTNVYADSFVIRGNEVS
QIAPGQTGNIADYNYKLPDDFTGCVIAWNSNKLDSKVGGNYNYLY
RLFRKSNLKPFERDISTEIYQAGNKPCNGVAGFNCYFPLRSYGFR
PTYGVGHQPYRVVVLSFELLHAPATVCGPKKSTN
SEQ ID NO: 14
GenBank: MT040334.1
Pangolin coronavirus RBD
RVQPTISIVRFPNITNLCPFGEVENASKFASVYAWNRKRISNCVA
DYSVLYNSTSFSTFKCYGVSPTKLNDLCFTNVYADSFVVKGDEVR
QIAPGQTGVIADYNYKLPDDFTGCVIAWNSVKQDALTGGNYLYRL
FRKSKLKPFERDISTEIYQAGSTPCNGQVGLNCYYPLERYGFHPT
TGVNYQPFRVVVLSFELLNGPATVCGPKLSTT
SEQ ID NO: 15
GenBank: YP 009825051.1
SARS-CoV RBD
RVVPSGDVVRFPNITNLCPFGEVFNATKFPSVYAWERKKISNCVA
DYSVLYNSTFFSTFKCYGVSATKLNDLCFSNVYADSFVVKGDDVR
QIAPGQTGVIADYNYKLPDDFMGCVLAWNTRNIDATSTGNYNYKY
RYLRHGKLRPFERDISNVPFSPDGKPCTPPALNCYWPLNDYGFYT
TTGIGYQPYRVVVLSFELLNAPATVCGPKLSTD
SEQ ID NO: 16
GenBank: AGZ48828.1
WIV1 RBD
RVAPSKEVVRFPNITNLCPFGEVFNATTFPSVYAWERKRISNCVA
DYSVLYNSTSFSTFKCYGVSATKLNDLCFSNVYADSFVVKGDDVR
QIAPGQTGVIADYNYKLPDDFTGCVLAWNTRNIDATQTGNYNYKY
RSLRHGKLRPFERDISNVPFSPDGKPCTPPAFNCYWPLNDYGFYI
TNGIGYQPYRVVVLSFELLNAPATVCGPKLSTD
SEQ ID NO: 17
GenBank: AGZ48818.1
Rs3367 RBD
RVAPSKEVVRFPNITNLCPFGEVENATTFPSVYAWERKRISNCVA
DYSVLYNSTSFSTFKCYGVSATKLNDLCFSNVYADSFVVKGDDVR
QIAPGQTGVIADYNYKLPDDFTGCVLAWNTRNIDATQTGNYNYKY
RSLRHGKLRPFERDISNVPFSPDGKPCTPPAFNCYWPLNDYGFYI
TNGIGYQPYRVVVLSFELLNAPATVCGPKLSTD
SEQ ID NO: 18
GenBank: QJE50589.1
SHC014 RBD
RVAPSKEVVRFPNITNLCPFGEVENATTFPSVYAWERKRISNCVA
DYSVLYNSTSFSTFKCYGVSATKLNDLCFSNVYADSFVVKGDDVR
QIAPGQTGVIADYNYKLPDDFLGCVLAWNTNSKDSSTSGNYNYLY
RWVRRSKLNPYERDLSNDIYSPGGQSCSAVGPNCYNPLRPYGFFT
TAGVGHQPYRVVVLSFELLNAPATVCGPKLSTD
SEQ ID NO: 19
GenBank: AFS88936.1
MERS-CoV Clade A
QAEGVECDFSPLLSGTPPQVYNFKRLVFTNCNYNLTKLLSLFSVN
DFTCSQISPAAIASNCYSSLILDYFSYPLSMKSDLSVSSAGPISQ
FNYKQSFSNPTCLILATVPHNLTTITKPLKYSYINKCSRLLSDDR
TEVPQLVNANQYSPCVSIVPSTVWEDGDYYRKQLSPLEGGGWLVA
SGSTVAMTEQLQMGFGITVQYGTDTNSVCPKL
SEQ ID NO: 20
GenBank: AKJ80137.2
MERS-CoV Clade B
QAEGVECDFSPLLSGTPPQVYNFKRLVFTNCNYNLTKLLSLFSVN
DFTCSQISPAAIASNCYSSLILDYFSYPLSMKSDLSVSSAGPISQ
FNYKQSFSNPTCLILATVPHNLTTITKPLKYSYINKCSRLLSDDR
TEVPQLVNANQYSPCVSIVPSTVWEDGDYYRKQLSPLEGGGWLVA
SGSTVAMTEQLQMGFGITVQYGTDTNSVCPKL
SEQ ID NO: 21
GenBank: AVN89387.1
MERS-CoV Clade C
QAEGVECDFSPLLSGTPPQVYNFKRLVFTNCNYNLTKLLSLFSVN
DFTCSQISPAAIASNCYSSLILDYFSYPLSMKSDLSVSSAGPISQ
FNYKQSFSNPTCLILATVPHNLTTITKPFKYSYINKCSRLLSDDR
TEVPQLVNANQYSPCVSIVPSTVWEDGDYYRKQLSPLEGGGWLVA
SGSTVAMTEQLQMGFGITVQYGTDTNSVCPKF

What is claimed is:

1. A pharmaceutical composition comprising:
a nanoaggregate comprising a polymer and at least one bioactive agent comprising at least one STING polypeptide or a part thereof, a nucleic acid encoding said STING polypeptide or a part thereof, a STING inhibitor, a STING activator, a STING agonist, a STING antagonist, a STING modulating molecule, or a combination thereof; and
optionally a pharmaceutical suitable carrier;
wherein said pharmaceutical composition is soluble in an aqueous solution to produce at least 1 mg/ml of said bioactive agent in said aqueous solution;
wherein said polymer is water soluble; and
wherein said polymer comprises:
a first polymer comprising at least one first terminal group modified with H or a hydrophobic moiety and a second terminal group modified with a hydrophilic moiety, wherein said first terminal group comprises in a range of from 1% to 100% of H and 0% to 99% of said hydrophobic moiety that comprises saturated or unsaturated aliphatic hydrocarbon having 1 to about 22 carbons, an aromatic hydrocarbon, or a combination thereof, and said second terminal group comprises a group modified by an amine, amide, imine, imide, carboxyl, hydroxyl, ester, ether, acetate, phosphate, ketone, aldehyde, sulfonate, or a combination thereof;
a second polymer; or
a combination thereof.

2. The pharmaceutical composition of claim 1, wherein said polymer comprises said first polymer.

3. The pharmaceutical composition of claim 1, wherein said second polymer comprises one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); poly(propylene oxide)-poly(ethylene oxide) (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or a combination thereof.

4. The pharmaceutical composition of claim 1, wherein said polymer comprises a polyoxazoline (POX) that comprises a linear portion, a branched portion, or a combination thereof, and wherein said polyoxazoline (POX) comprises poly(2-methyloxazoline), poly(2-ethyloxazoline), poly(2-propyloxazoline), poly(isopropyloxazoline), or a combination thereof.

5. The pharmaceutical composition of claim 4, wherein said polyoxazoline is poly(2-ethyloxazoline).

6. The pharmaceutical composition of claim 4, wherein said polyoxazoline comprises a molar ratio of monomer to initiator in a range of from 50:1 to 80:1.

7. The pharmaceutical composition of claim 1, wherein from 1% to 100% of said second terminal group is free from primary amine.

8. The pharmaceutical composition of claim 1, wherein from 1% to 100% of said second terminal group comprises hydroxyl group.

9. The pharmaceutical composition of claim 1, wherein said nanoaggregate is of a size less than 120 nm before lyophilization.

10. The pharmaceutical composition of claim 1, wherein said nanoaggregate has a weight ratio of said polymer to said bioactive agent in a range of from about 2:1 to about 200:1.

11. The pharmaceutical composition of claim 1, wherein said nanoaggregate is free from human serum albumin, organic solvent, detergent, or oil.

12. The pharmaceutical composition of claim 1, wherein said pharmaceutical composition is free from human serum albumin, organic solvent, detergent, or oil.

13. The pharmaceutical composition claim 1, wherein said pharmaceutical composition is a drug for treating or preventing a disease selected from immune disorders, infectious diseases, and a combination thereof.

14. The pharmaceutical composition of claim 1, wherein said bioactive agent comprises at least a compound having Formula (1)-Formula (29), a pharmaceutically acceptable salt thereof, solvate thereof, prodrug thereof, isomer thereof, or a combination thereof.

15. The pharmaceutical composition of claim 1, wherein said bioactive agent comprises a compound having Formula (1) 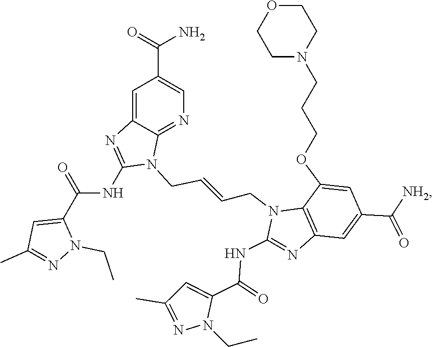 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21]
or Formula (4) 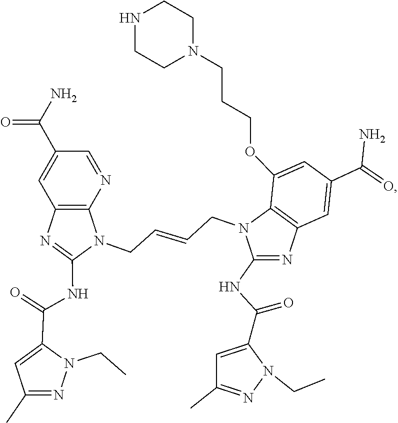 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]
or a pharmaceutically acceptable salt thereof, solvate thereof, prodrug thereof, isomer thereof, or a combination thereof.

16. The pharmaceutical composition of claim 1, wherein said pharmaceutical composition is an adjuvant for a vaccine.

17. The pharmaceutical composition of claim 16, wherein said pharmaceutical composition is a prophylactic vaccine, a therapeutic vaccine, or a combination thereof, wherein said pharmaceutical composition comprises said adjuvant and further comprises at least one immune agent for stimulating immune response in a subject in need thereof.

18. The pharmaceutical composition of claim 17, wherein said immune agent comprises inactive microbe selected from bacteria, viruses, fungi, protozoa, worms, parasites, prions, a part thereof, or a combination thereof; toxins; nucleic acids encoding said toxins; proteins; nucleic acids encoding said proteins; oligo nucleic acids; DNAs; RNAs; mRNAs; siRNAs; sgRNAs; fragments thereof; or a combination thereof.

19. The pharmaceutical composition of claim 15, wherein said pharmaceutical composition is formulated for treating or preventing at least one infectious disease.

20. The pharmaceutical composition of claim 19, wherein said pharmaceutical composition is formulated for treating or preventing at least one infectious disease selected from Chickenpox (Varicella), Coronaviruses, Dengue, Diphtheria, Ebola, Flu (Influenza), Hepatitis, Hib Disease, HIV/AIDS, HPV (Human Papillomavirus), Japanese Encephalitis, Measles, Meningococcal Disease, Monkeypox, Mumps, Norovirus, Pneumococcal Disease, Polio, Rabies, Respiratory Syncytial Virus (RSV), Rotavirus, Rubella (German Measles), Shingles (Herpes zoster), Tetanus (Lockjaw), Whooping Cough (Pertussis), Zika, and a combination thereof.

21. The pharmaceutical composition of claim 20, wherein said immune agent comprises at least a polypeptide of spike (S) glycoprotein of a coronavirus, a DNA encoding said spike (S) glycoprotein, an RNA encoding said spike (S) glycoprotein, a receptor-binding domain (RBD) of said spike (S) glycoprotein, a DNA encoding said RBD, an RNA encoding said RBD, a part thereof, or a combination thereof.

22. The pharmaceutical composition of claim 21, wherein said coronavirus comprises 229E α-coronavirus, NL63 α-coronavirus, OC43 β-coronavirus, HKU1 β-coronavirus, MERS-CoV, SARS-CoV, SARS-CoV-2, a variant thereof, or a combination thereof.

23. The pharmaceutical composition of claim 21, wherein said immune agent comprises at least one of said receptor-binding domain (RBD) of said spike (S) glycoprotein of said 229E α-coronavirus, NL63 α-coronavirus, OC43 β-coronavirus, HKU1 β-coronavirus, MERS-CoV, SARS-CoV, SARS-CoV-2, a variant thereof, or a combination thereof.

24. The pharmaceutical composition of claim 23, wherein said immune agent comprises at least a polypeptide of SEQ ID NO:1, SEQ ID NO:2, SEQ ID NO: 3, SEQ ID NO:5, SEQ ID NO:6, SEQ ID NO:7, SEQ ID NO:8, SEQ ID NO: 9, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO:20, SEQ ID NO:21, or a combination thereof.

25. The pharmaceutical composition of claim 24, wherein said immune agent comprises at least a polypeptide of said SEQ ID NO:5, SEQ ID NO:6, SEQ ID NO:7, SEQ ID NO:8, or SEQ ID NO:9.

26. The pharmaceutical composition of claim 17, wherein a weight ratio of said immune agent:said adjuvant is in a range of from 1:50 to 50:1, wherein said weight ratio is based on the weight of said immune agent and said bioactive agent acting as said adjuvant.

27. The pharmaceutical composition of claim 1, wherein said pharmaceutical composition further comprises one or more subsequent bioactive agents selected from a protein, a peptide, an antibody, a fragment of an antibody, a chemical compound, a small molecule drug, one or more chemotherapy drugs, and a combination thereof.

28. A method for treating or preventing a disease of a subject in need thereof, said method comprising administering said subject with an effective dose of a pharmaceutical composition comprising:
a nanoaggregate comprising a polymer and at least one bioactive agent comprising at least one STING polypeptide or a part thereof, a nucleic acid encoding said STING polypeptide or a part thereof, a STING inhibitor, a STING activator, a STING agonist, a STING antagonist, a STING modulating molecule, or a combination thereof; and
optionally a pharmaceutical suitable carrier;
wherein said pharmaceutical composition is soluble in an aqueous solution to produce at least 1 mg/ml of said bioactive agent in said aqueous solution;
wherein said polymer is water soluble; and
wherein said polymer comprises:
a first polymer comprising at least one first terminal group modified with H or a hydrophobic moiety and a second terminal group modified with a hydrophilic moiety, wherein said first terminal group comprises in a range of from 1% to 100% of H and 0% to 99% of said hydrophobic moiety that comprises saturated or unsaturated aliphatic hydrocarbon having 1 to about 22 carbons, an aromatic hydrocarbon, or a combination thereof, and said second terminal group comprises a group modified by an amine, amide, imine, imide, carboxyl, hydroxyl, ester, ether, acetate, phosphate, ketone, aldehyde, sulfonate, or a combination thereof; or
a second polymer comprising one or more hydroxyl dendrimers (HD); ethylene diamine-core poly(amidoamine) (PAMAM) hydroxyl-terminated generation-4, 5, 6, 7, 8, 9, 10 dendrimers, or a combination thereof; poly(ethylene glycol) (PEG); poly(lactic acid) (PLA); poly(lactic-co-glycolic acid) (PLGA); poly(propylene oxide) (PPO); poly(caprolactone) (PCL); poly(propylene oxide)-poly(ethylene oxide) (PPO-PEO); poly(γ-L-glutamic acid) (PGA); poly(L-phenylalanine ethyl ester) (PAE); poly(L-Lysine) (PLL); methyl-PEG (mPEG); poly(aspartamic acid) (PasP); poly(L-histidine) (PLH); poly(ethylene amine) (PEI); poly(N-vinylpyrrolidone) (PVP); poly(L-Leucine) (PLLeu); deoxycholic acid (DOCA); hydroxy propyl methyl cellulose (HPMC); poly(hydroxy butyrate) (PHB); poly(ethylene oxide) (PEO); poly(γ-benzyl-L-glutamate) (PBLG); phosphatidylserine (PS); poly(isohexyl-cyanoacrylate) (PIHCA); poly(allylamine hydrochlorine) (PAH); poly(γ-propargyl) (PP); or
a combination thereof.
